SEQUENCE LISTING (99 sequences)

SEQ ID NO: 1 - protein, 122 aa, synthetic construct
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSS

SEQ ID NO: 2 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 3 - protein, 107 aa, synthetic construct
AIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIK

SEQ ID NO: 4 - protein, 107 aa, synthetic construct [sequence omitted]

SEQ ID NO: 5 - protein, 814 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 7 - protein, 450 aa, synthetic construct
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 8 - protein, 214 aa, synthetic construct
AIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 936 aa, synthetic construct
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 450 aa, synthetic construct
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 11 - protein, 930 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - protein, 444 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 13 - protein, 214 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 444 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 15 - protein, 122 aa, synthetic construct; identical to SEQ ID NO: 1

SEQ ID NO: 16 - protein, 328 aa, synthetic construct; identical to SEQ ID NO: 2

SEQ ID NO: 17 - protein, 107 aa, synthetic construct; identical to SEQ ID NO: 3

SEQ ID NO: 18 - protein, 107 aa, synthetic construct [sequence omitted]

SEQ ID NO: 19 - protein, 814 aa, synthetic construct; identical to SEQ ID NO: 5

SEQ ID NO: 20 - protein, 328 aa, synthetic construct; identical to SEQ ID NO: 6

SEQ ID NO: 21 - protein, 450 aa, synthetic construct; identical to SEQ ID NO: 7

SEQ ID NO: 22 - protein, 214 aa, synthetic construct; identical to SEQ ID NO: 8

SEQ ID NO: 23 - protein, 936 aa, synthetic construct; identical to SEQ ID NO: 9

SEQ ID NO: 24 - protein, 450 aa, synthetic construct; identical to SEQ ID NO: 10

SEQ ID NO: 25 - protein, 930 aa, synthetic construct; identical to SEQ ID NO: 11

SEQ ID NO: 26 - protein, 444 aa, synthetic construct; identical to SEQ ID NO: 12

SEQ ID NO: 27 - protein, 214 aa, synthetic construct; identical to SEQ ID NO: 13

SEQ ID NO: 28 - protein, 444 aa, synthetic construct; identical to SEQ ID NO: 14

SEQ ID NO: 29 - protein, 118 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 30 - protein, 107 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIK

SEQ ID NO: 31 - protein, 116 aa, synthetic construct
EVQLVESGGGLVLPGRSMKLSCAASGFTFRTYYMAWVRQAPTKGLEWVASISIGGDNTYYRDSVMGRFTISRDDAKSTLHLQMDNLRSEDTATYYCVRLRGYFDYWGQGVMVTVSS

SEQ ID NO: 32 - protein, 106 aa, synthetic construct
EIVLTQSPATMPASPGEKVTLTCRASSSIRYIYWYQQKSGTSPKLWIYDTSKLASGVPNRFSGSGSGTSYSLTISSMETEDTATYYCQQGRSYPLTFGSGTKLEIK

SEQ ID NO: 33 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 34 - protein, 127 aa, synthetic construct
RVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSS

SEQ ID NO: 35 - protein, 107 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIK

SEQ ID NO: 36 - protein, 118 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKISKTQGFDYWGQGTLVTVSS

SEQ ID NO: 37 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQSRTRRPTFGQGTKVEIK

SEQ ID NO: 38 - protein, 119 aa, synthetic construct
EVQLVESGGGLVQPGGALRLSCSASGFTFSGYWMHWVRQAPGKGLEWVSQISSSGTIIDSADFVKGRFAVSRDNAKDLLYLQMNSLRADDTAVYYCARGPLAKNGFDIWGQGTMVTVSS

SEQ ID NO: 39 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIK

SEQ ID NO: 40 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 41 - protein, 446 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 42 - protein, 444 aa, synthetic construct
EVQLVESGGGLVLPGRSMKLSCAASGFTFRTYYMAWVRQAPTKGLEWVASISIGGDNTYYRDSVMGRFTISRDDAKSTLHLQMDNLRSEDTATYYCVRLRGYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 43 - protein, 450 aa, synthetic construct
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 44 - protein, 455 aa, synthetic construct
RVTLRESGPTLVKPTQTLTLTCTFSGFSLSTSGVGVGWIRQPPGKALEWLALIFWDDDSHYSPSLKSRLTITKDTSKNQVVLTMTNMDPVDTATYYCARYYFGSGSYFPSYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 45 - protein, 446 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKISKTQGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 46 - protein, 447 aa, synthetic construct
EVQLVESGGGLVQPGGALRLSCSASGFTFSGYWMHWVRQAPGKGLEWVSQISSSGTIIDSADFVKGRFAVSRDNAKDLLYLQMNSLRADDTAVYYCARGPLAKNGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 47 - protein, 444 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 48 - protein, 444 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 49 - protein, 214 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 213 aa, synthetic construct
EIVLTQSPATMPASPGEKVTLTCRASSSIRYIYWYQQKSGTSPKLWIYDTSKLASGVPNRFSGSGSGTSYSLTISSMETEDTATYYCQQGRSYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, synthetic construct; identical to SEQ ID NO: 8

SEQ ID NO: 52 - protein, 214 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 220 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQSRTRRPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 220 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 214 aa, synthetic construct; identical to SEQ ID NO: 13

SEQ ID NO: 56 - protein, 115 aa, synthetic construct
EVQLVQSGGGLVQPGGSLRLSCAASGFTFSNYWMTWVRQAPGEGPEWVANINQDGSQKYYVDSVKGRFTISRDNAKNSLNLQMNNLRAEDTALYYCAKSNAMDVWGQGTLVTVSS

SEQ ID NO: 57 - protein, 112 aa, synthetic construct
DIVMTQSPLSLPVTPGEPASISCRSSESLLHSNGYNYLNWYLQKPGQSPQLLIYLGSHRASGVPDRLSGSGSDTDFTLKISRVEAEDVGIYYCMQGRQTPITFGGGTKLEIK

SEQ ID NO: 58 - protein, 929 aa, synthetic construct
EVQLVQSGGGLVQPGGSLRLSCAASGFTFSNYWMTWVRQAPGEGPEWVANINQDGSQKYYVDSVKGRFTISRDNAKNSLNLQMNNLRAEDTALYYCAKSNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 443 aa, synthetic construct
EVQLVQSGGGLVQPGGSLRLSCAASGFTFSNYWMTWVRQAPGEGPEWVANINQDGSQKYYVDSVKGRFTISRDNAKNSLNLQMNNLRAEDTALYYCAKSNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 60 - protein, 219 aa, synthetic construct
DIVMTQSPLSLPVTPGEPASISCRSSESLLHSNGYNYLNWYLQKPGQSPQLLIYLGSHRASGVPDRLSGSGSDTDFTLKISRVEAEDVGIYYCMQGRQTPITFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 443 aa, synthetic construct
EVQLVQSGGGLVQPGGSLRLSCAASGFTFSNYWMTWVRQAPGEGPEWVANINQDGSQKYYVDSVKGRFTISRDNAKNSLNLQMNNLRAEDTALYYCAKSNAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 62 - protein, 134 aa, synthetic construct
QVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSSGGGGSGGGGS

SEQ ID NO: 63 - protein, 110 aa, synthetic construct
ARVDQTPQTITKETGESLTINCVLRDSNCALSSTYWYRKKSGSTNEENISKGGRYVETVNSGSKSFSLRINDLTVEDSGTYRCNVVQYPSYNNYFWCDVYGDGTAVTVNA

SEQ ID NO: 64 - protein, 233 aa, synthetic construct
GSTEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 65 - protein, 122 aa, synthetic construct; identical to SEQ ID NO: 1

SEQ ID NO: 66 - protein, 107 aa, synthetic construct; identical to SEQ ID NO: 3

SEQ ID NO: 67 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 68 - protein, 127 aa, synthetic construct
EVQLVESGGGLVQPGGSLRLSCAASGFTFDDYGMSWVRQAPGKALEWVSAISWNGNNTYYTESMKGRFTISRDNAKNTLYLQMNSLRPEDTAVYYCVKGSTAIVGVPPTYPDEYDYWGQGTLVTVSS

SEQ ID NO: 69 - protein, 360 aa, synthetic construct
EVQLVESGGGLVQPGGSLRLSCAASGFTFDDYGMSWVRQAPGKALEWVSAISWNGNNTYYTESMKGRFTISRDNAKNTLYLQMNSLRPEDTAVYYCVKGSTAIVGVPPTYPDEYDYWGQGTLVTVSSGSTEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 70 - protein, 367 aa, synthetic construct
QVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSSGGGGSGGGGSGSTEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 71 - protein, 343 aa, synthetic construct
ARVDQTPQTITKETGESLTINCVLRDSNCALSSTYWYRKKSGSTNEENISKGGRYVETVNSGSKSFSLRINDLTVEDSGTYRCNVVQYPSYNNYFWCDVYGDGTAVTVNAGSTEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQEGNVFSCSVMHEALHNHYTQKSLSLSP

SEQ ID NO: 72 - protein, 444 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 73 - protein, 450 aa, synthetic construct
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 74 - protein, 214 aa, synthetic construct; identical to SEQ ID NO: 13

SEQ ID NO: 75 - protein, 214 aa, synthetic construct; identical to SEQ ID NO: 8

SEQ ID NO: 76 - protein, 930 aa, synthetic construct; identical to SEQ ID NO: 11

SEQ ID NO: 77 - protein, 936 aa, synthetic construct; identical to SEQ ID NO: 9

SEQ ID NO: 78 - protein, 444 aa, synthetic construct; identical to SEQ ID NO: 14

SEQ ID NO: 79 - protein, 357 aa, synthetic construct
EVQLVESGGGLVQPGGSLRLSCAASGFTFDDYGMSWVRQAPGKALEWVSAISWNGNNTYYTESMKGRFTISRDNAKNTLYLQMNSLRPEDTAVYYCVKGSTAIVGVPPTYPDEYDYWGQGTLVTVSSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 80 - protein, 444 aa, synthetic construct; identical to SEQ ID NO: 12

SEQ ID NO: 81 - protein, 450 aa, synthetic construct; identical to SEQ ID NO: 10

SEQ ID NO: 82 - protein, 1132 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECMLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 83 - protein, 1132 aa, synthetic construct
AIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECMLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 84 - protein, 919 aa, synthetic construct
AIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 85 - protein, 448 aa, synthetic construct
EVQLVQSGAEVKKPGSSVKVSCKASGGTLSGYGVSWVRQAPGQGLEWMGNIIPGFGTANYAQKFQGRVTITADESTSTAYMELSSLRSEDTAVYYCARSGGTKYGMLDVWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDERVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 86 - protein, 923 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 87 - protein, 923 aa, synthetic construct
AIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 88 - protein, 215 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQLILWPPRTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 223 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 229 aa, synthetic construct
EVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 457 aa, synthetic construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 92 - protein, 943 aa, synthetic construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 93 - protein, 1192 aa, synthetic construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSGGGGSGGGGSGGGGSAIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIK

SEQ ID NO: 94 - protein, 457 aa, synthetic construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 95 - protein, 711 aa, synthetic construct
QVQLVQSGAEVKKPGASVKVSCKASGYTFTKYYMSWVRQAPGQGLEWMGIINPIGGSTSYAQKFQGRVTMTRDTSTSTVYMELSSLRSEDTAVYYCARDPSGIALAGPASRGYQGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSEVQLVESGGGLVQPGGSLRLSCAASGFTFSSYAMGWVRQAPGKGLEWVSAVSGSGGSTYYADSVKGRFTISRDNSKNTLYLQMNSLRAEDTAVYYCAKGGYDILTGYFFDYWGQGTTVTVSSGGGGSGGGGSGGGGSAIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIK

SEQ ID NO: 96 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQARLGPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 468 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQARLGPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGGLVQPGGSLRLSCAASGFTFSSYAMGWVRQAPGKCLEWVSAVSGSGGSTYYADSVKGRFTISRDNSKNTLYLQMNSLRAEDTAVYYCAKGGYDILTGYFFDYWGQGTTVTVSSGGGGSGGGGSGGGGSAIQMTQSPSSLSASVGDRVTITCRASQGISSALAWYQQKPGKAPKLLIYDASSLESGVPSRFSGSGSGTDFTLTISSLQPEDFATYYCQQFNSYPFTFGCGTRLEIK

SEQ ID NO: 98 - protein, 1102 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 99 - protein, 1102 aa, synthetic construct
AIQMTQSPSSLSASVGDRVTITCRASQGISSALAWYQQKPGKAPKLLIYDASSLESGVPSRFSGSGSGTDFTLTISSLQPEDFATYYCQQFNSYPFTFGPGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

(19) United States
(12) Patent Application Publication
(10) Pub. No.: US 2026/0062494 A1
(43) Pub. Date: Mar. 5, 2026

(54) ANTIGEN-BINDING MOLECULES WITH INCREASED PENETRATION INTO AND RETENTION IN BRAIN, AND METHODS FOR USE THEREOF

(71) Applicant: CHUGAI SEIYAKU KABUSHIKI KAISHA, Tokyo (JP)

(72) Inventors: Yuki NOGUCHI, Kanagawa (JP); Naoka HIRONIWA, Singapore (SG); Kazuki SATO, Kanagawa (JP)

(21) Appl. No.: 18/997,708

(22) PCT Filed: Aug. 3, 2023
(86) PCT No.: PCT/JP2023/028370

§ 371 Date: Jan. 22, 2025

(30) Foreign Application Priority Data
Aug. 4, 2022 (EP) 22188761.5
May 22, 2023 (EP) 23174571.2

(51) Int. Cl. C07K 16/28; A61K 39/00

(52) U.S. Cl. CPC C07K 16/2881; C07K 16/28; A61K 2039/505; C07K 2317/31; C07K 2317/94

(57) ABSTRACT

The present invention relates to antigen-binding molecules with increased penetration into and/or retention in brain; methods for use thereof; and methods for producing or screening thereof.

### TECHNICAL FIELD

The present invention relates to antigen-binding molecules with increased penetration into and/or retention in brain; methods for use thereof; and methods for producing or screening thereof. The present disclosure relates to antigen-binding molecules with increased concentration, exposure and/or retention in brain; methods for improving concentration, exposure and/or retention in brain of an antigen-binding molecules; and methods for producing and screening for antigen-binding molecules with increased concentration, exposure and/or retention in brain.

In one aspect, the present disclosure relates to an antigen-binding molecule comprising a first antigen-binding domain and a second antigen-binding domain, wherein the first antigen-binding domain specifically binds a first target that facilitates transfer of the antigen-binding molecule into a mammalian brain (through the blood brain barrier), and the second antigen-binding domain specifically binds a second target wherein the second target (i) is expressed on the cell membrane of brain cells, or (ii) is a brain extracellular matrix (ECM) protein or a brain ECM polysaccharide. The present disclosure further relates to an antigen-binding molecule, wherein the first target is a molecule that is expressed on vascular endothelial cells of the blood-brain barrier (BBB). The disclosure further relates to methods for producing the antigen-binding molecule, a pharmaceutical composition comprising the antigen-binding molecule, a nucleic acid or two or more nucleic acids encoding the antigen-binding molecule, a vector or two or more vectors comprising the nucleic acid or two or more nucleic acids, a host cell comprising (i) the nucleic acid or two or more nucleic acids or; (ii) the vector or two or more vectors, a method for producing the antigen-binding molecule and methods of screening, a method for increasing the concentration of an antigen-binding molecule in the brain of a subject in need thereof, a method for increasing the exposure of an antigen-binding molecule in the brain of a subject in need thereof and method for the retention of an antigen-binding molecule in the brain of a subject in need thereof, and methods for treating a subject and medical uses.

### BACKGROUND ART

Biologics such as antibodies offer the advantages of high specificity, potency and lower off-target toxicity, however, delivery of these drugs to the brain remains the biggest challenge for the development of therapeutics to treat diseases of the central nervous system. The main obstacle is the blood-brain barrier (BBB), which impedes the entrance of most molecules present in the systemic circulation, especially large molecule drugs. Due to the existence of the BBB, it is difficult to obtain an effective concentration at the time of drug administration in the central nervous system, which makes drug development difficult.

Various delivery technologies are being studied to increase the concentration of macromolecular substances such as biologics in the brain. Most of the reported techniques use receptor-mediated transcytosis (RMT), and the receptor expressed in the brain vascular endothelium to serve as a target comprises, for example, a transferrin receptor, an insulin receptor, an insulin-like growth factor receptor (IGFR), a low-density lipoprotein receptor family (LDLRf), and the like. For example, a technology for crossing BBB via a transferrin receptor has been reported by producing a fusion protein of an anti-transferrin receptor antibody and a nerve growth factor. Other technologies using anti-transferrin receptor antibodies include bispecific antibodies of an anti-transferrin receptor antibody and an anti-beta secretase (BACE1) antibody, and other bispecific antibodies (WO 2016/081640 (PTL 1), WO2015/191934 (PTL 2), WO 2016/081643 (PTL 3), and fusion antibodies obtained by fusing a monovalent anti-transferrin receptor antibody to the carboxyl-terminal side of an anti-amyloid beta antibody (WO 2014/033074 (PTL 4)) have been reported. However, transferrin receptor and insulin receptor are expressed not only in the brain vascular endothelial cells but also broadly express throughout the body and non-brain tissue, and therefore, a drug is delivered also to the non-brain tissues. Because the antigen is expressed in the whole body, the half-life of the antibody in the blood is short. As a result, the BBB transfer technologies such as fusion of anti-transferrin receptor binding antibody or anti-insulin receptor antibody have the drawback of fast clearance from systemic circulation (Sci Transl Med. 2013; 5: 183ra57. pmid:23636093 (NPL 1)).

### CITATION LIST

### Patent Literature

[PTL 1] WO 2016/081640
[PTL 2] WO 2015/191934
[PTL 3] WO 2016/081643
[PTL 4] WO 2014/033074

### Non Patent Literature

[NPL 1] Sci Transl Med. 2013; 5: 183ra57. pmid:23636093

### SUMMARY OF INVENTION

### Technical Problem

There is an ongoing need for providing compounds, compositions, methods and uses which are useful in the context of brain diseases or brain disorders. An object of the present disclosure is, without limitation, to provide compounds, compositions, methods and uses which are useful in the context of brain diseases or brain disorders.

### Solution to Problem

As a means for solving the problem, one aspect of the present disclosure provides to an antigen-binding molecule comprising a first antigen-binding domain and a second antigen-binding domain, wherein the first antigen-binding domain specifically binds a first target that facilitates transfer of the antigen-binding molecule into a mammalian brain, and the second antigen-binding domain specifically binds a second target wherein the second target (i) is expressed on the cell membrane of brain cells, or (ii) is a brain extracellular matrix (ECM) protein or a brain ECM polysaccharide, and methods of using the same.

Throughout this disclosure, various aspects of the present invention are provided. However, it should be understood that each of the aspects disclosed is merely exemplary embodiments of the invention and they are not intended to inflexibly limit the invention to these embodiments. On the contrary, the invention is intended to encompass alternatives, modifications and equivalents, which may be included within the spirit and scope of the disclosures.
[1] In a first aspect, an antigen-binding molecule comprising a first antigen-binding domain and a second antigen-binding domain, is provided
wherein the first antigen-binding domain specifically binds a first target that facilitates transfer of the antigen-binding molecule into a mammalian brain, and the second antigen-binding domain specifically binds a second target wherein the second target (i) is expressed on the cell membrane of brain cells, or (ii) is a brain extracellular matrix (ECM) protein or a brain ECM polysaccharide.

### Moreover, the Following Embodiments are Provided:

[2] The antigen-binding molecule of [1], wherein the first target is a molecule that is expressed on vascular endothelial cells of the blood-brain barrier (BBB).
[2A] The antigen-binding molecule of [1] or [2], wherein the first target facilitates transfer of the antigen-binding molecule into a mammalian brain through the BBB.
[3] The antigen-binding molecule of [1],[2] or [2A], wherein the first target is selected from the group consisting of Transferrin receptor (TfR), Basigin (CD147), Glut1, Ldlrad3, CD320, Insulin receptor, insulin-like growth factor 1 receptor (IGF1R), Low density lipoprotein Receptor (LDLR), Low density lipoprotein receptor related protein (LRP), preferably LRP1, Diphtheria toxin Receptor, Glucose receptor, CD98hc, TMEM30A, Leptin receptor (LepR) and heparan sulfate chains branching from proteoglycan (HSPG).
[4] The antigen-binding molecule of any one of [1] to [3], wherein the second target is selected from the group consisting of Myelin Oligodendrocyte glycoprotein (MOG), Neuroglycan C/Chondroitin sulfate proteoglycan 5 (CSPG5), IGSF4B/SynCAM3/Cell Adhesion Molecule 3 (CADM3), CNPase (2′,3′-cyclic nucleotide 3′-phosphodiesterase), Myelin-associated Glycoprotein (MAG), Myelin Basic Protein (MBP), EAAT1 (Solute Carrier Family 1 member 3), EAAT2 (Solute Carrier Family 1 member 2), MAP2 (Microtubule-associated protein2), NEFL (Neurofilament light polypeptide), NEFM (Neurofilament medium polypeptide), NSE (Gamma-enolase), CD68 (Macrosialin), Allograft inflammatory factor 1 (IBA1 or AIF1), Purinergic receptor (P2RY12), Interleukin 1 receptor accessory protein like 1 (ILIRAPL1), Glutamate ionotropic receptor NMDA type subunit 2B (GRIN2B), Calcium voltage-gated channel auxiliary subunit gamma 8 (CACNG8), CD11b (Integrin subunit alpha M), SLC6A2 (Sodium-dependent noradrenaline transporter), DPP6 (Dipeptidyl peptidase like 6), SLC18A3 (Vesicular acetylcholine transporter), Sodium/potassium-transporting ATPase subunit alpha-2, Broad substrate specificity ATP-binding cassette transporter ABCG2, Solute carrier family 12 member 9, Electrogenic sodium bicarbonate cotransporter 1, Excitatory amino acid transporter 2, Chondroitin sulfate proteoglycan 4, Immunoglobulin superfamily DCC subclass member 4, Vang-like protein 2, Neural cell adhesion molecule 1 (N-CAM-1), Low-density lipoprotein receptor-related protein 4 (LRP-4), Phosphoprotein associated with glycosphingolipid-enriched microdomains 1 (Csk-binding protein), Plasma membrane calcium-transporting ATPase 1, Prominin-1, Somatostatin receptor type 1, Carnitine O-palmitoyltransferase 1 brain isoform (CPT1-B), Epidermal growth factor receptor, Protein MAL2, Syntaxin-1A, Sodium/calcium exchanger 1, Lysophosphatidylcholine acyltransferase 1 (LPC acyltransferase 1), Calsyntenin-3 (Alcadein-beta), Pituitary adenylate cyclase-activating polypeptide type I receptor (PACAP type I receptor), Neutral cholesterol ester hydrolase 1 (NCEH), CD166 antigen (Activated leukocyte cell adhesion molecule), Inactive tyrosine-protein kinase 7, Claudin-11, Ectonucleotide phosphatase (ENPP6), Tetraspanin-2 (Tspan-2), Myelin proteolipid protein (PLP), Glycolipid transfer protein (GLTP), Versican core protein (Chondroitin sulfate proteoglycan 2 or CSPG2), Tropoelastin (Elastin), Collagen alpha-2(IV) chain (Canstatin), Proteoglycan link protein 1 (Hyaluronan and proteoglycan link protein 1), Tenascin-R (TN-R), Proteoglycan link protein 2 (Hyaluronan and proteoglycan link protein 2), Collagen alpha-1(I) chain, Neurofilament-3 (NEF3), Immunoglobulin superfamily member 8 (IgSF8), Laminin subunit gamma-1 (LAMC1), Collagen alpha-1(VI) chain (Col6a1), and Collagen alpha-3(VI) chain (Col6a3).
[5] The antigen-binding molecule of any one of [1] to [4], wherein the brain cells comprise one or more of the cells selected from the group consisting of oligodendrocytes, astrocytes, neurons and microglia.
[6] The antigen-binding molecule of any one of [1] to [5], wherein the second target is a molecule that is predominantly expressed on brain-specific cell(s).
[6A] The antigen-binding molecule of any one of [1] to [6], wherein the second target is a molecule that is broadly expressed in brain tissue(s).
[16B] The antigen-binding molecule of [6A], wherein the second target that is broadly expressed in brain tissue(s) is a molecule that is expressed in more than one, more than two, or more than three brain tissues selected from the group consisting of cerebral cortex, hippocampal formation, amygdala, basal ganglia, thalamus, hypothalamus, midbrain, cerebellum, pons, medulla oblongata, and spinal cord.
[6C] The antigen-binding molecule of [16B], wherein the second target is a molecule selected from the group consisting of Interleukin 1 receptor accessory protein like 1 (IL1RAPL1), Glutamate ionotropic receptor NMDA type subunit 2B (GRIN2B), and Calcium voltage-gated channel auxiliary subunit gamma 8 (CACNG8).
[16D] The antigen-binding molecule of any one of [1] to [4], wherein the second target is a brain ECM protein.
[6E] The antigen-binding molecule of [16D], wherein the second target is selected from the group consisting of Versican core protein (Chondroitin sulfate proteoglycan 2 or CSPG2), Tropoelastin (Elastin), Collagen alpha-2(IV) chain (Canstatin), Proteoglycan link protein 1 (Hyaluronan and proteoglycan link protein 1), Tenascin-R (TN-R), Proteoglycan link protein 2 (Hyaluronan and proteoglycan link protein 2), Collagen alpha-1(I) chain, Neurofilament-3 (NEF3), Immunoglobulin superfamily member 8 (IgSF8), Laminin subunit gamma-1 (LAMC1), Collagen alpha-1(VI) chain (Col6a1), and Collagen alpha-3(VI) chain (Col6a3).
[6F] The antigen-binding molecule of any one of [1] to [5], wherein the second target is only expressed on the cell membrane of brain-specific cell(s).
[6G] The antigen-binding molecule of [1] to [5], wherein the second target is only expressed in brain tissue(s).
[6H] The antigen-binding molecule of [1] to [3], wherein the second target is a brain ECM polysaccharide.
[7] The antigen-binding molecule of any one of [1] to [6H], wherein the first target is selected from the group consisting of Transferrin receptor (TfR), Basigin (CD147), Insulin receptor, insulin-like growth factor 1 receptor (IGF1R), Low density lipoprotein Receptor (LDLR), Low density lipoprotein receptor related protein (LRP), preferably LRP1, Diphtheria toxin Receptor, Glucose receptor, preferably Glut1, and CD98hc.
[7A] The antigen-binding molecule of any one of [1] to [7], wherein the first target is Transferrin receptor (TfR), wherein optionally the antigen-binding domain that binds TfR comprises a heavy chain variable region (VH) having the amino acid sequence shown in SEQ ID NO: 29 and/or a light chain variable region (VL) having the amino acid sequence shown in SEQ ID NO: 30, or a variant thereof having one or more conservative amino acid substitutions.
[7B] The antigen-binding molecule of any one of [1] to [7], wherein the first target is Basigin (CD147), wherein optionally the antigen-binding domain that binds Basigin comprises a heavy chain variable region (VH) having the amino acid sequence shown in SEQ ID NO: 31 and/or a light chain variable region (VL) having the amino acid sequence shown in SEQ ID NO: 32, or a variant thereof having one or more conservative amino acid substitutions.
[7C] The antigen-binding molecule of any one of [1] to [7], wherein the first target is Insulin receptor.
[7D] The antigen-binding molecule of any one of [1] to [7], wherein the first target is Low density lipoprotein Receptor (LDLR).
[7E] The antigen-binding molecule of any one of [1] to [7], wherein the first target is Low density lipoprotein receptor related protein (LRP), preferably LRP1.
[7F] The antigen-binding molecule of any one of [1] to [7], wherein the first target is Diphtheria toxin Receptor.
[7G] The antigen-binding molecule of any one of [1] to [7], wherein the first target is Glucose receptor, preferably Glut1.
[7H] The antigen-binding molecule of any one of [1] to [7], wherein the first target is CD98hc.
[7I] The antigen-binding molecule of any one of [1] to [7], wherein the first target is insulin-like growth factor 1 receptor (IGF1R).
[8] The antigen-binding molecule of any one of [1] to [7I], wherein the second target is selected from the group consisting of Myelin Oligodendrocyte glycoprotein (MOG), Neuroglycan C/Chondroitin sulfate proteoglycan 5 (CSPG5), and Cell Adhesion Molecule 3 (CADM3).
[8A] The antigen-binding molecule of any one of [1] to [8], wherein the second target is MOG, wherein optionally the antigen-binding domain that binds MOG comprises a heavy chain variable region (VH) having the amino acid sequence shown in SEQ ID NO: 15 or 34, and/or a light chain variable region (VL) having the amino acid sequence shown in SEQ ID NO: 17 or 35, or a variant thereof having one or more conservative amino acid substitutions.
[8B] The antigen-binding molecule of [8A], wherein the antigen-binding molecule has increased retention in central nerve tissues such as optic nerve, spinal cord, olfactory bulb and medulla oblongata.
[8C] The antigen-binding molecule of any one of [1] to [8], wherein the second target is CSPG5, wherein optionally the antigen-binding domain that binds CSPG5 comprises a heavy chain variable region (VH) having the amino acid sequence shown in SEQ ID NO: 36 or 56, and/or a light chain variable region (VL) having the amino acid sequence shown in SEQ ID NO: 37 or 57, or a variant thereof having one or more conservative amino acid substitutions.
[8D] The antigen-binding molecule of any one of [1] to [8], wherein the second target is CADM3, wherein optionally the antigen-binding domain that binds CADM3 comprises a heavy chain variable region (VH) having the amino acid sequence shown in SEQ ID NO: 38, and/or a light chain variable region (VL) having the amino acid sequence shown in SEQ ID NO: 39, or a variant thereof having one or more conservative amino acid substitutions.
[9] The antigen-binding molecule of any one of [1] to [8C], wherein:
   (i) the first target is Transferrin receptor (TfR) and the second target is Myelin Oligodendrocyte glycoprotein (MOG); or
   (ii) the first target is Transferrin receptor (TfR) and the second target is Neuroglycan C/Chondroitin sulfate proteoglycan 5 (CSPG5); or
   (iii) the first target is Transferrin receptor (TfR) and the second target is Cell Adhesion Molecule 3 (CADM3); or
   (iv) the first target is Basigin (CD147) and the second target is Myelin Oligodendrocyte glycoprotein (MOG); or
   (v) the first target is Basigin (CD147) and the second target is Neuroglycan C/Chondroitin sulfate proteoglycan 5 (CSPG5); or
   (vi) the first target is Basigin (CD147) and the second target is Cell Adhesion Molecule 3 (CADM3); or
   (vii) the first target is Insulin receptor and the second target is Myelin Oligodendrocyte glycoprotein (MOG); or
   (viii) the first target is Insulin receptor and the second target is Neuroglycan C/Chondroitin sulfate proteoglycan 5 (CSPG5); or
   (ix) the first target is Insulin receptor and the second target is Cell Adhesion Molecule 3 (CADM3); or
   (x) the first target is glucose receptor and the second target is Myelin Oligodendrocyte glycoprotein (MOG); or
   (xi) the first target is glucose receptor and the second target is Neuroglycan C/Chondroitin sulfate proteoglycan 5 (CSPG5); or
   (xii) the first target is glucose receptor and the second target is Cell Adhesion Molecule 3 (CADM3); or
   (xiii) the first target is Low density lipoprotein Receptor (LDLR) and the second target is Myelin Oligodendrocyte glycoprotein (MOG); or
   (xiv) the first target is Low density lipoprotein Receptor (LDLR) and the second target is Neuroglycan C/Chondroitin sulfate proteoglycan 5 (CSPG5); or
   (xv) the first target is Low density lipoprotein Receptor (LDLR) and the second target is Cell Adhesion Molecule 3 (CADM3); or
   (xvi) the first target is Low density lipoprotein receptor related protein (LRP) and the second target is Myelin Oligodendrocyte glycoprotein (MOG); or
   (xvii) the first target is Low density lipoprotein receptor related protein (LRP) and the second target is Neuroglycan C/Chondroitin sulfate proteoglycan 5 (CSPG5); or
   (xviii) the first target is Low density lipoprotein receptor related protein (LRP) and the second target is Cell Adhesion Molecule 3 (CADM3); or
   (xix) the first target is CD98hc and the second target is Myelin Oligodendrocyte glycoprotein (MOG); or
   (xx) the first target is CD98hc and the second target is Neuroglycan C/Chondroitin sulfate proteoglycan 5 (CSPG5); or
   (xxi) the first target is CD98hc and the second target is Cell Adhesion Molecule 3 (CADM3).
[10] The antigen-binding molecule of any one of [1] to [4], wherein the second target is not a target selected from the group consisting of beta-secretase 1 (BACE1), Abeta, epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, TRK A, TRK B, TRK C, an alpha synuclein, a beta synuclein, a gamma synuclein, vascular endothelial growth factor (VEGF), neuropilin, a Semaphorin, Semaphorin 3A, Semaphorin 4A, Semaphorin 6A, myelin basic protein (MBP), MOG, PLP, MAG, aquaporin 4, glutamate receptor, and EpCAM.
[11] The antigen-binding molecule of any one of [1] to [10], further comprising
   (i) at least one functional moiety comprising an enzyme, a therapeutic protein, an antibody or antigen-binding fragment thereof, a peptide, a DNA, an shRNA, an siRNA, a small molecule drug, or a cytotoxic agent, and/or
   (ii) at least one in vivo half-life extension moiety, preferably wherein said at least one in vivo half-life extension moiety is selected from the group consisting of an Fc region, an albumin-binding domain, an FcRn-binding protein, an FcRn-binding peptide and a PEG moiety.
[11A] The antigen-binding molecule of [1], wherein the at least one functional moiety is selected from the group consisting of neprilysin, anti-sortilin1 antigen-binding domain, anti-BACE1 antigen-binding domain and acid alpha-glucosidase (GAA).
[12] In a second aspect, a method for increasing the concentration of an antigen-binding molecule in the brain of a subject in need thereof, is provided, the method comprising:
(a) providing a first antigen-binding molecule comprising:
(a1) a first antigen-binding domain that specifically binds a first target that facilitates transfer of the antigen-binding molecule into a mammalian brain; or
(a2) a second antigen-binding domain that specifically binds a second target wherein the second target (i) is expressed on the cell membrane of brain cells, or (ii) is a brain ECM protein or a brain ECM polysaccharide;
(b) introducing
in the case of (a1), to the first antigen-binding molecule at least one second antigen-binding domain that specifically binds the second target wherein the second target (i) is expressed on the cell membrane of brain cells, or (ii) is a brain ECM protein or a brain ECM polysaccharide, thereby obtaining a second antigen-binding molecule; or
in the case of (a2), to the first antigen-binding molecule at least one first antigen-binding domain that specifically binds the first target that facilitates transfer of the antigen-binding molecule into a mammalian brain, thereby obtaining a second antigen-binding molecule;
such that the concentration of said second antigen-binding molecule in the brain of said subject is increased upon administration to said subject as compared to the first antigen-binding molecule.
   Moreover, the following embodiments are provided:
[13] The method of [12], further comprising step (c):
(c) determining that the concentration of said second antigen-binding molecule in the brain of said subject is increased compared to a control antigen-binding molecule, wherein the control antigen-binding molecule differs from the second antigen-binding molecule according to (b) only in that it does not comprise:
in the case of (a1), said at least one second antigen-binding domain that specifically binds the second target wherein the second target (i) is expressed on the cell membrane of brain cells, or (ii) is a brain ECM protein or a brain ECM polysaccharide; or
in the case of (a2) said at least one first antigen-binding domain that specifically binds the first target that facilitates transfer of the antigen-binding molecule into a mammalian brain.
[14] The method of [12] or [13], wherein the concentration of said second antigen-binding molecule in the brain of said subject is Cmax.
[15] In a third aspect, a method for increasing exposure of an antigen-binding molecule in the brain of a subject in need thereof, is provided, the method comprising:
(a) providing a first antigen-binding molecule comprising:
(a1) a first antigen-binding domain that specifically binds a first target that facilitates transfer of the antigen-binding molecule into a mammalian brain; or
(a2) a second antigen-binding domain that specifically binds a second target wherein the second target (i) is expressed on the cell membrane of brain cells, or (ii) is a brain extracellular matrix (ECM) protein or a brain ECM polysaccharide;
(b) introducing
in the case of (a1), to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule; or
in the case of (a2), to the first antigen-binding molecule at least one first antigen-binding domain that specifically binds a first target that facilitates transfer of the antigen-binding molecule into a mammalian brain, thereby obtaining a second antigen-binding molecule;
such that the exposure of said second antigen-binding molecule in the brain of said subject is increased upon administration to said subject as compared to the first antigen-binding molecule.

### Moreover, the Following Embodiments are Provided:

[16] The method of [15], further comprising step (c):
(c) determining that the exposure of said second antigen-binding molecule in the brain of said subject is increased compared to a control antigen-binding molecule, wherein the control antigen-binding molecule differs from the second antigen-binding molecule according to (b) only in that it does not comprise
in the case of (a1), said at least one second antigen-binding domain that specifically binds the second target wherein the second target (i) is expressed on the cell membrane of brain cells, or (ii) is a brain ECM protein or a brain ECM polysaccharide; or
in the case of (a2) said at least one first antigen-binding domain that specifically binds the first target that facilitates transfer of the antigen-binding molecule into a mammalian brain.
[17] The method of [15] or [16], wherein the exposure of said second antigen-binding molecule in the brain of said subject is the AUC (Area Under Curve) of brain concentration-time profiles of the antigen-binding molecule.
[18] In a fourth aspect, a method for the retention of an antigen-binding molecule in the brain of a subject in need thereof is provided, the method comprising:
(a) providing a first antigen-binding molecule comprising a first antigen-binding domain that specifically binds a first target that facilitates transfer of the antigen-binding molecule into a mammalian brain;
(b) introducing to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule,
such that the retention of said second antigen-binding molecule in the brain of said subject is increased upon administration to said subject as compared to the first antigen-binding molecule.

### Moreover, the Following Embodiments are Provided:

[19] The method of [18], further comprising step (c):
(c) determining that the retention of said second antigen-binding molecule in the brain of said subject is increased compared to a control antigen-binding molecule, wherein the control antigen-binding molecule differs from the second antigen-binding molecule according to (b) only in that it does not comprise said at least one antigen-binding domain that specifically binds the second target.
[20] The method of [18] or [19], wherein the retention of said second antigen-binding molecule in the brain of said subject is the half-life in vivo in the brain.
[20A] The method of [20], wherein the half-life in vivo in the brain of said second antigen-binding molecule is at least 10 days, 12 days, 15 days, 20 days, 30 days, 45 days, 60 days or 90 days.
[20B] The method of [20A], wherein the half-life in vivo in the brain of said second antigen-binding molecule is at least 10 days.
[20C] The method of [20A], wherein the half-life in vivo in the brain of said second antigen-binding molecule is at least 30 days.
[20D] The method of [20A], wherein the half-life in vivo in the brain of said second antigen-binding molecule is at least 60 days.
[21] The antigen-binding molecule of any one of [1] to [11] or the method of any one of [12] to [20D], wherein the mammalian brain is human brain.
[22] The method of any one of [12] to [21], wherein the first target is as specified in any one of [2] to [3] and [7] to [7I].
[23] The method of any one of [12] to [22], wherein the second target is as specified in any one of [4], [5], [6] to [6H], [8] to [8C] and [10].
[24] The method of any one of [12] to [23], wherein the first target and the second target are as specified in any one of [2] to [11].
[25] The method of any one of [12] to [24], wherein:
(i) the first target is Transferrin receptor (TfR) and the second target is Myelin Oligodendrocyte glycoprotein (MOG);
(ii) the first target is Transferrin receptor (TfR) and the second target is Neuroglycan C/Chondroitin sulfate proteoglycan 5 (CSPG5);
(iii) the first target is Transferrin receptor (TfR) and the second target is Cell Adhesion Molecule 3 (CADM3);
(iv) the first target is Basigin (CD147) and the second target is Myelin Oligodendrocyte glycoprotein (MOG);
(v) the first target is Basigin (CD147) and the second target is Neuroglycan C/Chondroitin sulfate proteoglycan 5 (CSPG5); or
(vi) the first target is Basigin (CD147) and the second target is Cell Adhesion Molecule 3 (CADM3);
(vii) the first target is Insulin receptor and the second target is Myelin Oligodendrocyte glycoprotein (MOG);
(viii) the first target is Insulin receptor and the second target is Neuroglycan C/Chondroitin sulfate proteoglycan 5 (CSPG5);
(ix) the first target is Insulin receptor and the second target is Cell Adhesion Molecule 3 (CADM3);
(x) the first target is glucose receptor and the second target is Myelin Oligodendrocyte glycoprotein (MOG);
(xi) the first target is glucose receptor and the second target is Neuroglycan C/Chondroitin sulfate proteoglycan 5 (CSPG5);
(xii) the first target is glucose receptor and the second target is Cell Adhesion Molecule 3 (CADM3);
(xiii) the first target is Low density lipoprotein Receptor (LDLR) and the second target is Myelin Oligodendrocyte glycoprotein (MOG);
(xiv) the first target is Low density lipoprotein Receptor (LDLR) and the second target is Neuroglycan C/Chondroitin sulfate proteoglycan 5 (CSPG5); or
(xv) the first target is Low density lipoprotein Receptor (LDLR) and the second target is Cell Adhesion Molecule 3 (CADM3);
(xvi) the first target is Low density lipoprotein receptor related protein (LRP) and the second target is Myelin Oligodendrocyte glycoprotein (MOG);
(xvii) the first target is Low density lipoprotein receptor related protein (LRP) and the second target is Neuroglycan C/Chondroitin sulfate proteoglycan 5 (CSPG5); or
(xviii) the first target is Low density lipoprotein receptor related protein (LRP) and the second target is Cell Adhesion Molecule 3 (CADM3) or
(xix) the first target is CD98hc and the second target is Myelin Oligodendrocyte glycoprotein (MOG); or
(xx) the first target is CD98hc and the second target is Neuroglycan C/Chondroitin sulfate proteoglycan 5 (CSPG5); or
(xxi) the first target is CD98hc and the second target is Cell Adhesion Molecule 3 (CADM3).
[26] The method of any one of [12] to [25], wherein the second antigen-binding molecule is an antigen-binding molecule according to any one of [1] to [11].
[26A] The method of any one of [15] to [25], wherein when the second target is MOG, said second antigen-binding molecule has increased retention/concentration/exposure in central nerve tissues such as optic nerve, spinal cord, olfactory bulb and medulla oblongata compared to the control antigen-binding molecule.
[27] In a fifth aspect, a pharmaceutical composition is provided comprising the antigen-binding molecule according to any one of [1] to [11] and one or more pharmaceutically acceptable carrier(s) or excipient(s).

### Moreover, the Following Embodiments are Provided:

[28] The antigen-binding molecule according to any one of [1] to [11] or the pharmaceutical composition of [27], for use (i) in a method of increasing the concentration of the antigen-binding molecule in the brain of a subject in need thereof; (ii) in a method of increasing exposure of the antigen-binding molecule in the brain of a subject in need thereof; and/or (iii) for the retention of the antigen-binding molecule in the brain of a subject in need thereof; optionally wherein the subject is a human.
[29] In a sixth aspect, a nucleic acid or two or more nucleic acids encoding the antigen-binding molecule according to any one of [1] to [11] is/are provided, optionally wherein the nucleic acid or two or more nucleic acids are operably linked to a promotor.
[30] In a seventh aspect, a vector or two or more vectors comprising the nucleic acid or two or more nucleic acids according to [29].
[31] In an eighth aspect, a host cell is provided comprising (i) the nucleic acid or two or more nucleic acids of [29]; (ii) the vector or two or more vectors of [30]; and/or (iii) capable of expressing antigen-binding molecule according to any one of [1] to [11].
[32] In a ninth aspect, a method of producing an antigen-binding molecule according to any one of [1] to [11] is provided, comprising culturing the host cell of
[31] so that the antigen-binding molecule is produced; optionally further comprising recovering the antigen-binding molecule from the host cell.

### Moreover, the Following Embodiments are Provided:

[33] The antigen-binding molecule of any one of [1] to [11] and [21], wherein the first antigen-binding domain is an antibody variable region comprising an antibody heavy chain variable region (VH) and an antibody light chain variable region (VL).
[33A] The antigen-binding molecule of any one of [1] to [11] and [21], wherein the first antigen-binding domain is a Fab, Fab′, F(ab′)₂, diabody, triabody, scFab, Fv, scFv, or single-domain antibody (VHH), or a non-antibody binder (e.g. affibody, DARPins, FN3, aptamer, anticalins).
[34] The antigen-binding molecule of any one of [1] to [11], [21], [33] and [33A], wherein the second antigen-binding domain is an antibody variable region comprising an antibody heavy chain variable region (VH) and an antibody light chain variable region (VL).
[34A] The antigen-binding molecule of any one of [1] to [11], [21], [33] and [33A], wherein the second antigen-binding domain is a Fab, Fab′, F(ab′)₂, diabody, triabody, scFab, Fv, scFv, or single-domain antibody (VHH), or a non-antibody binder (e.g. affibody, DARPins, FN3, aptamer, anticalins).
[35] The antigen-binding molecule of any one of [1] to [11], [21], and [33] to [34A], wherein the first antigen-binding domain and the second antigen-binding domain are independently selected from the group consisting of an antibody variable region comprising an antibody heavy chain variable region (VH) and an antibody light chain variable region (VL), and a Fab, Fab′, F(ab′)₂, diabody, triabody, scFab, Fv, scFv, or single-domain antibody (VHH).
[36] The antigen-binding molecule of any one of [1] to [11], [21], and [33] to [35], wherein the antigen-binding molecule contains 1, 2, 3 or 4 first antigen-binding domain(s) and 1, 2 3 or 4 second antigen-binding domain(s).
[37] The antigen-binding molecule of any one of [1] to [11], [21], and [33] to [36], wherein the antigen-binding molecule contains 1, 2, 3 or 4 first antigen-binding domain(s) and 1, 2, 3 or 4 second antigen-binding domains and at least one Fc region.
[38] The antigen-binding molecule of any one of [1] to [11], [21], and [33] to [37], wherein the first antigen-binding domain(s) and the second antigen-binding domain(s) are independently selected from the group consisting of an antibody variable region comprising an antibody heavy chain variable region (VH) and an antibody light chain variable region (VL), a Fab, Fab′, F(ab′)₂, diabody, triabody, scFab, Fv, scFv, or single-domain antibody (VHH), or a non-antibody binder (e.g. affibody, DARPins, FN3, aptamer, anticalins).
[38] The antigen-binding molecule of any one of [1] to [11], [21], and [33] to [37], which is a bispecific antibody.
[39] The antigen-binding molecule of [38], containing 1 first antigen-binding domain and 1 second antigen-binding domain.
[40] The antigen-binding molecule of [38] or [39], wherein 1 first antigen-binding domain and 1 second antigen-binding domain are linked to a Fc region.
[40A] The antigen-binding molecule of [36] or [40], wherein the Fc region is a Fc region of which the ability to bind to the activating Fc gamma receptor is decreased compared to the ability of an Fc region of a native human IgG to bind to the activating Fc gamma receptor.
[40B] The antigen-binding molecule of [40A], wherein the activating Fc gamma receptor is human Fc gamma RIa, human Fc gamma RIIa(R), human Fc gamma RIIa(H), human Fc gamma RIIIa(V), or human Fc gamma RIIIa(F).
[40C] The antigen-binding molecule of [40A] or [40B], wherein the Fc region comprises one or more of the following amino acid substitutions (all positions by EU numbering):
Ala, Arg, Asn, Asp, Gln, Glu, Gly, His, Lys, Met, Phe, Pro, Ser, Thr, or Trp at position 234;
Ala, Asn, Asp, Gln, Glu, Gly, His, Ile, Lys, Met, Pro, Ser, Thr, Val, or Arg at position 235;
Arg, Asn, Gln, His, Leu, Lys, Met, Phe, Pro, or Tyr at position 236; Ala, Asn, Asp, Gln, Glu, His, Ile, Leu, Lys, Met, Pro, Ser, Thr, Val, Tyr, or Arg at position 237;
Ala, Asn, Gln, Glu, Gly, His, Ile, Lys, Thr, Trp, or Arg at position 238; Gln, His, Lys, Phe, Pro, Trp, Tyr, or Arg at position 239;
Ala, Arg, Asn, Gln, Gly, His, Ile, Leu, Lys, Met, Phe, Ser, Thr, Trp, Tyr, or Val at position 265;
Ala, Arg, Asn, Asp, Gln, Glu, Gly, His, Lys, Phe, Pro, Ser, Thr, Trp, or Tyr at position 266;
Arg, His, Lys, Phe, Pro, Trp, or Tyr at position 267;
Ala, Arg, Asn, Gln, Gly, His, Ile, Leu, Lys, Met, Phe, Pro, Ser, Thr, Trp, Tyr, or Val at position 269;
Ala, Arg, Asn, Gln, Gly, His, Ile, Leu, Lys, Met, Phe, Pro, Ser, Thr, Trp, Tyr, or Val at position 270;
Arg, His, Phe, Ser, Thr, Trp, or Tyr at position 271;
Arg, Asn, Asp, Gly, His, Phe, Ser, Trp, or Tyr at position 295;
Arg, Gly, Lys, or Pro at position 296;
Ala at position 297;
Arg, Gly, Lys, Pro, Trp, or Tyr at position 298;
Arg, Lys, or Pro at position 300;
Lys or Pro at position 324;
Ala, Arg, Gly, His, Ile, Lys, Phe, Pro, Thr, Trp, Tyr, or Val at position 325;
Arg, Gln, His, Ile, Leu, Lys, Met, Phe, Pro, Ser, Thr, Trp, Tyr, or Val at position 327;
Arg, Asn, Gly, His, Lys, or Pro at position 328;
Asn, Asp, Gln, Glu, Gly, His, Ile, Leu, Lys, Met, Phe, Ser, Thr, Trp, Tyr, Val, or Arg at position 329;
Pro or Ser at position 330;
Arg, Gly, or Lys at position 331;
Arg, Lys, or Pro at position 332.
[41] The antigen-binding molecule of any one of [1] to [11], [21], and [33] to [40], wherein the at least one functional moiety is an antibody or antigen-binding fragment thereof which specifically binds a membrane protein of i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, ii) a tumor cell, or iii) an autoreactive cell.
[42] The antigen-binding molecule of [41], wherein said antibody or antigen-binding fragment thereof specifically binds a membrane protein selected from the group consisting of a T cell receptor, CD3, CD137, CD40, CTLA4, a costimulatory molecule and a coinhibitory molecule.
[43] In a tenth aspect, a method for producing an antigen-binding molecule is provided, which comprises the steps of:
(a) selecting a first antigen-binding domain that specifically binds a first target that facilitates transfer of the antigen-binding molecule into a mammalian brain;
(b) selecting a second antigen-binding domain that specifically binds a second target wherein the second target (i) is expressed on the cell membrane of brain cells, or (ii) is a brain ECM protein or a brain ECM polysaccharide;
(c) obtaining one or more nucleic acid(s) encoding an antigen-binding molecule in which an antigen-binding domain and an antigen-binding domain prepared in (a) and (b) are linked; and
(d) producing an antigen-binding molecule using the one or more nucleic acid(s) prepared in (c).
[44] In an eleventh aspect, a method for screening an antigen-binding molecule is provided, which comprises the steps of:
(a) selecting a first antigen-binding domain that specifically binds a first target that facilitates transfer of the antigen-binding molecule into a mammalian brain;
(b) selecting a second antigen-binding domain that specifically binds a second target wherein the second target (i) is expressed on the cell membrane of brain cells, or (ii) is a brain ECM protein or a brain ECM polysaccharide;
(c) obtaining one or more nucleic acid(s) encoding an antigen-binding molecule in which an antigen-binding domain and an antigen-binding domain prepared in (a) and (b) are linked; and
(d) producing an antigen-binding molecule using the one or more nucleic acid(s) prepared in (c).

### Moreover, the Following Embodiments are Provided:

[45] The method of [43] or [44], further comprising step (e):
(e) determining that the (i) retention (ii) concentration or (iii) exposure of said antigen-binding molecule according to (d) in the brain of a subject is increased compared to a control antigen-binding molecule, wherein the control antigen-binding molecule differs from the antigen-binding molecule according to (d) only in that it comprises either one but not both of the antigen-binding domains defined in (a) and (b).
[45A] The method of any one of [43] to [45], wherein when the second target is MOG,
the increase of (i) retention (ii) concentration or (iii) exposure of the second antigen-binding molecule in the brain is an increase of (i) retention (ii) concentration or (iii) exposure in the central nerve tissues such as optic nerve, spinal cord, olfactory bulb and medulla oblongata compared to the control antigen-binding molecule.
[46] In a twelfth aspect, a pharmaceutical composition is provided comprising
(i) an antigen-binding molecule of any one of [1] to [11], [21], and [33] to [42],
(ii) a nucleic acid or two or more nucleic acids encoding the antigen-binding molecule according to [29], (iii) a vector or two or more vectors according to [30], or (iv) a host cell is according to [31], and one or more pharmaceutically acceptable carrier(s) or excipient(s).
[47] In a thirteenth aspect, provided herewith is (i) an antigen-binding molecule of any one of [1] to [11], [21], and [33] to [42], (ii) a nucleic acid or two or more nucleic acids encoding the antigen-binding molecule according to [29], (iii) a vector or two or more vectors according to [30], (iv) a host cell is according to [31], or (iv) a pharmaceutical composition according to [27] or [46], for use as a medicament.
[48] In a fourteenth aspect, provided herewith is (i) an antigen-binding molecule of any one of [1] to [11], [21], and [33] to [42], (ii) a nucleic acid or two or more nucleic acids encoding the antigen-binding molecule according to [29], (iii) a vector or two or more vectors according to [30], (iv) a host cell is according to [31], or (iv) a pharmaceutical composition according to [27] or [46], for use as a diagnostic.
[49] In a fifteenth aspect, provided herewith is (i) an antigen-binding molecule of any one of [1] to [11], [21], and [33] to [42], (ii) a nucleic acid or two or more nucleic acids encoding the antigen-binding molecule according to [29], (iii) a vector or two or more vectors according to [30], (iv) a host cell is according to [31], or (iv) a pharmaceutical composition according to [27] or [46], for use in the treatment and/or prevention of a brain disorder or disease in a subject.
[50] In a sixteenth aspect, provided herewith is a method of treating and/or preventing a brain disorder or disease in a subject in need thereof, the method comprising administering to said subject a therapeutically effective amount of (i) an antigen-binding molecule of any one of [1] to [11], [21], and [33] to [42], (ii) a nucleic acid or two or more nucleic acids encoding the antigen-binding molecule according to [29], (iii) a vector or two or more vectors according to [30], (iv) a host cell is according to [31], or (iv) a pharmaceutical composition according to [27] or [46].
[51] In a seventeenth aspect, provided herewith is (i) an antigen-binding molecule of any one of [1] to [11], [21], and [33] to [42], (ii) a nucleic acid or two or more nucleic acids encoding the antigen-binding molecule according to [29], (iii) a vector or two or more vectors according to [30], (iv) a host cell is according to [31], or (iv) a pharmaceutical composition according to [27] or [46], for use in the preparation of a medicament for the treatment and/or prevention of a brain disorder or disease.
[52] In a eighteenth aspect, provided herewith is the use of (i) an antigen-binding molecule of any one of [1] to [11], [21], and [33] to [42], (ii) a nucleic acid or two or more nucleic acids encoding the antigen-binding molecule according to [29], (iii) a vector or two or more vectors according to [30], (iv) a host cell is according to [31], or (iv) a pharmaceutical composition according to [27] or [46], for the preparation of a medicament for the treatment and/or prevention of a brain disorder or disease.
[53] The pharmaceutical composition/antigen-binding molecule/nucleic acid/vector for use of [49] or [51], the method of treating and/or preventing of [50], or the use of [52], wherein the brain disorder or diseases is selected from the group consisting of neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heredodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Hallervorden-Spatz syndrome, Lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), psychiatric disorders, cancer (e.g. of the CNS, including brain metastases resulting from cancer elsewhere in the body).
[54] The pharmaceutical composition/antigen-binding molecule/nucleic acid/vector for use of [49] or [51], the method of treating and/or preventing of [50], or the use of [52], wherein the brain disorder or diseases is selected from the group consisting of Alzheimer's disease, Pompe disease, Frontotemporal dementia (FTD), and Amyotrophic lateral sclerosis (ALS).

Unless explicitly stated otherwise or unless there are inconsistencies in the context, any terms expressed in the singular form herein may be meant to also include the plural form and any terms expressed in the plural form herein may be meant to also include the singular form. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### BRIEF DESCRIPTION OF DRAWINGS

FIG. 1A A schematic drawing showing a molecule having a brain transfer moiety (a first antigen-binding domain that specifically binds a first target that facilitates transfer of the antigen-binding molecule into a mammalian brain; e.g. anti-Transferrin receptor antibody.

FIG. 1B A schematic drawing showing a molecule having a brain retention/targeting moiety (a second antigen-binding domain specifically binds a second target wherein the second target (i) is expressed on the cell membrane of brain cells, or (ii) is a brain ECM protein or a brain ECM polysaccharide; e.g. anti-MOG antibody).

FIG. 1C A schematic drawing showing a molecule having a brain transfer moiety (a first antigen-binding domain that specifically binds a first target that facilitates transfer of the antigen-binding molecule into a mammalian brain; e.g. anti-Transferrin receptor domain) and a brain retention moiety (a second antigen-binding domain specifically binds a second target wherein the second target (i) is expressed on the cell membrane of brain cells, or (ii) is a brain ECM protein or a brain ECM polysaccharide; e.g. anti-MOG domain).

FIG. 2a A schematic drawing is provided in (a) showing the concept of embodiments of the antigen-binding molecules of the present disclosures, which comprise (1) a brain transfer moiety, (2) a brain retention moiety, (3) a functional moiety, and, optionally, in addition, a half-life extension moiety.

FIG. 2b A schematic drawing showing exemplary molecular formats of the antigen-binding molecules of the present aspects and embodiments. In one example, the antigen-binding molecules comprise a first Fab region that binds a molecule that facilitates penetration or transfer of the antigen-binding molecule into brain (i.e. brain-transfer moiety) and a second Fab regions that binds a molecule that is specifically expressed on a brain-specific cell, or is a brain ECM protein or a brain ECM polysaccharide (i.e. brain-retention moiety), and optionally further comprises a functional moiety. The structure of said brain retention moiety and brain transfer moiety is not limited to Fab region, but can also in the form of antibody fragments such as scFab (single chain Fab), Fv, Fab, Fab′, F(ab′)₂, diabody, triabody, scFv, VHH, diabodies, or F(ab′)2 fragments. In other exemplary embodiments, variable domains are linked to an Fc region such that no F(ab′)2 fragments are present. A plurality of such embodiments are depicted in FIG. 2(b) and FIG. 2(c).

FIG. 2c A schematic drawing showing exemplary molecular formats of the antigen-binding molecules of the present aspects and embodiments. See also the brief description of FIG. 2b.

FIG. 3a A drawing showing the pharmacokinetic profiles in brain and brain concentration-time profiles of KLH, MOG303, KLH/TfR and MOG303/TfR. KLH, MOG303, KLH/TfR, and MOG303/TfR were administered intravenously at a dose of 2 mg/kg, and brain was collected after perfusion. The antibody concentrations in brain were measured by ECL. FIG. 3a shows the antibody concentrations in brain. The data show mean+/−SD (n=6).

FIG. 3b A drawing showing the pharmacokinetic profiles in brain and brain concentration-time profiles of KLH, MOG303, KLH/TfR and MOG303/TfR. KLH, MOG303, KLH/TfR, and MOG303/TfR were administered intravenously at a dose of 2 mg/kg, and brain was collected after perfusion. The antibody concentrations in brain were measured by ECL. FIG. 3b shows the ratio of the antibody concentrations in brain to that of in plasma. The data show mean+/−SD (n=6).

FIG. 3c A drawing showing the pharmacokinetic profiles in brain and brain concentration-time profiles of KLH, MOG303, KLH/TfR and MOG303/TfR. KLH, MOG303, KLH/TfR, and MOG303/TfR were administered intravenously at a dose of 2 mg/kg, and brain was collected after perfusion. The antibody concentrations in brain were measured by ECL. FIG. 3c shows percent of injected dose per brain tissue weight (% ID/g brain). The data show mean+/−SD (n=6).

FIG. 3d A drawing showing the pharmacokinetic profiles in brain and brain concentration-time profiles of KLH, MOG303, KLH/TfR and MOG303/TfR. KLH, MOG303, KLH/TfR, and MOG303/TfR were administered intravenously at a dose of 2 mg/kg, and brain was collected after perfusion. The antibody concentrations in brain were measured by ECL. FIG. 3d shows the cumulative brain AUC until day 28. The data show mean+/−SD (n=6).

FIG. 4 A drawing showing plasma concentration-time profiles of the antibodies. KLH, MOG303, KLH/TfR, and MOG303/TfR were administered intravenously to mice at a dose of 2 mg/kg, and blood was collected. The antibody concentrations in plasma were measured by ECL. The data show mean+/−SD (n=6).

FIG. 5 A drawing showing plasma concentration-time profiles of the antibodies KLH, mTfR/KLH, mBsg/KLH, KLH/MOG303, KLH/MOG307, KLH/CADM3, mTfR/MOG303, mTfR/MOG307, mTfR/CADM3, mBsg/MOG303 and mBsg/MOG307. The antibodies were administered intravenously at a dose of 2 mg/kg, and blood was collected. The antibody concentrations in plasma were measured by ECL. The data show mean+/−SD (n=3-4).

FIG. 6 A drawing showing the pharmacokinetic profiles in brain and brain concentration-time profiles of the antibodies KLH, mTfR/KLH, mBsg/KLH, KLH/MOG303, KLH/MOG307, KLH/CADM3, mTfR/MOG303, mTfR/MOG307, mTfR/CADM3, mBsg/MOG303, and mBsg/MOG307. The antibodies were administered intravenously at a dose of 2 mg/kg, and brain was collected after perfusion. The antibody concentrations in brain were measured by ECL. (a) The antibody concentrations in brain. (b) The ratio of the antibody concentrations in brain to those in plasma. (c) Percent of injected dose per brain tissue weight (% ID/g brain). The data show mean+/−SD (n=3-4).

FIG. 7 A drawing showing plasma concentration-time profiles of the antibodies KLH, mBsg/KLH, mBsg/MOG303, mBsg/CSPG5.2 and mBsg/CADM3. The antibodies were administered intravenously at a dose of 2 mg/kg, and blood was collected. The antibody concentrations in plasma were measured by ECL. The data show mean+/−SD (n=4).

FIG. 8 A drawing showing the pharmacokinetic profiles in brain and brain concentration-time profiles of the antibodies KLH, mBsg/KLH, mBsg/MOG303, mBsg/CSPG5.2 and mBsg/CADM3. The antibodies were administered intravenously at a dose of 2 mg/kg, and brain was collected after perfusion. The antibody concentrations in brain were measured by ECL. (a) The antibody concentrations in brain. (b) The ratio of the antibody concentrations in brain to those in plasma. (c) Percent of injected dose per brain tissue weight (% ID/g brain). The data show mean+/−SD (n=4).

FIG. 9 A drawing showing plasma concentration-time profiles of the antibodies KLH, CSPG5120 and CSPG5120-BS. The antibodies were administered intravenously at a dose of 2 mg/kg, and blood was collected. The antibody concentrations in plasma were measured by ECL. The data show mean+/−SD (n=3).

FIG. 10 A drawing showing the pharmacokinetic profiles in brain and brain concentration-time profiles of the antibodies KLH, CSPG5120 and CSPG5120-BS. The antibodies were administered intravenously at a dose of 2 mg/kg, and brain was collected after perfusion. The antibody concentrations in brain were measured by ECL. (a) The antibody concentrations in brain. (b) The ratio of the antibody concentrations in brain to those in plasma. (c) Percent of injected dose per brain tissue weight (% ID/g brain). The data show mean+/−SD (n=3).

FIG. 11 A schematic drawing showing additional exemplary molecular formats of the antigen-binding molecules of the present aspects and embodiments. FIG. 11A shows schematic structure of the antibodies MOG303/TfR and CSPG5120-BS exemplified in Example 2 and Example 4B respectively, which comprises a first Fab region that binds a molecule that facilitates penetration or transfer of the antigen-binding molecule into brain (i.e. brain-transfer moiety e.g. TfR-binding domain or basigin-binding domain), and a second and a third Fab regions that each binds a molecule that is specifically expressed on a brain-specific cell, or is a brain ECM protein or a brain ECM polysaccharide (i.e. brain-retention moiety, e.g. MOG-binding domain or CSPG5-binding domain), and optionally further comprises a functional moiety (not shown in the figure). FIG. 11B shows schematic structure of the bispecific antibodies (f) to (m) exemplified in Example 4A, which comprises a first Fab region that binds a molecule that facilitates penetration or transfer of the antigen-binding molecule into brain (i.e. brain-transfer moiety e.g. TfR-binding domain or basigin-binding domain), and a second Fab region that binds a molecule that is specifically expressed on a brain-specific cell, or is a brain ECM protein or a brain ECM polysaccharide (i.e. brain-retention moiety, e.g. MOG-binding domain, CSPG5-binding domain, CADM3-binding domain), and optionally further comprises a functional moiety (not shown in the figure). FIG. 11C to 11F show additional exemplary molecular formats of the antigen-binding molecules which additionally comprises one or more functional moiety. The structure of said brain retention moiety and brain transfer moiety is not limited to Fab region, but can also in the form of antibody fragments such as scFab (single chain Fab), Fv, Fab, Fab′, F(ab′)₂, diabody, triabody, scFv, VHH, diabodies, F(ab′)2 fragments, or non-antibody binder (e.g. affibody, DARPins, FN3, aptamer, anticalins). The functional moiety can be any molecule that has therapeutic function such as agonist, antagonist, enzyme, modulator, stabilizer, cell death inducer and any molecular format such as nucleic acid, small molecule, cyclic peptide, peptide, ligand, cytokine, chemokine, growth factor, enzyme and antigen binding domain and so on. In some examples, the functional moiety is selected from the group consisting of neprilysin, anti-sortilin1 antigen-binding domain, anti-BACE1 antigen-binding domain and acid alpha-glucosidase (GAA).

FIG. 12 A schematic drawing showing additional exemplary molecular formats of the antigen-binding molecules of the present aspects and embodiments. FIG. 12A shows schematic structure of the antibodies IGF1R/MOG303 and TfRVNAR.CloneC/MOG303 exemplified in Example 8, which each comprises a VHH that binds a molecule that facilitates penetration or transfer of the antigen-binding molecule into brain (i.e. brain-transfer moiety e.g. TfR-binding domain or IGF1R-binding domain), and a Fab region that binds a molecule that is specifically expressed on a brain-specific cell, or is a brain ECM protein or a brain ECM polysaccharide (i.e. brain-retention moiety, e.g. MOG-binding domain), and optionally further comprises a functional moiety (not shown in the figure).

FIG. 12B shows schematic structure of IL6R/MOG303-TfR-GAA or IL6R/MOG303-TfR-shortGAA exemplified in Example 9A, which comprises a scFab that binds a molecule that facilitates penetration or transfer of the antigen-binding molecule into brain (i.e. brain-transfer moiety e.g. TfR-binding domain), a Fab that binds a molecule that is specifically expressed on a brain-specific cell, or is a brain ECM protein or a brain ECM polysaccharide (i.e. brain-retention moiety, e.g. MOG-binding domain), and one or more functional moiety (e.g. GAA or NEP), and optionally a VHH that binds to another antigen.

FIG. 12C shows schematic structure of MOG303-TfR-2GAA or MOG303-TfR-2shortGAA exemplified in Example 9A, which comprises two Fabs that each binds a molecule that is specifically expressed on a brain-specific cell, or is a brain ECM protein or a brain ECM polysaccharide (i.e. brain-retention moiety, e.g. MOG-binding domain), a scFab that binds a molecule that facilitates penetration or transfer of the antigen-binding molecule into brain (i.e. brain-transfer moiety e.g. TfR-binding domain), and one or more (e.g. two) functional moiety (e.g. GAA) linked to the C terminal of each of the two light chains of the Fab(s) of brain-retention moiety.

FIG. 12D shows schematic structure of BACE1/MOG303-TfR exemplified in Example 9C, which comprises a scFab that binds a molecule that facilitates penetration or transfer of the antigen-binding molecule into brain (i.e. brain-transfer moiety e.g. TfR-binding domain), a Fab that binds a molecule that is specifically expressed on a brain-specific cell, or is a brain ECM protein or a brain ECM polysaccharide (i.e. brain-retention moiety, e.g. MOG-binding domain), and a functional moiety which is a Fab that binds to another biological target (e.g. BACE1 binding domain).

The structure of said brain retention moiety and brain transfer moiety is not limited to Fab, VHH or scFab, but can also in the form of antibody fragments such as scFab (single chain Fab), Fv, Fab, Fab′, F(ab′)₂, diabody, triabody, scFv, VHH, diabodies, F(ab′)2 fragments, or non-antibody binder (e.g. affibody, DARPins, FN3, aptamer, anticalins). The functional moiety can be any molecule that has therapeutic function such as agonist, antagonist, enzyme, modulator, stabilizer, cell death inducer and any molecular format such as nucleic acid, small molecule, cyclic peptide, peptide, ligand, cytokine, chemokine, growth factor, enzyme and antigen binding domain and so on. In some examples, the functional moiety is selected from the group consisting of neprilysin, anti-sortilin1 antigen-binding domain, anti-BACE1 antigen-binding domain and acid alpha-glucosidase (GAA).

FIG. 13 A schematic drawing showing additional exemplary molecular formats of the antigen-binding molecules of the present aspects and embodiments, which are exemplified in Example 9D. FIG. 13A shows schematic structure of bivalent anti-Sort1 antibody (Sort1). FIG. 13B shows schematic structure of Sort1-TfR, which comprises two Fabs that each bind to Sort1 and a scFab that binds to TfR. FIG. 13C is schematic structure of Sort1-TfR-MOG303(L) which comprises two Fabs that each bind to a biological target e.g. Sort1 (functional moiety), a scFab that binds a molecule that facilitates penetration or transfer of the antigen-binding molecule into brain (i.e. brain-transfer moiety e.g. TfR-binding domain), and two scFvs that each binds to a molecule that is specifically expressed on a brain-specific cell, or is a brain ECM protein or a brain ECM polysaccharide (i.e. brain-retention moiety, e.g. anti-MOG scFvs), wherein each of the two brain-retention moieties (anti-MOG scFv) is linked at the C terminal of L chain of anti-SORT1 Fabs. FIG. 13D is schematic structure of Sort1-TfR-MOG303(H) which comprises two Fabs that each bind to a biological target e.g. Sort1 (functional moiety), a scFab that binds a molecule that facilitates penetration or transfer of the antigen-binding molecule into brain (i.e. brain-transfer moiety e.g. TfR-binding domain), and two scFvs that each binds to a molecule that is specifically expressed on a brain-specific cell, or is a brain ECM protein or a brain ECM polysaccharide (i.e. brain-retention moiety, e.g. anti-MOG scFvs), wherein each of the two brain-retention moieties (anti-MOG scFv) is linked at the C terminal of H chain of anti-SORT1 antibody.

FIG. 14 A diagram shows tissue concentrations of the antibodies KLH, MOG303, KLH/TfR and MOG303/TfR tested in Example 10. The antibodies were administered intravenously at a dose of 10 mg/kg, and plasma, brain, liver, muscle, spleen and lung were collected. The antibody concentrations in these tissues were measured by ECL. The data show mean+/−SD (n=3-4).

FIG. 15 A diagram shows tissue concentrations of the antibodies KLH, MOG303, KLH/TfR and MOG303/TfR tested in Example 10. The antibodies were administered intravenously at a dose of 10 mg/kg, and optic nerve, spinal cord, olfactory bulb, retinal and medulla oblongata were collected. The antibody concentrations in these tissues were measured by ECL. The data show mean+/−SD (n=3-4).

FIG. 16 A diagram shows the antibody concentrations in plasma of (a) IGF1R/KLH, IGF1R/MOG303, (b) TfRVNAR.CloneC/KLH, TfRVNAR.CloneC/MOG303, and control antibodies. The antibodies were administered intravenously at a dose of 2 mg/kg, and blood was collected. The antibody concentrations in plasma were measured by ECL. The data show mean+/−SD (n=3).

FIG. 17 A diagram shows the antibody concentrations in brain of (a) IGF1R/KLH, IGF1R/MOG303, (b) TfRVNAR.CloneC/KLH, TfRVNAR.CloneC/MOG303, and control antibodies. The antibodies were administered intravenously at a dose of 2 mg/kg, and brain was collected after perfusion. The antibody concentrations in brain were measured by ECL. The data show mean+/−SD (n=3).

FIG. 18(a) shows the plasma concentrations of the anti-Sortilin1 antibodies (Sort1, Sort1-TfR, Sort1-TfR-MOG303(L) and Sort1-TfR-MOG303(H)) and negative control antibody (KLH). The antibodies were administered intravenously at a dose of 200 nmol/kg, and blood was collected. The antibody concentrations in plasma were measured by ECL. The data show mean+/−SD (n=5). FIG. 18b shows the brain concentrations of the anti-Sortilin1 antibodies (Sort1, Sort1-TfR, Sort1-TfR-MOG303(L) and Sort1-TfR-MOG303(H)) and control antibody (KLH). The antibodies were administered intravenously at a dose of 200 nmol/kg, and brain was collected after perfusion. The antibody concentrations in brain were measured by ECL. The data show mean+/−SD (n=4-5).

FIG. 19(a) shows the plasma concentration-time profiles of the antibodies KLH, IL6R/KLH-NEP, IL6R/KLH-TfR-NEP, and IL6R/MOG303-TfR-NEP which were administered intravenously to mice at a dose of 50 nmol/kg, and blood was collected. The antibody concentrations in plasma were measured by ECL. The data show mean+/−SD (n=6).

FIG. 19(b) shows the brain concentration-time profiles of the antibodies KLH, IL6R/KLH-NEP, IL6R/KLH-TfR-NEP and IL6R/MOG303-TfR-NEP which were administered intravenously to mice at a dose of 50 nmol/kg, and brain was collected after perfusion. The antibody concentrations in brain were measured by ECL. The data show mean+/−SD (n=5-6).

FIG. 20 shows comparison of amyloid-beta 1-40 peptide concentration in brain of mice of which KLH, IL6R/KLH-NEP, IL6R/KLH-TfR-NEP and IL6R/MOG303-TfR-NEP were administered intravenously at a dose of 50 nmol/kg, and brain was collected after perfusion. The amyloid-beta 1-40 peptide concentrations in brain were measured by ECL. The data show mean+/−SD (n=5-6).

FIG. 21(a) shows the plasma concentration-time profiles of the antibodies KLH, BACE1/KLH-TfR and BACE1/MOG303-TfR which were administered intravenously at a dose of 25 mg/kg, and blood was collected. The antibody concentrations in plasma were measured by ECL. The data show mean+/−SD (n=6). FIG. 21(b) shows the brain concentration-time profiles of the antibodies KLH, BACE1/KLH-TfR and BACE1/MOG303-TfR which were administered intravenously at a dose of 25 mg/kg, and brain was collected after perfusion. The antibody concentrations in brain were measured by ELISA. The data show mean+/−SD (n=6).

### DESCRIPTION OF EMBODIMENTS

The definitions and detailed description below are provided to facilitate understanding of the present invention illustrated herein. Especially, these definitions and detailed description serve to interpret the aspects and embodiments of the disclosure.

### Definitions

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art.

The terms “anti-[X] antibody”, “an antibody that binds [X]” and “an antibody that binds to [X]” refer to an antibody that is capable of binding target [X] with sufficient affinity such that the antibody is useful as a diagnostic, preventive and/or therapeutic agent in targeting target X, wherein target [X] is a given target of interest. In one embodiment, the extent of binding of an anti-[X] antibody to an unrelated, non-[X] protein is less than about 10% of the binding of the antibody to [X] as measured, e.g., by a radioimmunoassay (RIA). In certain embodiments, an antibody that binds [X] has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M). In certain embodiments, an anti-[X] antibody binds to an epitope of [X] that is conserved among [X] from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

The term “cytotoxic agent” as used herein refers to a substance that inhibits or prevents a cellular function and/or causes cell death or destruction. Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fe receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-[X] antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa and lambda, based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “pharmaceutical formulation” or “pharmaceutical composition” refer to a preparation which is in such form as to permit the biological activity of an active ingredient contained therein to be effective, and which contains no additional components which are unacceptably toxic to a subject to which the formulation would be administered.

A “pharmaceutically acceptable carrier” or “pharmaceutically acceptable carrier(s) or excipient(s)” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term designating a specific target “[X]”, as used herein, refers to any native target [X] from any vertebrate source, including mammals such as primates (e.g. humans) and rodents (e.g., mice and rats), unless otherwise indicated. The term encompasses “full-length” unprocessed [X] as well as any form of [X] that results from processing in the cell. The term also encompasses naturally occurring variants of [X], e.g., splice variants or allelic variants.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Capable of Binding/Binds to/Binds

In the context of the present invention, the term “binds”, “binds to”, “capable of binding to” and “binding to” are used interchangeably and denote the capacity of a moiety to bind to an antigen in particular under physiological conditions, ie under conditions found in the animal, in particular human body. Particular exemplary methods for determining said capability are described herein below. As used herein, “capability of binding to an antigen” and suchlike may also be designated as “binding affinity for an antigen” and suchlike terms. Furthermore, in case of antibodies, said capability may also be described by using the term “an antibody directed against” or by using the term “an anti antigen X antibody”.

### Specific Binding/Specifically Binds

The terms “specific binding,” “specifically binds,” and the like, refer to the ability of the antigen-binding domain to preferentially bind the respective target relative to other molecules or moieties. In certain embodiments, the antigen-binding domain binds the target with greater affinity, avidity, more readily, and/or with greater duration than it binds another target. Generally, but not necessarily, reference to specific binding means preferential binding where the affinity of the antigen-binding domain to the target is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antigen-binding domain for a different target.

### Compositions and Methods

In one aspect, the invention is based, in part, on the provision antigen-binding molecules with improved blood-brain barrier penetration and retention in brain. Provided are antigen-binding molecules comprising a first antigen-binding domain and a second antigen-binding domain, wherein the first antigen-binding domain specifically binds a first target that facilitates transfer of the antigen-binding molecule into a mammalian brain, and the second antigen-binding domain specifically binds a second target wherein the second target (i) is expressed on the cell membrane of brain cells, or (ii) is a brain ECM protein or a brain ECM polysaccharide, as well as pharmaceutical compositions, and related methods, uses, nucleic acids, vectors, host cells and kits. The antigen-binding molecules of the invention exhibit improved pharmacokinetic properties. The antigen-binding molecules of the invention exhibit increased concentration, exposure and/or retention in brain. The antigen-binding molecules of the invention are useful, e.g., for the diagnosis or treatment of treatment of a brain disorder or disease. In some examples, the brain disorder or diseases is selected from the group consisting of neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heredodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Hallervorden-Spatz syndrome, Lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), psychiatric disorders, cancer (e.g. of the CNS, including brain metastases resulting from cancer elsewhere in the body). In some specific examples, the brain disorder or diseases is selected from the group consisting of Alzheimer's disease, Pompe disease, Frontotemporal dementia (FTD), and Amyotrophic lateral sclerosis (ALS).

First Antigen-Binding Domain that Specifically Binds a First Target

In the present invention, a “first antigen-binding domain” of an “antigen-binding molecule comprising a first antigen-binding domain and a second antigen-binding domain” binds “a first target”, which is referred to as that the first target “facilitates transfer of the antigen-binding molecule into a mammalian brain”.

A target that “facilitates transfer of the antigen-binding molecule into a mammalian brain” is understood in a broadest sense as a target, which results in an increase of the transfer of a compound comprising an antigen-binding domain specifically binding to the target to the brain of a mammal in vivo relative to the compound which does not comprise the antigen-binding domain specifically binding to the target. In certain embodiments, an increase is determined by determining a higher amount or concentration of the compound comprising an antigen-binding domain specifically binding to the target. Generally, but not necessarily, reference to an increase of the transfer means an amount or concentration of the compound comprising an antigen-binding domain in a mammalian brain in vivo that is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the amount or concentration of the compound which does not comprise the antigen-binding domain specifically binding to the target. The transfer into a mammalian brain may be from any tissue, organ or cell type(s) different from brain, and includes for example blood, in particular circulating blood, cerebrospinal fluid, skin, saliva, skin and substructures thereof, eye and substructures thereof and ear and substructures thereof, and the like. In embodiments, the transfer is from circulating blood to brain. The “first target” may have any structure as long as it facilitates transfer into a mammalian brain. For example, the “first target” may be a protein, a carbohydrate, a proteoglycan, a lipid, a nucleic acid or the like. The “first target” may be present in different cell types and organs in a mammal and is not limited to any specific occurrence. In embodiments, the “first target” is present in the vicinity of or on the surface of one or more cells of the blood-brain barrier (BBB), but is not limited thereto. For example, the “first target” includes targets which are located extracellularly, such as proteins, proteoglycans or carbohydrates of the extracellular matrix, and targets which are present on the cell surface of cells, such as a cell surface protein.

The terms “blood-brain barrier” and “BBB” are used herein interchangeably and refer to a selective barrier that separates circulating blood from the brain. The blood-brain barrier comprises a monolayer of endothelial cells bonded by tight junction proteins that form the small cerebral blood vessel lumen. The endothelial cells of the blood-brain barrier are herein referred to as “vascular endothelial cells of the blood-brain barrier” or “microvascular endothelial cells of the blood-brain barrier” or “BMECs”. In addition, astrocytes, in particular, projections from those cells termed astrocytic feet, and pericytes contribute to the structure and function of the blood-brain barrier, and these cells and substructures therefrom are also included as part of the blood-brain barrier. The blood-brain barrier governs entry of all peripherally circulating factors such as water diffusion, some gases and lipid-soluble molecules, and selective transport of other substances, such as glucose, amino acids, and micronutrients that are crucial to neuronal function. Conversely, the blood-brain barrier protects the brain from the passage of toxic substances that may place the central nervous system (CNS) at risk.

The term “brain” is understood as an organ that serves as the center of the nervous system in all vertebrate and most invertebrate animals.

The term “mammalian brain” is understood as brain of a mammal and includes for example, human brain, but is not limited thereto.

In embodiments herein, the mammalian brain is human brain.

The term “brain cell” is understood in the broadest sense to encompass any cell that is present in a mammalian brain. The term “brain cell” includes oligodendrocytes, astrocytes, neurons and microglia but is not limited thereto.

The term “brain-specific cell” is understood in the broadest sense to encompass any cell that is present in a mammalian brain, but is not or essentially not present in other organs or tissues of the mammal. The term “brain-specific cell” includes oligodendrocytes, astrocytes, neurons and microglia but is not limited thereto. A cell that is essentially not present in other organs or tissues of the mammal is a cell for which at least 5-fold, 10-fold, 100-fold or more cells are found in brain than in any other organ or tissue of the mammal.

The term “mammal” includes any mammal, including humans, monkeys, horses, cows, sheep, dogs, cats, cattle, rats and mice, but is not limited thereto. In embodiments, the mammal is a human.

In embodiments, the “first target” is selected from the group consisting of Transferrin receptor (TfR), Basigin (CD147), Glut1, Ldlrad3, CD320, Insulin receptor, insulin-like growth factor 1 receptor (IGF1R), Low density lipoprotein Receptor (LDLR), Low density lipoprotein receptor related protein (LRP), preferably LRP1, Diphtheria toxin Receptor, Glucose receptor, CD98hc, TMEM30A, Leptin receptor (LepR) and heparan sulfate chains branching from proteoglycan (HSPG).

The terms “Transferrin receptor” and “TfR” are used herein interchangeably and refer to a carrier protein for transferrin. The term includes “Transferrin receptor protein 1” or “TfR1” which also known as “Cluster of Differentiation 71” (CD71), and “Transferrin receptor protein 2” or “TfR2”. TfR1 and TfR2 are transmembrane glycoproteins. In an embodiment, TfR is TfR1, preferably human TfR1.

The terms “Basigin” or “CD147” are used herein interchangeably and refer to a transmembrane protein. The UNIPROT reference of human Basigin is P35613. To date, 4 isoforms have been described, designated Isoforms 1 to 4, all of which are encompassed in the term “Basigin”. In an embodiment, Basigin is human Basigin (UNIPROT Accession number is P35613).

The terms “Ldlrad3” and “Low Density Lipoprotein Receptor Class A Domain Containing 3” are used interchangeably herein. The protein belongs to the scavenger receptor superfamily and has been described to be a receptor for Venezuelan equine encephalitis virus. In an embodiment, Ldlrad3 is human Ldlrad3 (UNIPROT Accession number is Q86YD5).

The term “CD320” refers to the transcobalamin receptor. In an embodiment, CD320 is human CD320 (UNIPROT Accession number is Q9NPF0).

The terms “Insulin receptor”, “InsR” and “IR” are used interchangeably herein and refer to a transmembrane receptor that is activated by insulin, IGF-I and IGF-II. In an embodiment, Insulin receptor is human Insulin receptor (UNIPROT Accession number is P06213).

The terms “Low density lipoprotein Receptor”, “LDL-R” and “LDLR” are used interchangeably herein and refer to the Low-density lipoprotein receptor which is a mosaic protein of 839 amino acids (after removal of 21-amino acid signal peptide) that mediates the endocytosis of cholesterol-rich low-density lipoprotein (LDL). It is a cell-surface receptor that recognizes apolipoprotein B100 (ApoB100), which is embedded in the outer phospholipid layer of LDL particles. The receptor also recognizes apolipoprotein E (ApoE) found in chylomicron remnants and very low-density lipoprotein (VLDL) remnants. In an embodiment, LDLR is human LDLR (UNIPROT Accession number is P01130).

The terms “Low density lipoprotein receptor related protein” and “LRP” are used interchangeably herein and refer to members of the family of Low-density lipoprotein receptor-related proteins. The term includes LRP-1, LRP-1b, LRP-2, LRP-5, and LRP-6. In embodiments, LRP is LRP1. In an embodiment, LRP is human LRP1 (UNIPROT Accession number is Q07954).

The terms “Diphtheria toxin Receptor” and “Proheparin-binding EGF-like growth factor” are used interchangeably herein and refer to a growth factor for which the human Diphtheria toxin Receptor protein has UNIPROT Accession number Q99075. In an embodiment, diphtheria toxin Receptor is human diphtheria toxin Receptor (UNIPROT Accession number is Q99075).

The term “Glucose receptor” refers to a family of Glucose Transporter proteins, including, but not limited to Glut1, Glut2 and Glut3. In embodiments, the Glucose Receptor is Glut1. In an embodiment, Glucose receptor is human Glut1 (UNIPROT Accession number is P11166).

The terms “CD98hc” and “CD98 heavy chain” are used interchangeably herein and refer to a cell-surface protein which is also known as “4F2 cell-surface antigen heavy chain” or “SLC3A2”. It is a component of several heterodimeric complexes involved in amino acid transport, including the Glucose Receptor. In an embodiment, CD98hc is human CD98hc (UNIPROT Accession number is P08195).

The terms “TMEM30A” and “Cell cycle control protein 50A” are used interchangeably, and refers to a protein for which the human TMEM30A has UNIPROT Accession number Q9NV96. In an embodiment, TMEM30A is human TMEM30A (UNIPROT Accession number is Q9NV96).

The terms “Leptin receptor”, “Lep-R” and “LepR” are used interchangeably herein and refer to a type I cytokine receptor, a protein that in humans is encoded by the LEPR gene. Lep-R functions as a receptor for the fat cell-specific hormone leptin. In an embodiment, Leptin receptor is human Leptin receptor (UNIPROT Accession number is P48357).

The term “heparan sulfate chains branching from proteoglycan (HSPG)” refers to the heparan sulfate chains which are covalently attached to glycoproteins. Heparan sulfate chains are a type of glycosaminoglycan (GAG). The term includes membrane HSPGs, such as syndecans and glycosylphosphatidylinositol-anchored proteoglycans (glypicans), the secreted extracellular matrix HSPGs (agrin, perlecan, type XVIII collagen), and the secretory vesicle proteoglycan, serglycin. In an embodiment, HSPG is human HSPG1 (UNIPROT Accession number is P34741).

“A first antigen-binding domain” of the molecule in the present invention may have any structure as long as it specifically binds “a first antigen” as described above. The structure of “a first antigen-binding domain” may include but is not limited to, a polypeptide or a portion thereof, or a small or medium chemical compound or a portion thereof, or a polynucleotide or a portion thereof. The polypeptide or a portion thereof includes but is not limited to a cell membrane protein expressed on a cell (e.g., an immune cell such as a dendritic cell) or a portion thereof (e.g., an extracellular domain, any unique domain thereof); an antibody (including but not limited to a human antibody, a chimeric, antibody, a humanized antibody, and VHH antibody) or an antigen-binding domain (also referred as a portion, a part or a fragment of an antibody). The antigen-binding domain of an antibody includes but is not limited to an antibody heavy chain variable (VH) region, an antibody light chain variable (VL) region (preferably a combination of an antibody heavy chain variable (VH) region and an antibody light chain variable (VL) region), a single-domain antibody (sdAb), a single-chain Fv (scFv), a single-chain antibody, a Fv, a single-chain Fv2 (scFv2), a Fab, and F (ab′)2.

The polypeptide or a portion thereof may also be an antigen binding polypeptides such as a module called A domain of Avimer, which has approximately 35 amino acids contained in an in vivo cell membrane protein (WO2004/044011 and WO2005/040229), adnectin having a 10Fn3 domain serving as a protein binding domain, which is derived from a glycoprotein fibronectin expressed on cell membranes (WO2002/032925), Affibody having an IgG binding domain scaffold constituting a three-helix bundle composed of 58 amino acids of protein A (WO1995/001937), DARPins (designed ankyrin repeat proteins) which are molecular surface-exposed regions of ankyrin repeats (AR) each having a 33-amino acid residue structure folded into a subunit of a turn, two antiparallel helices, and a loop (WO2002/020565), anticalin having four loop regions connecting eight antiparallel strands bent toward the central axis in one end of a barrel structure highly conserved in lipocalin molecules such as neutrophil gelatinase-associated lipocalin (NGAL) (WO2003/029462), and a depressed region in the internal parallel sheet structure of a horseshoe-shaped fold composed of repeated leucine-rich-repeat (LRR) modules of an immunoglobulin structure-free variable lymphocyte receptor (VLR) as seen in the acquired immune systems of jawless vertebrates such as lamprey or hagfish (WO2008/016854).

One embodiment of a polypeptide or a portion thereof which belongs to “a first antigen-binding domain” as described above in the present invention includes but is not limited to a cell membrane protein expressed on a cell (e.g., a receptor) or a portion thereof (e.g., an extracellular domain, any unique domain thereof).

The polypeptide or a portion thereof includes but is not limited to an antibody (including but not limited to a human antibody, a chimeric, antibody, a humanized antibody, and VHH antibody) or an antigen-binding domain (also referred as a portion, a part or a fragment of an antibody). The antigen-binding domain of an antibody includes but is not limited to an antibody heavy chain variable (VH) region, an antibody light chain variable (VL) region (preferably a combination of an antibody heavy chain variable (VH) region and an antibody light chain variable (VL) region), a single-domain antibody (sdAb), a single-chain Fv (scFv), a single-chain antibody, a Fv, a single-chain Fv2 (scFv2), a Fab, and F (ab′)2.

In certain embodiments herein, mammalian brain is human brain. Accordingly, in certain embodiments herein, the “first target that facilitates transfer of the antigen-binding molecule into a mammalian brain” is a first target that facilitates transfer of the antigen-binding molecule into a human brain.

Second Antigen-Binding Domain that Specifically Binds a Second Target

In the present invention, a “second antigen-binding domain” of an “antigen-binding molecule comprising a first antigen-binding domain and a second antigen-binding domain” binds “a second target”, which is referred to as that the second target “is expressed on the cell membrane of brain cells, or is a brain ECM protein or a brain ECM polysaccharide”.

In embodiments, the second target is different from the first target, as described above.

A target that “is expressed on the cell membrane of brain cells” is understood in a broadest sense as a target which is expressed on the cell membrane of at least one cell type of brain cells. For example, the target may be expressed on the cell membrane of 2, 3, 4, 5 or more different brain cells, such as, for example, on the cell membrane of neurons and oligodendrocytes. The term “expressed on the cell membrane of brain cells” includes molecules, such as proteins, which comprise at least one transmembrane protein and at least one extracellular protein associated with the transmembrane protein, as well as a secreted polypeptides and proteoglycans and polypeptides which are inserted into a cell membrane, such as by a lipid anchor, or are otherwise associated with the cell membrane of brain cells, such as by non-covalent binding, such as by binding of a ligand to a receptor.

The “second target” may have any structure as long as it is expressed on the cell membrane of brain cells. For example, the “second target” may be a protein, a carbohydrate, a proteoglycan, a lipid, a nucleic acid or the like. The “second target” may be present in different cell types and organs in a mammal and is not limited to any specific occurrence. For example, in embodiments, the “second target” is selected from the group consisting of Myelin Oligodendrocyte glycoprotein (MOG), Neuroglycan C/Chondroitin sulfate proteoglycan 5 (CSPG5); IGSF4B/SynCAM3/Cell Adhesion Molecule 3 (CADM3); CNPase (2′,3′-cyclic nucleotide 3′-phosphodiesterase); Myelin-associated Glycoprotein (MAG), Myelin Basic Protein (MBP), EAAT1 (Solute Carrier Family 1 member 3), EAAT2 (Solute Carrier Family 1 member 2), MAP2 (Microtubule-associated protein2), NEFL (Neurofilament light polypeptide), NEFM (Neurofilament medium polypeptide), NSE (Gamma-enolase), CD68 (Macrosialin), Allograft inflammatory factor 1 (IBA1 or AIF1), Purinergic receptor (P2RY12), Interleukin 1 receptor accessory protein like 1 (ILIRAPL1), Glutamate ionotropic receptor NMDA type subunit 2B (GRIN2B), Calcium voltage-gated channel auxiliary subunit gamma 8 (CACNG8), CD11b (Integrin subunit alpha M), SLC6A2 (Sodium-dependent noradrenaline transporter), DPP6 (Dipeptidyl peptidase like 6), SLC18A3 (Vesicular acetylcholine transporter), Sodium/potassium-transporting ATPase subunit alpha-2, Broad substrate specificity ATP-binding cassette transporter ABCG2, Solute carrier family 12 member 9, Electrogenic sodium bicarbonate cotransporter 1, Excitatory amino acid transporter 2, Chondroitin sulfate proteoglycan 4, Immunoglobulin superfamily DCC subclass member 4, Vang-like protein 2, Neural cell adhesion molecule 1 (N-CAM-1), Low-density lipoprotein receptor-related protein 4 (LRP-4), Phosphoprotein associated with glycosphingolipid-enriched microdomains 1 (Csk-binding protein), Plasma membrane calcium-transporting ATPase 1, Prominin-1, Somatostatin receptor type 1, Carnitine O-palmitoyltransferase 1 brain isoform (CPT1-B), Epidermal growth factor receptor, Protein MAL2, Syntaxin-1A, Sodium/calcium exchanger 1, Lysophosphatidylcholine acyltransferase 1 (LPC acyltransferase 1), Calsyntenin-3 (Alcadein-beta), Pituitary adenylate cyclase-activating polypeptide type I receptor (PACAP type I receptor), Neutral cholesterol ester hydrolase 1 (NCEH), CD166 antigen (Activated leukocyte cell adhesion molecule), Inactive tyrosine-protein kinase 7, Claudin-11, Ectonucleotide phosphatase (ENPP6), Tetraspanin-2 (Tspan-2), Myelin proteolipid protein (PLP), Glycolipid transfer protein (GLTP).

A target that “is a brain ECM protein or a brain polysaccharide” is understood in the broadest sense as a protein or polysaccharide that is part of the brain extracellular matrix (ECM). The “brain ECM” is understood as “extracellular matrix” or “ECM” of the brain and is a macromolecular network primarily composed of polysaccharide glycosaminoglycans (e.g., hyaluronan), proteoglycans (e.g., neurocan, brevican, versican and aggrecan), glycoproteins (e.g., tenascin-R), and low levels of fibrous proteins (e.g. collagen, fibronectin, and vitronectin). Examples of brain ECM proteins, include are but not limited to, Versican core protein (Chondroitin sulfate proteoglycan 2 or CSPG2), Tropoelastin (Elastin), Collagen alpha-2(IV) chain (Canstatin), Proteoglycan link protein 1 (Hyaluronan and proteoglycan link protein 1), Tenascin-R (TN-R), Proteoglycan link protein 2 (Hyaluronan and proteoglycan link protein 2), Collagen alpha-1(I) chain, Neurofilament-3 (NEF3), Immunoglobulin superfamily member 8 (IgSF8), Laminin subunit gamma-1 (LAMC1), Collagen alpha-1(VI) chain (Col6a1), and Collagen alpha-3(VI) chain (Col6a3). Examples of a brain ECM polysaccharide, includes but is not limited to hyaluronan.

These targets and their sequence and/or structure are well-known in the art.

In some embodiments, the “second target” is predominantly expressed on brain-specific cell(s).

A “target that is predominantly expressed on brain-specific cell(s)” is understood as a target for which expression on the cell membrane of brain-specific cells is at least 2-fold, 5-fold, 10-fold, 50-fold or 100-fold higher than in non-brain specific cells. A “target that is predominantly expressed on brain-specific cell(s)” also includes a target which is expressed on the cell membrane of less than 10, less than 5, less than 4, less than 3 or less than 2 other tissues or organs different from brain. In one embodiment, the target is only expressed on the cell membrane of brain-specific cell(s).

In further embodiments, the second target is a molecule that is broadly expressed in brain tissue(s).

A target that “is a molecule that is broadly expressed in brain tissue(s)” is understood as target which is expressed in more than one brain tissue(s) selected from the group consisting of cerebral cortex, hippocampal formation, amygdala, basal ganglia, thalamus, hypothalamus, midbrain, cerebellum, pons, medulla oblongata and spinal cord. In embodiments, a target that “is a molecule that is broadly expressed in brain tissue(s)” is a target which is expressed in more than two, or more than three brain tissues selected from the group consisting of cerebral cortex, hippocampal formation, amygdala, basal ganglia, thalamus, hypothalamus, midbrain, cerebellum, pons, medulla oblongata, spinal cord. For example, the second target “that is broadly expressed in brain tissue(s)” may be a molecule selected from the group consisting of Interleukin 1 receptor accessory protein like 1 (ILIRAPL1), Glutamate ionotropic receptor NMDA type subunit 2B (GRIN2B), and Calcium voltage-gated channel auxiliary subunit gamma 8 (CACNG8). In one embodiment, the second target is only expressed in brain tissue(s).

The terms “Myelin Oligodendrocyte glycoprotein” and “MOG” are used interchangeably herein and refer to a member of the immunoglobulin (Ig) superfamily which is a myelin protein solely expressed at the outermost surface of myelin sheaths and oligodendrocyte membranes. In an embodiment, MOG is human MOG (UNIPROT Accession number is Q16653).

The terms “Neuroglycan C”, “Chondroitin sulfate proteoglycan 5” and “CSPG5” are used interchangeably herein and refer to a brain-specific chondroitin sulfate proteoglycan. Neuroglycan C is a 120-150 kDa type I transmembrane glycoprotein and member of the neuregulin family of proteins. Depending on its expression, Neuroglycan C may be a glycoprotein of 120 kDa, or a chondroitin sulfate (CS) proteoglycan of 150 kDa, and both variants are included herein. In an embodiment, Neuroglycan C is human Neuroglycan C (UNIPROT Accession number is 095196).

The terms “IGSF4B”, “Cell adhesion molecule 3” and “SynCAM3 (CADM3)” are used interchangeably herein and refer to a protein involved in the cell-cell adhesion. In an embodiment, IGSF4B is human IGSF4B (UNIPROT Accession number is Q8N126).

The terms “CNPase” and “2′,3′-cyclic nucleotide 3′-phosphodiesterase” are used interchangeably herein and refer to an enzyme that in humans is encoded by the CNP gene. CNPase is a myelin-associated enzyme that makes up 4% of total CNS myelin protein. In an embodiment, CNPase is human CNPase (UNIPROT Accession number is P09543).

The terms “Myelin-associated Glycoprotein” and “MAG” are used interchangeably herein and refer to a type 1 transmembrane protein glycoprotein localized in periaxonal Schwann cell and oligodendrocyte membranes, where it plays a role in glial-axonal interactions. MAG is a member of the SIGLEC family of proteins and is a functional ligand of the NOGO-66 receptor, NgR. In an embodiment, MAG is human MAG (UNIPROT Accession number is P20916).

The terms “Myelin Basic Protein” and “MBP” are used interchangeably herein and refer to the Myeloin Basic Protein which is the major constituent of the myelin sheath of oligodendrocytes and Schwann cells. The human protein has UNIPROT Accession number P02686. In an embodiment, MBP is human MBP (UNIPROT Accession number is P02686).

The terms “EAAT1” and “Solute Carrier F” are used interchangeably herein and refer to protein Excitatory amino acid transporter 1. EEAT1 is a protein that, in humans, is encoded by the SLC1A3 gene. EAAT1 is also designated the GLutamate ASpartate Transporter 1 (GLAST-1). In an embodiment, EAAT1 is human EAAT1 (UNIPROT Accession number is P43003).

The terms “EAAT2” and “Solute Carrier Family 1 member 2” are used interchangeably herein and refer to protein Excitatory amino acid transporter 2. EEAT2 is a protein that, in humans, is encoded by the SLC1A2 gene. EAAT2 is also designated the glutamate transporter 1 (GLT-1). In an embodiment, EAAT2 is human EAAT2 (UNIPROT Accession number is P43004).

The terms “MAP2” and “Microtubule-associated protein2” are used interchangeably herein and refer to a protein that belongs to the microtubule-associated protein family. In humans the protein is encoded by the MAP2 gene. In an embodiment, MAP2 is human MAP2 (UNIPROT Accession number is P11137).

The terms “NEFL” and “Neurofilament light polypeptide” are used interchangeably herein and refer to neurofilament light chain. NEFL a neurofilament protein that in humans is encoded by the NEFL gene. Neurofilament light chain is a biomarker that can be measured with immunoassays in cerebrospinal fluid and plasma and reflects axonal damage in a wide variety of neurological disorders. In an embodiment, NEFL is human NEFL (UNIPROT Accession number is P07196).

The terms “NEM” and “Neurofilament medium polypeptide” are used interchangeably herein and refer to Neurofilament medium polypeptide which is a protein that in humans is encoded by the NEFM gene. Neurofilaments are type IV intermediate filament heteropolymers composed of light (NEFL), medium (this protein), and heavy (NEFH) chains. In an embodiment, NEFM is human NEFM (UNIPROT Accession number is P07197).

The terms “NSE” and “Gamma-enolase” are used interchangeably herein and refer to enolase 2 (ENO2) or neuron specific enolase (NSE), which is an enzyme that in humans is encoded by the ENO2 gene. Gamma-enolase is a phosphopyruvate hydratase. Gamma-enolase is one of the three enolase isoenzymes found in mammals. Isoenzyme Gamma-enolase, which is a homodimer, is found in mature neurons and cells of neuronal origin. In an embodiment, NSE is human NSE (UNIPROT Accession number is P09104).

The terms “CD68” and “Macrosialin” are used interchangeably herein and refer to antigen termed “Cluster of Differentiation 68”. The human protein has UNIPROT Accession number P34810. Human CD68 is a transmembrane glycoprotein which is heavily glycosylated in its extracellular domain. In an embodiment, CD68 is human CD68 (UNIPROT Accession number is P34810).

The terms “Allograft inflammatory factor 1” and “IBA1” and “AIF1” are used interchangeably herein and refer to Ionized calcium-binding adapter molecule 1. AIF1 is a protein that in humans is encoded by the AIF1 gene. In an embodiment, IBA1 is human IBA1 (UNIPROT Accession number is P55008).

The terms “Purinergic receptor” and “P2RY12” are used interchangeably herein and refer to a chemoreceptor for adenosine diphosphate (ADP) that belongs to the Gi class of a group of G protein-coupled (GPCR) purinergic receptor. The human protein has UNIPROT Accession number Q9H244. In an embodiment, P2RY12 is human P2RY12 (UNIPROT Accession number is Q9H244).

The terms “Interleukin 1 receptor accessory protein like 1” and “ILIRAPL1” are used interchangeably herein and refer to X-linked interleukin-1 receptor accessory protein-like 1. This is a protein that in humans is encoded by the ILIRAPL1 gene. In an embodiment, ILIRAPL1 is human ILIRAPL1 (UNIPROT Accession number is Q9NZN1).

The terms “Glutamate ionotropic receptor NMDA type subunit 2B” and “GRIN2B” are used interchangeably herein and refer to N-methyl D-aspartate receptor subtype 2B (NMDAR2B or NR2B). This is a protein that in humans is encoded by the GRIN2B gene. The human protein has UNIPROT Accession number Q13224. In an embodiment, GRIN2B is human GRIN2B (UNIPROT Accession number is Q13224).

The terms “Calcium voltage-gated channel auxiliary subunit gamma 8” and “CACNG8” are used interchangeably herein and refer to a protein for which the human CACGN8 has UNIPROT Accession number Q8WXS5. In an embodiment, CACNG8 is human CACNG8 (UNIPROT Accession number is Q8WXS5).

The terms “CD11b” and “Integrin subunit alpha M” are used interchangeably herein and refer to a protein for which the human CD11b has UNIPROT Accession number P11215. In an embodiment, CD11b is human CD11b (UNIPROT Accession number is P11215).

The terms “SLC6A2” and “Sodium-dependent noradrenaline transporter” are used interchangeably herein and refer to a protein for which the human SLC6A2 has UNIPROT Accession number P23975. In an embodiment, SLC6A2 is human SLC6A2 (UNIPROT Accession number is P23975).

The terms “DPP6” and “Dipeptidyl peptidase like 6” are used interchangeably herein and refer to a protein for which the human DPP6 has UNIPROT Accession number P42658. In an embodiment, DPP6 is human DPP6 (UNIPROT Accession number is P42658).

The terms “SLC18A3” and “Vesicular acetylcholine transporter” are used interchangeably herein and refer to a protein for which the human SLC18A3 has UNIPROT Accession number Q16572. In an embodiment, SLC18A3 is human SLC18A3 (UNIPROT Accession number is Q16572.

The term “Sodium/potassium-transporting ATPase subunit alpha-2” refers to a protein for which the human Sodium/potassium-transporting ATPase subunit alpha-2 has UNIPROT Accession number P50993. In an embodiment, Sodium/potassium-transporting ATPase subunit alpha-2 is human Sodium/potassium-transporting ATPase subunit alpha-2 (UNIPROT Accession number is P50993.

The term “Broad substrate specificity ATP-binding cassette transporter ABCG2” refers to a protein for which the human broad substrate specificity ATP-binding cassette transporter ABCG2 has UNIPROT Accession number Q9UNQ0. In an embodiment, broad substrate specificity ATP-binding cassette transporter ABCG2 is human broad substrate specificity ATP-binding cassette transporter ABCG2 (UNIPROT Accession number is Q9UNQ0.

The term “Solute carrier family 12 member 9” refers to a protein for which the human Solute carrier family 12 member 9 has UNIPROT Accession number Q9BXP2. In an embodiment, solute carrier family 12 member 9 is human solute carrier family 12 member 9 (UNIPROT Accession number is Q9BXP2.

The term “Electrogenic sodium bicarbonate cotransporter 1” refers to a protein for which the human Electrogenic sodium bicarbonate cotransporter 1 has UNIPROT Accession number Q9Y6R1. In an embodiment, Electrogenic sodium bicarbonate cotransporter 1 is human Electrogenic sodium bicarbonate cotransporter 1 (UNIPROT Accession number is Q9Y6R1.

The term “Excitatory amino acid transporter 2” refers to a protein for which the human Excitatory amino acid transporter 2 has UNIPROT Accession number P43004. In an embodiment, Excitatory amino acid transporter 2 is human Excitatory amino acid transporter 2 (UNIPROT Accession number is P43004.

The term “Chondroitin sulfate proteoglycan 4” refers to a protein for which the human Chondroitin sulfate proteoglycan 4 has UNIPROT Accession number Q6UVK1. In an embodiment, Chondroitin sulfate proteoglycan 4 is human Chondroitin sulfate proteoglycan 4 (UNIPROT Accession number is Q6UVK1.

The term “Excitatory amino acid transporter 2” refers to a protein for which the human Excitatory amino acid transporter 2 has UNIPROT Accession number P43004. In an embodiment, Excitatory amino acid transporter 2 is human Excitatory amino acid transporter 2 (UNIPROT Accession number is P43004.

The term “Immunoglobulin superfamily DCC subclass member 4” refers to a protein for which the human Immunoglobulin superfamily DCC subclass member 4 has UNIPROT Accession number Q8TDY8. In an embodiment, Immunoglobulin superfamily DCC subclass member 4 is human Immunoglobulin superfamily DCC subclass member 4 (UNIPROT Accession number is Q8TDY8.

The term “Vang-like protein 2” refers to a protein for which the human Vang-like protein 2 has UNIPROT Accession number Q9ULK5. In an embodiment, Vang-like protein 2 is human Vang-like protein 2 (UNIPROT Accession number is Q9ULK5.

The terms “Neural cell adhesion molecule 1” and “N-CAM-1” are used interchangeably herein and refer to a protein for which the human N-CAM-1 has UNIPROT Accession number P13591. In an embodiment, N-CAM-1 is human N-CAM-1 (UNIPROT Accession number is P13591.

The terms “Low-density lipoprotein receptor-related protein 4” and “LRP-4” are used interchangeably herein and refer to a protein for which the human LRP-4 has UNIPROT Accession number 075096. In an embodiment, LRP-4 is human LRP-4 (UNIPROT Accession number is 075096.

The terms “Phosphoprotein associated with glycosphingolipid-enriched microdomains 1” and “Csk-binding protein” are used interchangeably herein and refer to a protein for which the human Phosphoprotein associated with glycosphingolipid-enriched microdomains 1 has UNIPROT Accession number Q9NWQ8. In an embodiment, Phosphoprotein associated with glycosphingolipid-enriched microdomains 1 is human Phosphoprotein associated with glycosphingolipid-enriched microdomains 1 (UNIPROT Accession number is Q9NWQ8.

The term “Plasma membrane calcium-transporting ATPase 1” refers to a protein for which the human Plasma membrane calcium-transporting ATPase 1 has UNIPROT Accession number P20020. In an embodiment, Plasma membrane calcium-transporting ATPase 1 is human Plasma membrane calcium-transporting ATPase 1 (UNIPROT Accession number is P20020.

The term “Prominin-1” refers to a protein for which the human Prominin-1 has UNIPROT Accession number 043490. In an embodiment, Prominin-1 is human Prominin-1 (UNIPROT Accession number is 043490.

The term “Somatostatin receptor type 1” refers to a protein for which the human Somatostatin receptor type 1 has UNIPROT Accession number P30872. In an embodiment, Somatostatin receptor type 1 is human Somatostatin receptor type 1 (UNIPROT Accession number is P30872.

The terms “Carnitine 0-palmitoyltransferase 1, brain isoform” and “CPT1-B” are used interchangeably herein and refer to a protein for which the human Carnitine 0-palmitoyltransferase 1, brain isoform has UNIPROT Accession number Q8TCG5. In an embodiment, Carnitine 0-palmitoyltransferase 1, brain isoform is human Carnitine 0-palmitoyltransferase 1, brain isoform (UNIPROT Accession number is Q8TCG5.

The term “Epidermal growth factor receptor” refers to a protein for which the human Epidermal growth factor receptor has UNIPROT Accession number P00533. In an embodiment, Epidermal growth factor receptor is human Epidermal growth factor receptor (UNIPROT Accession number is P00533.

The term “Protein MAL2” refers to a protein for which the human Protein MAL2 has UNIPROT Accession number Q969L2. In an embodiment, Protein MAL2 is human Protein MAL2 (UNIPROT Accession number is Q969L2.

The term “Syntaxin-1A” refers to a protein for which the human Syntaxin-1A has UNIPROT Accession number Q16623. In an embodiment, Syntaxin-1A is human Syntaxin-1A (UNIPROT Accession number is Q16623.

The term “Sodium/calcium exchanger 1” refers to a protein for which the human Sodium/calcium exchanger 1 has UNIPROT Accession number P32418. In an embodiment, Sodium/calcium exchanger 1 is human Sodium/calcium exchanger 1 (UNIPROT Accession number is P32418.

The terms “Lysophosphatidylcholine acyltransferase 1” and “LPC acyltransferase 1” are used interchangeably herein and refer to a protein for which the human LPC acyltransferase 1 has UNIPROT Accession number Q8NF37. In an embodiment, LPC acyltransferase 1 is human LPC acyltransferase 1 (UNIPROT Accession number is Q8NF37.

The terms “Calsyntenin-3” and “Alcadein-beta” are used interchangeably herein and refer to a protein for which the human Calsyntenin-3 has UNIPROT Accession number Q9BQT9. In an embodiment, Calsyntenin-3 is human Calsyntenin-3 (UNIPROT Accession number is Q9BQT9.

The terms “Pituitary adenylate cyclase-activating polypeptide type I receptor” and “PACAP type I receptor” are used interchangeably herein and refer to a protein for which the human PACAP type I receptor has UNIPROT Accession number P41586. In an embodiment, PACAP type I receptor is human PACAP type I receptor (UNIPROT Accession number is P41586.

The terms “Neutral cholesterol ester hydrolase 1” and “NCEH” are used interchangeably herein and refer to a protein for which the human NCEH has UNIPROT Accession number Q6PIU2. In an embodiment, NCEH is human NCEH (UNIPROT Accession number is Q6PIU2.

The terms “CD166” and “Activated leukocyte cell adhesion molecule” are used interchangeably herein and refer to a protein for which the human CD166 has UNIPROT Accession number Q13740. In an embodiment, CD166 is human CD166 (UNIPROT Accession number is Q13740.

The term “Inactive tyrosine-protein kinase 7” refers to a protein for which the human Inactive tyrosine-protein kinase 7 has UNIPROT Accession number Q13308. In an embodiment, Inactive tyrosine-protein kinase 7 is human Inactive tyrosine-protein kinase 7 (UNIPROT Accession number is Q13308.

The term “Claudin-11” refers to a protein for which the human Claudin-11 has UNIPROT Accession number 075508. In an embodiment, Claudin-11 is human Claudin-11 (UNIPROT Accession number is 075508.

The terms “Ectonucleotide phosphatase” and “ENPP6” are used interchangeably herein and refer to a protein for which the human ENPP6 has UNIPROT Accession number Q6UWR7. In an embodiment, ENPP6 is human ENPP6 (UNIPROT Accession number is Q6UWR7.

The terms “Tetraspanin-2” and “Tspan-2” are used interchangeably herein and refer to a protein for which the human Tetraspanin-2 has UNIPROT Accession number 060636. In an embodiment, Tetraspanin-2 is human Tetraspanin-2 (UNIPROT Accession number is 060636.

The terms “Myelin proteolipid protein” and “PLP” are used interchangeably herein and refer to a protein for which the human Myelin proteolipid protein has UNIPROT Accession number P60201. In an embodiment, Myelin proteolipid protein is human Myelin proteolipid protein (UNIPROT Accession number is P60201.

The terms “Glycolipid transfer protein” and “GLTP” are used interchangeably herein and refer to a protein for which the human GLTP has UNIPROT Accession number Q9NZD2. In an embodiment, GLTP is human GLTP (UNIPROT Accession number is Q9NZD2.

The terms “Versican core protein”, “Chondroitin sulfate proteoglycan 2” and “CSPG2” are used interchangeably herein and refer to a protein for which the human CSPG2 has UNIPROT Accession number P13611. In an embodiment, CSPG2 is human CSPG2 (UNIPROT Accession number is P13611.

The terms “Tropoelastin” and “Elastin” are used interchangeably herein and refer to a protein for which the human Tropoelastin has UNIPROT Accession number P15502. In an embodiment, Tropoelastin is human Tropoelastin (UNIPROT Accession number is P15502.

The terms “Collagen alpha-2(IV) chain” and “Canstatin” are used interchangeably herein and refer to a protein for which the human Collagen alpha-2(IV) chain has UNIPROT Accession number P08572. In an embodiment, Collagen alpha-2(IV) chain is human Collagen alpha-2(IV) chain (UNIPROT Accession number is P08572.

The terms “Proteoglycan link protein 1” and “Hyaluronan and proteoglycan link protein 1” are used interchangeably herein and refer to a protein for which the human Proteoglycan link protein 1 has UNIPROT Accession number P10915. In an embodiment, Proteoglycan link protein 1 is human Proteoglycan link protein 1 (UNIPROT Accession number is P10915.

The terms “Tenascin-R” and “TN-R” are used interchangeably herein and refer to a protein for which the human Tenascin-R has UNIPROT Accession number Q92752. In an embodiment, Tenascin-R is human Tenascin-R (UNIPROT Accession number is Q92752.

The terms “Proteoglycan link protein 2” and “Hyaluronan and proteoglycan link protein 2” are used interchangeably herein and refer to a protein for which the human Proteoglycan link protein 2 has UNIPROT Accession number Q9GZV7. In an embodiment, Proteoglycan link protein 2 is human Proteoglycan link protein 2 (UNIPROT Accession number is Q9GZV7.

The term “Collagen alpha-1(I) chain” refers to a protein for which the human Collagen alpha-1(I) chain has UNIPROT Accession number P02452. In an embodiment, Collagen alpha-1(I) chain is human Collagen alpha-1(I) chain (UNIPROT Accession number is P02452.

The terms “Neurofilament-3” and “NEF3” are used interchangeably herein and refer to a protein for which the human Neurofilament-3 has UNIPROT Accession number Q9UK51. In an embodiment, Neurofilament-3 is human Neurofilament-3 (UNIPROT Accession number is Q9UK51.

The terms “Immunoglobulin superfamily member 8” and “IgSF8” are used interchangeably herein and refer to a protein for which the human IgSF8 has UNIPROT Accession number Q969P0. In an embodiment, IgSF8 is human IgSF8 (UNIPROT Accession number is Q969P0.

The terms “Laminin subunit gamma-1” and “LAMC1” are used interchangeably herein and refer to a protein for which the human Laminin subunit gamma-1 has UNIPROT Accession number P11047. In an embodiment, Laminin subunit gamma-1 is human Laminin subunit gamma-1 (UNIPROT Accession number is P11047.

The terms “Collagen alpha-1(VI) chain” and “Col6a1” are used interchangeably herein and refer to a protein for which the human Collagen alpha-1(VI) chain has UNIPROT Accession number P12109. In an embodiment, Collagen alpha-1(VI) chain is human Collagen alpha-1(VI) chain (UNIPROT Accession number is P12109.

The terms “Collagen alpha-3(VI) chain” and “Col6a3” are used interchangeably herein and refer to a protein for which the human Collagen alpha-3(VI) chain has UNIPROT Accession number P12111. In an embodiment, Collagen alpha-3(VI) chain is human Collagen alpha-3(VI) chain (UNIPROT Accession number is P12111.

In certain embodiments herein, the second target is not a target selected from the group consisting of beta-secretase 1 (BACE1), Abeta, epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, TRK A, TRK B, TRK C, an alpha synuclein, a beta synuclein, a gamma synuclein, vascular endothelial growth factor (VEGF), neuropilin, a Semaphorin, Semaphorin 3A, Semaphorin 4A, Semaphorin 6A, myelin basic protein (MBP), MOG, PLP, MAG, aquaporin 4, glutamate receptor, and EpCAM.

The terms “beta-secretase 1” and “BACE1” are used interchangeably herein and refer to a protein which is also known as “beta-site amyloid precursor protein cleaving enzyme 1”. The human protein has UNIPROT Accession number P56817.

The terms “Abeta” and “Amyloid beta” are used interchangeably herein and refer to peptides of 36-43 amino acids that are the main component of the amyloid plaques found in the brains of subjects with Alzheimer's disease.

The terms “epidermal growth factor receptor” and “EGFR” are used interchangeably herein. The human protein has UNIPROT Accession number P00533.

The terms “human epidermal growth factor receptor 2” and “HER2” are used interchangeably herein and refer to a protein which is also known as “Receptor tyrosine-protein kinase erbB-2”. The human protein has UNIPROT Accession number P04626.

The terms “tau” and “Microtubule-associated protein tau” are used interchangeably herein. The human protein has UNIPROT Accession number P10636.

The terms “apolipoprotein E” and “ApoE” are used interchangeably herein. Various variants and isoforms of ApoE have been described which are included in the term “ApoE”, such as variant E4.

The terms “alpha-synuclein” and “alpha synuclein” are used interchangeably herein and refer to a protein that, in humans, is encoded by the SNCA gene. Alpha-synuclein is a neuronal protein that regulates synaptic vesicle trafficking and subsequent neurotransmitter release.

The term “CD20” refer to a protein which is also known as “B-lymphocyte antigen CD20”. In humans, CD20 is encoded by the MS4A1 gene.

The terms “huntingtin” and “Htt” are used interchangeably herein and refer to a protein encoded by the HTT gene in humans, which is also known as the IT15 (“interesting transcript 15”) gene.

The terms “prion protein” and “PrP” are used interchangeably herein and refer to a protein which is encoded by the PRNP gene in humans. Various isoforms of the protein exist which are included in the term “PrP”, such as the PrPC form, the protease-resistant form designated PrPRes such as the disease-causing PrPSc(scrapie) and the isoform located in mitochondria.

The terms “Leucine-rich repeat kinase 2” and “LRRK2” are used interchangeably herein and refer to a protein encoded by the LRKK2 gene in humans. The protein is also known as dardarin or PARK8.

The term “parkin” refers to a protein that, in humans, is encoded by the PSEN-1 gene. Parkin is a 465-amino acid residue E3 ubiquitin ligase.

The term “Presenilin-1” refers to a protein that, in humans, is encoded by the PSEN-1 gene. Presenilin-1 is one of the four core proteins in the gamma secretase complex, which is considered to play an important role in generation of amyloid beta (A beta) from amyloid precursor protein (APP).

The term “Presenilin-2” refers to a protein that, in humans, is encoded by the PSEN2 gene. It has been described that it is a probable catalytic subunit of the gamma-secretase complex.

The term “gamma secretase” refers to a multi-subunit protease complex which consists of four individual proteins, PSEN1 (presenilin-1), nicastrin, APH-1 (anterior pharynx-defective 1), and PEN-2 (presenilin enhancer 2). The complex itself is an integral membrane protein that cleaves single-pass transmembrane proteins at residues within the transmembrane domain. The most well-known substrate of gamma secretase is amyloid precursor protein.

The terms “Death receptor 6” and “DR6” are used interchangeably herein and refer to a protein also known as “tumor necrosis factor receptor superfamily member 21”.

The terms “Amyloid precursor protein” and “APP” are used interchangeably herein and refer to the precursor protein of Abeta.

The terms “p75 neurotrophin receptor” and “p75NTR” are used interchangeably herein and refer to a protein encoded by the NGFR gene in humans.

The terms “Caspase 6” and “Caspase-6” are used interchangeably herein and refer to a protein encoded by the CASP6 gene in humans.

The term “Trk A” refers to Tropomyosin receptor kinase A. TrkA is a protein encoded by the NTRK1 gene.

The term “Trk B” refers to Tropomyosin receptor kinase B. TrkB is a protein encoded by the NTRK2 gene.

The term “Trk C” refers to Tropomyosin receptor kinase C. TrkC is a protein encoded by the NTRK3 gene.

The terms “beta-synuclein” and “beta synuclein” are used interchangeably herein and refer to a protein that, in humans, is encoded by the SNCB gene.

The terms “gamma-synuclein” and “gamma synuclein” are used interchangeably herein and refer to a protein that, in humans, is encoded by the SNCG gene.

The terms “Vascular endothelial growth factor” and “VEGF” are used interchangeably herein and refer to a protein that, in humans, has UNIPROT Accession number Q9UNS8.

The term “neuropilin” refers to a protein receptor, for which two forms are described, NRP-1 and NRP-2, both of which are included in the term “neuropilin”. In humans, NRP-1 has UNIPROT Accession number 014786. In humans, NRP-2 has UNIPROT Accession number 060462.

The term “a Semaphorin” are a class of secreted and membrane proteins that are grouped into eight major classes. Classes 3, 4, 6, and 7 are found in vertebrates only and lass 5 is found in both vertebrates and invertebrates. In humans, the genes encoding a Semaphorin are SEMA3A, SEMA3B, SEMA3C, SEMA3D, SEMA3E, SEMA3F, SEMA3GSEMA4A, SEMA4B, SEMA4C (“SEMAF”), SEMA4D, SEMA4F, SEMA4G, SEMA5A, SEMA5B, SEMA6A, SEMA6B, SEMA6C, SEMA6D, and SEMA7A.

The term “Semaphorin 3A” refers to the Semaphorin encoded in humans by the SEMA3A gene.

The term “Semaphorin 4A” refers to the Semaphorin encoded in humans by the SEMA4A gene.

The term “Semaphorin 6A” refers to the Semaphorin encoded in humans by the SEMA6A gene.

The term “PLP” refers to the “Myelin proteolipid protein”.

The term “Aquaporin-4” refers to a water channel protein encoded, in humans, by the AQP4 gene. The protein is also known as “AQP-4”.

The term “glutamate receptor” refers to a glutamate receptor and includes glutamate receptor 1 and glutamate receptor 2, but is not limited thereto.

The term “EpCAM” refers to the protein also known as “Epithelial cell adhesion molecule”.

“A second antigen-binding domain” of the molecule in the present invention may have any structure as long as it specifically binds “a second antigen” as described above. The structure of “a second antigen-binding domain” may include but is not limited to, a polypeptide or a portion thereof, or a small or medium chemical compound or a portion thereof, or a polynucleotide or a portion thereof. The polypeptide or a portion thereof includes but is not limited to a cell membrane protein expressed on a cell (e.g., an immune cell such as a dendritic cell) or a portion thereof (e.g., an extracellular domain, any unique domain thereof); an antibody (including but not limited to a human antibody, a chimeric, antibody, a humanized antibody, and VHH antibody) or an antigen-binding domain (also referred as a portion, a part or a fragment of an antibody). The antigen-binding domain of an antibody includes but is not limited to an antibody heavy chain variable (VH) region, an antibody light chain variable (VL) region (preferably a combination of an antibody heavy chain variable (VH) region and an antibody light chain variable (VL) region), a single-domain antibody (sdAb), a single-chain Fv (scFv), a single-chain antibody, a Fv, a single-chain Fv2 (scFv2), a Fab, and F (ab′)2.

The polypeptide or a portion thereof may also be an antigen binding polypeptides such as a module called A domain of Avimer, which has approximately 35 amino acids contained in an in vivo cell membrane protein (WO2004/044011 and WO2005/040229), adnectin having a 10Fn3 domain serving as a protein binding domain, which is derived from a glycoprotein fibronectin expressed on cell membranes (WO2002/032925), Affibody having an IgG binding domain scaffold constituting a three-helix bundle composed of 58 amino acids of protein A (WO1995/001937), DARPins (designed ankyrin repeat proteins) which are molecular surface-exposed regions of ankyrin repeats (AR) each having a 33-amino acid residue structure folded into a subunit of a turn, two antiparallel helices, and a loop (WO2002/020565), anticalin having four loop regions connecting eight antiparallel strands bent toward the central axis in one end of a barrel structure highly conserved in lipocalin molecules such as neutrophil gelatinase-associated lipocalin (NGAL) (WO2003/029462), and a depressed region in the internal parallel sheet structure of a horseshoe-shaped fold composed of repeated leucine-rich-repeat (LRR) modules of an immunoglobulin structure-free variable lymphocyte receptor (VLR) as seen in the acquired immune systems of jawless vertebrates such as lamprey or hagfish (WO2008/016854).

One embodiment of a polypeptide or a portion thereof which belongs to “a second antigen-binding domain” as described above in the present invention includes but is not limited to a cell membrane protein expressed on a cell (e.g., a receptor) or a portion thereof (e.g., an extracellular domain, any unique domain thereof).

The polypeptide or a portion thereof includes but is not limited to an antibody (including but not limited to a human antibody, a chimeric, antibody, a humanized antibody, and VHH antibody) or an antigen-binding domain (also referred as a portion, a part or a fragment of an antibody). The antigen-binding domain of an antibody includes but is not limited to an antibody heavy chain variable (VH) region, an antibody light chain variable (VL) region (preferably a combination of an antibody heavy chain variable (VH) region and an antibody light chain variable (VL) region), a single-domain antibody (sdAb), a VHH, a single-chain Fv (scFv), a single-chain antibody, a Fv, a single-chain Fv2 (scFv2), a Fab, a single chain Fab (scFab), and F (ab′)2.

### An Antigen-Binding Molecule Comprising a First Antigen-Binding Domain and a Second Antigen-Binding Domain

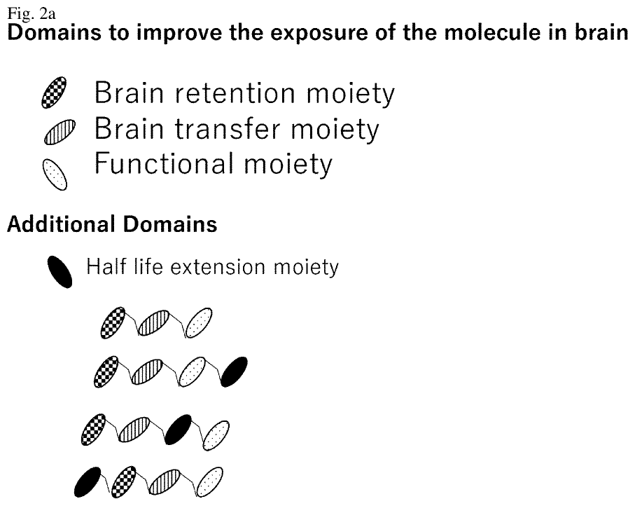

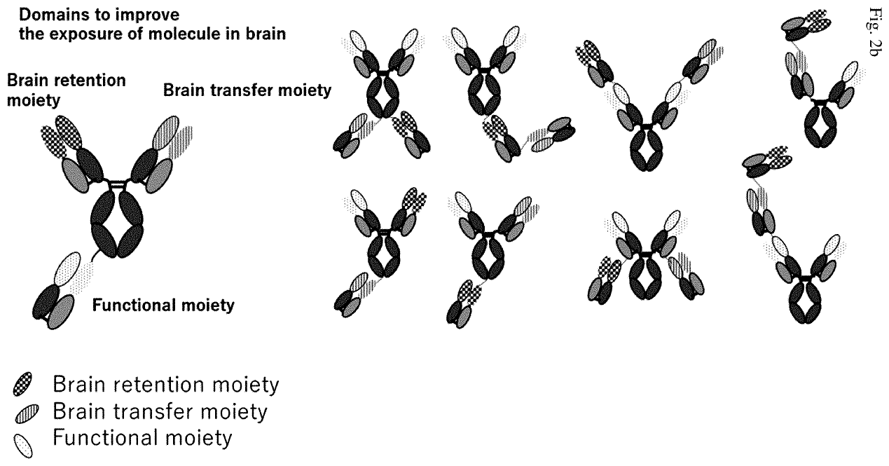

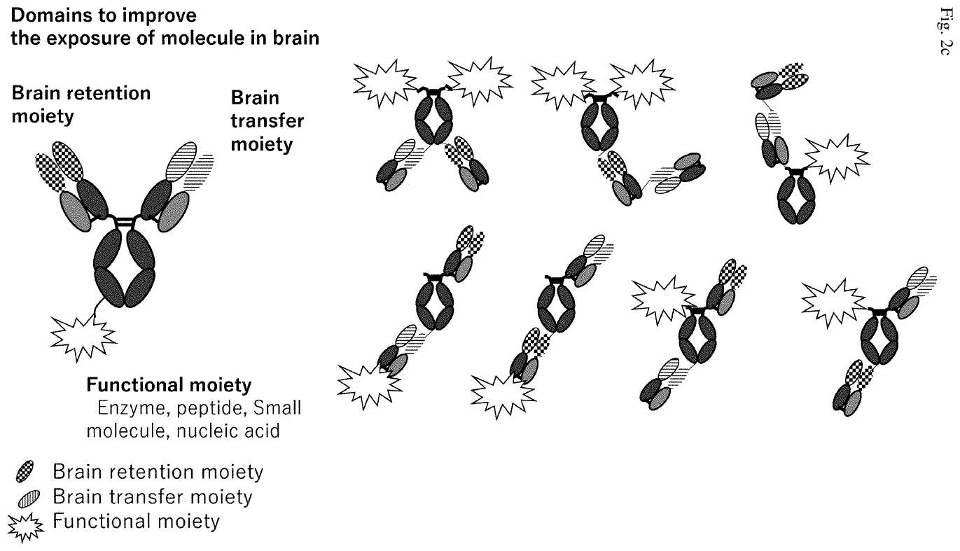

In the present invention the term “An antigen-binding molecule comprising a first antigen-binding domain and a second antigen-binding domain” includes molecules comprising at least one first antigen-binding domain and at least one second antigen-binding domain, wherein the domains may be linked covalently or non-covalently. The term includes but is not limited to a molecule having the following exemplary features.
i. An antibody which “a first antigen-binding domain” has been conjugated to a Fc region, and at least one of the two Fabs binds “a second antigen”. Alternatively, “a first antigen-binding domain” may be conjugated to one of Fab arms instead of, or in addition to the conjugation to Fc region. Further, more than two “a first antigen-binding domain” may be conjugated to an Fc region. The two Fab arms can conjugated to Fc region in various formats as illustrated in FIGS. 2(b) and 2(c). Alternatively to Fab arms, the antigen-binding domains of one or both Fab arms may have a different format, such as VHH, scFv or scFab.
ii. An antibody which one Fab arm binds “a first antigen” and another Fab arm binds “a second antigen”. One example of this embodiment may be a bispecific antibody or a bispecific antigen-binding domain, which one Fab arm binds a first target that facilitates transfer of the antigen-binding molecule into a mammalian brain as described above, and another Fab arm binds a second target wherein the second target (i) is expressed on the cell membrane of brain cells or (ii) is a brain ECM protein or is a brain ECM polysaccharide. The antibody or antibody-like molecule in this example can have various formats as illustrated in FIG. 2(b).
iii. A polypeptide comprising “a first antigen-binding moiety” and “a second antigen-binding moiety”, in which the first antigen-binding moiety and the second antigen-binding moiety are fused with or without a linker. One example of this embodiment is a fusion protein comprising a VHH or scFv binding to TfR and a VHH or scFv binding to MOG fused optionally via a peptide linker. Some of the examples in FIGS. 2(b) and 2(c) illustrate such embodiment.
iv. A chemical compound comprising “first antigen-binding domain” and “second antigen-binding domain”. One example of this embodiment may be a chemical compound comprising at least two moieties, which one moiety binds a first target that facilitates transfer of the antigen-binding molecule into a mammalian brain as described above, and the other moiety arm binds a second target wherein the second target (i) is expressed on the cell membrane of brain cells, or (ii) is a brain ECM protein or is a brain ECM polysaccharide. Such chemical compounds may be carried out by using a bioconjugate technique (Bioconjugate Techniques, 3rd Edition, 2013, by Greg T. Hermanson). The linker or compound may, for example, be further comprising a photoreactive group (e.g., aryl azide, diazirine, psoralen).
   The Antigen-Binding Molecule Further Comprising at Least One Functional Moiety and/or at Least One In Vivo Half-Life Extension Moiety

In embodiments, the antigen-binding molecule of any of the aspects and embodiments herein further comprises at least one functional moiety and/or at least one in vivo half-life extension moiety.

In embodiments, the antigen-binding molecule of any of the aspects and embodiments herein further comprises
(i) at least one functional moiety comprising an enzyme, a therapeutic protein, an antibody or antigen-binding fragment thereof, a peptide, a DNA, an shRNA, an siRNA, a small molecule drug, or a cytotoxic agent, and/or
(ii) at least one in vivo half-life extension moiety, preferably wherein said at least one in vivo half-life extension moiety is selected from the group consisting of an Fc region, an albumin-binding domain, an FcRn-binding protein, an FcRn-binding peptide and a PEG moiety.

In some embodiments, the antigen-binding molecule of any of the aspects and embodiments herein further comprises at least one functional moiety. In embodiments, the functional moiety is selected from the group consisting of an enzyme, a therapeutic protein, an antibody or antigen-binding fragment thereof, a peptide, a DNA, an shRNA, an siRNA, a small molecule drug, and a cytotoxic agent.

In certain embodiments, the functional moiety is not a Fc region.

Exemplary embodiments for formats of an antigen-binding molecule further comprising at least one functional moiety are depicted in FIGS. 2(a) and 2(c).

In embodiments, the antigen-binding molecule of any of the aspects and embodiments herein further comprises at least one in vivo half-life extension moiety. In embodiments, the in vivo half-life extension moiety is selected from the group consisting of an Fc region, an albumin-binding domain, an FcRn-binding protein, an FcRn-binding peptide and a PEG moiety.

Exemplary embodiments for formats of an antigen-binding molecule further comprising an Fc region as exemplary in vivo half-life extension moiety are depicted in FIGS. 2(a) and 2(c).

In embodiments, the antigen-binding molecule of any of the aspects and embodiments herein further comprises at least one functional moiety and at least one in vivo half-life extension moiety. Exemplary embodiments for formats of an antigen-binding molecule further comprising at least one functional moiety and an Fc region as exemplary in vivo half-life extension moiety are depicted in FIGS. 2(a) and 2(c).

The term “in vivo half-life” of a molecule is understood as the time it takes for the concentration of the molecule in a given tissue to halve (half-life) its steady-state when present in the respect tissue of a subject. In embodiments, the subject is a mammal. In embodiments, the subject is a human. Methods for determining in vivo half-life are known in the art and a suitable method is described in the examples.

The term “in vivo half-life extension moiety” is understood as a moiety which, which when linked to an active agent, extends the in vivo half-life in blood as compared to the active agent without such moiety. In embodiments, in vivo half-life extension moiety” is understood as a moiety which, which when linked to an active agent, extends the in vivo half-life by at least 10%, 20%, 30%, 40%, 50%, 100%, 200%, 500% or 1000%.

Suitable in vivo half-life extension moieties are known in the art and can be used. In embodiments, an Fc region, an albumin-binding domain, an FcRn-binding protein, an FcRn-binding peptide or a PEG moiety may be used. For example, in the case of an Fc region, the Fc region is capable of binding to one or more Fc receptors. In particular, in the case of an Fc region, the Fc region is capable of binding to the FcRn. Such Fc regions are suitable for extending in vivo half-life.

In embodiments, the antigen-binding molecule of any of the aspects or embodiments is an Fc region-comprising antibody.

In other embodiments, the in vivo half-life extension moiety is an FcRn-binding protein or an FcRn-binding peptide. Such moieties are known in the art and are for example disclosed in US20170275373 and Datta-Mannan A et al (Biotechnol J. 2019 March; 14(3):e1800007. doi: 10.1002/biot.201800007).

A “PEG” moiety is understood as moiety comprising two or more polyethylene glycol moieties which are covalently linked to each other. For example, PEG moieties may be used which have a molecular weight of between 500 Da to 100 kDa, such as between 1000 Da to 50000 da. “Albumin-binding domains” are understood as moieties which are capable of binding to mammalian albumin. In embodiments, mammalian albumin is human albumin. The structure of an Albumin-binding domain is not limited and includes for example proteins, peptides and small molecules. In embodiments, the Albumin-binding domain is a peptide or protein. Suitable moieties are known in the art and are for example described in Tan H et al. (Eur J Pharmacol. 2021 Jan. 5; 890:173650).

A “functional moiety” is understood as a moiety which is capable of exerting a therapeutic, preventive and/or diagnostic effect. The skilled person is aware of such “functional moiety” and can select such “functional moiety” depending on the disease or disorder to be treated, prevented and/or diagnosed. In embodiments, the functional moiety comprises an enzyme, a therapeutic protein, an antibody or antigen-binding fragment thereof, a peptide, a DNA, an shRNA, an siRNA, a small molecule drug, or a cytotoxic agent. The structure of the functional moiety is not limited as long as the functional moiety exerts the desired therapeutic, preventive or diagnostic effect.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half-life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Region-Comprising Antibody

The term “Fc region-comprising antibody” refers to an antibody that comprises an Fc region. The C-terminal lysine (residue 447 according to the EU numbering system) or C-terminal glycine-lysine (residues 446-447) of the Fc region may be removed, for example, during purification of the antibody or by recombinant engineering of the nucleic acid encoding the antibody. Accordingly, a composition comprising an antibody having an Fe region according to this invention can comprise an antibody with G446-K447, with G446 and without K447, with all G446-K447 removed, or a mixture of three types of antibodies described above.

### A Method for Increasing the Concentration of an Antigen-Binding Molecule in the Brain, a Method for Increasing the Exposure of an Antigen-Binding Molecule in the Brain and a Method for the Retention of an Antigen-Binding Molecule in the Brain

In one aspect, a method for increasing the concentration of an antigen-binding molecule in the brain of a subject in need thereof is provided, the method comprising:
(a) providing a first antigen-binding molecule comprising:
(a1) a first antigen-binding domain that specifically binds a first target that facilitates transfer of the antigen-binding molecule into a mammalian brain; or
(a2) a second antigen-binding domain that specifically binds a second target wherein the second target (i) is expressed on the cell membrane of brain cells, or (ii) is a brain ECM protein or a brain ECM polysaccharide;
(b) introducing
in the case of (a1), to the first antigen-binding molecule at least one second antigen-binding domain that specifically binds the second target wherein the second target (i) is expressed on the cell membrane of brain cells, or (ii) is a brain ECM protein or a brain ECM polysaccharide, thereby obtaining a second antigen-binding molecule; or
in the case of (a2), to the first antigen-binding molecule at least one first antigen-binding domain that specifically binds the first target that facilitates transfer of the antigen-binding molecule into a mammalian brain, thereby obtaining a second antigen-binding molecule;
such that the concentration of said second antigen-binding molecule in the brain of said subject is increased upon administration to said subject as compared to the first antigen-binding molecule.

In some embodiments, “concentration of an antigen-binding molecule in the brain of a subject” is understood as concentration of the antigen-binding molecule in brain per brain tissue weight and is typically provided in unit microgram/g brain. The concentration can be determined as follows (here provided for an “antibody” as exemplary antigen-binding molecule:

$$Antibody concentration ( micro gram per gram brain ) = \frac{the amount of antibody ( micro gram )}{brain tissue weight ( gram )}$$

In some further embodiments, the term “increasing the concentration of an antigen-binding molecule in the brain of a subject” is understood as increasing the concentration (micro gram per gram brain) by 1% or more, 5% or more, 10% or more, 15% or more, 20%, 30%, 40% 50%, 60%, 70%, 80%, 90%, 100%, or 200% or more as compared to the first antigen-binding molecule.

In some embodiments, “concentration of an antigen-binding molecule in the brain of a subject” is understood as the percent value of injected dose per brain tissue weight and is typically provided in % ID (injected dose)/g brain, which can be determined as follows (here provided for an “antibody” as exemplary antigen-binding molecule:

$$Antibody concentration ( \% ID per gram brain ) = \frac{\begin{matrix}the antibody concentration in brain \\ ( micro gram per gram brain )\end{matrix}}{the injected dose amount of antibody ( micro gram )} \times 100$$

In some further embodiments, the term “increasing the concentration of an antigen-binding molecule in the brain of a subject” is understood as increasing the concentration (% ID/g brain) by 1% or more, 5% or more, 10% or more, 15% or more, 20%, 30%, 40% 50%, 60%, 70%, 80%, 90%, 100%, or 200% or more as compared to the first antigen-binding molecule.

In embodiments, a method for increasing the concentration of an antigen-binding molecule in the brain of a subject in need thereof is provided, the method comprising:
(a) providing a first antigen-binding molecule comprising:
a first antigen-binding domain that specifically binds a first target that facilitates transfer of the antigen-binding molecule into a mammalian brain;
(b) introducing
to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule;
such that the concentration of said second antigen-binding molecule in the brain of said subject is increased upon administration to said subject as compared to the first antigen-binding molecule.

In other embodiments, a method for increasing the concentration of an antigen-binding molecule in the brain of a subject in need thereof is provided, the method comprising:
(a) providing a first antigen-binding molecule comprising:
a second antigen-binding domain that specifically binds a second target wherein the second target (i) is expressed on the cell membrane of brain cells, or (ii) is a brain ECM protein or a brain ECM polysaccharide;
(b) introducing
to the first antigen-binding molecule at least one first antigen-binding domain that specifically binds a first target that facilitates transfer of the antigen-binding molecule into a mammalian brain, thereby obtaining a second antigen-binding molecule;
such that the concentration of said second antigen-binding molecule in the brain of said subject is increased upon administration to said subject as compared to the first antigen-binding molecule.

For the “first antigen-binding domain”, the “first target”, the “second antigen-binding domain” and the “second target”, the same embodiments apply as for the “antigen-binding molecules” of any of the aspects and embodiments above.

The step of “introducing” in (b) may be achieved by methods known to a skilled person, such as by covalently linking the “second antigen-binding domain” or “first antigen-binding domain” to the “first antigen-binding molecule”, optionally wherein a linker is used. For example, the step of “introducing” may encompass generating one or more nucleic acid(s) encoding the second antigen-binding molecule and obtaining the second antigen-binding molecule by recombinant expression techniques.

In embodiments, the second antigen-binding molecule is as defined in any of the aspects and embodiments of an antigen-binding molecule of the invention.

In certain embodiments, the method further comprising step (c):
(c) determining that the concentration of said second antigen-binding molecule in the brain of said subject is increased compared to a control antigen-binding molecule, wherein the control antigen-binding molecule differs from the second antigen-binding molecule according to (b) only in that it does not comprise:
in the case of (a1), said at least one second antigen-binding domain that specifically binds the second target wherein the second target (i) is expressed on the cell membrane of brain cells, or (ii) is a brain ECM protein or a brain ECM polysaccharide; or
in the case of (a2) said at least one first antigen-binding domain that specifically binds the first target that facilitates transfer of the antigen-binding molecule into a mammalian brain.

The concentration of a control may be determined at the same time point or at a different point. In one embodiment, the concentration of a control may be determined at the same time point.

The “control antigen-binding molecule” therefore, in the case of (a1) does not contain said at least one second antigen-binding domain that specifically binds a second target; or in the case of (a2) does not contain said at least one first antigen-binding domain that specifically binds a first target. The remaining parts remain identical.

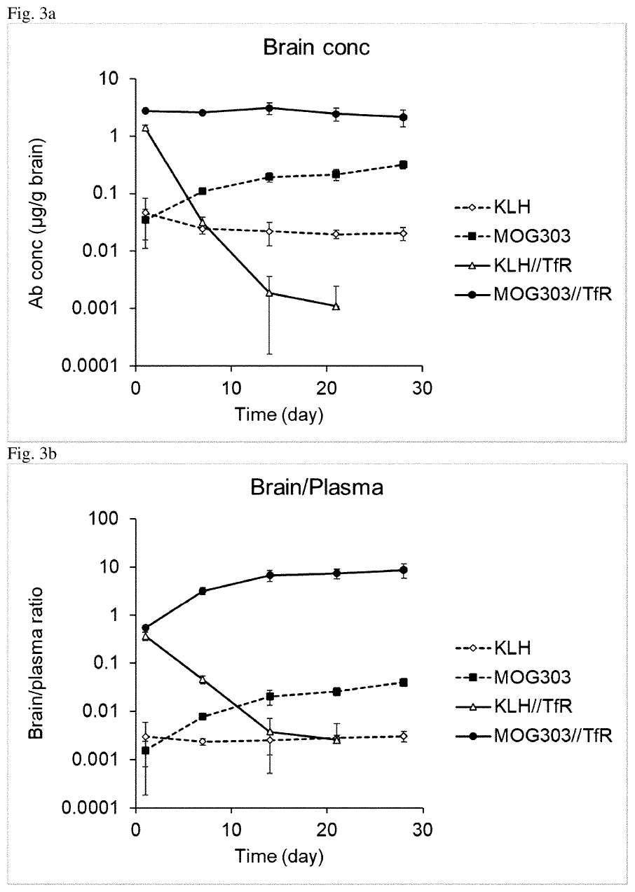

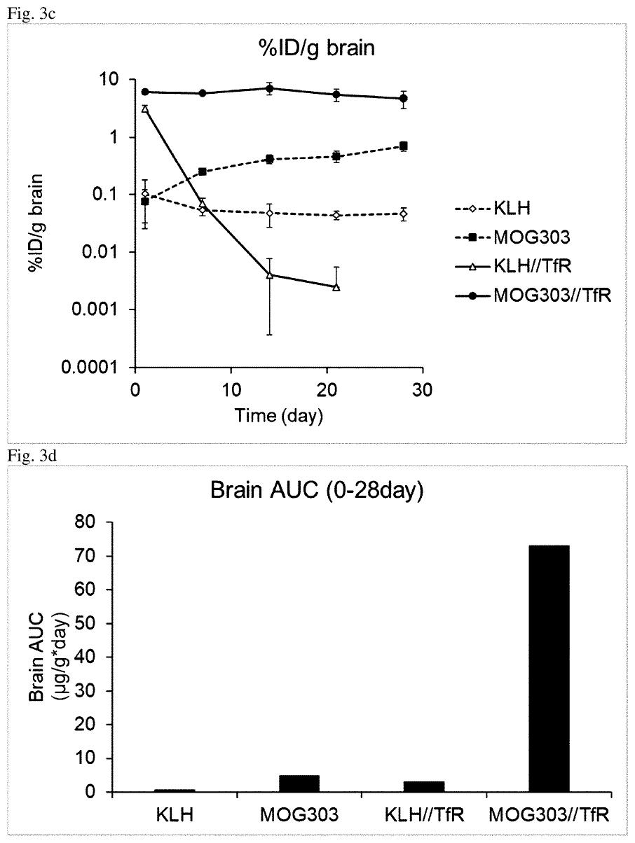

It was found in the Examples 3, 5, 6 and 7 and in FIGS. 3(c), 6(c), 8(c) and 10(c) that the concentration of bispecific antibodies comprising a brain transfer moiety and a brain retention moiety increased in the brain as determined as % ID/g brain, as compared to a control antibody comprising only a brain transfer moiety or only a brain retention moiety. For example, anti-Basigin/anti-MOG bispecific antibodies, anti-TfR/anti-MOG bispecific antibodies and an anti-TfR/anti-CADM3 bispecific antibody are provided in Examples 4 and 5. It was surprisingly found that the combination of the brain transfer moiety and the brain retention moiety in these bispecific antibodies showed much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain. Moreover, an anti-TfR/anti-CSPG5 bispecific antibody is provided in Examples 4 and 7. The results in Example 7 indicate that the combination of a brain transfer moiety binding to TfR and brain retention moiety binding to CSPG5 has superior potency in the transfer and retention into the brain as compared to an antibody that comprises a moiety binding to CSPG5 only.

In embodiments, the concentration of said second antigen-binding molecule in the brain of said subject is Cmax. “Cmax” is also known as Maximum Concentration and is referred to as the highest antibody concentration in the brain during the period of time after administration.

In an embodiment herein, the mammalian brain is human brain. Accordingly, in one embodiment, the first target facilitates transfer of the antigen-binding molecule into a human brain. Methods for determining Cmax are known in the art. A suitable method for determining Cmax is described in the Examples.

For example, it was shown in Example 3 that cmax in brain of a bispecific antibody comprising a brain transfer moiety and a brain retention moiety (anti-TfR/anti-MOG bispecific antibody) is higher than cmax in brain of a bispecific antibody comprising a brain transfer moiety only (anti-TfR/anti-KLH).

In further embodiments, the first target is as specified in any one of the embodiments of an antigen-binding molecule of the first aspect herein. In further embodiments, the second target is as specified in any one of the embodiments of an antigen-binding molecule of the first aspect herein. In further embodiments, the first target and the second target are as specified in any one of the embodiments of an antigen-binding molecule of the first aspect herein.

In particular, in further embodiments, the first target and the second target are as follows:
(i) the first target is Transferrin receptor (TfR) and the second target is Myelin Oligodendrocyte glycoprotein (MOG);
(ii) the first target is Transferrin receptor (TfR) and the second target is Neuroglycan C/Chondroitin sulfate proteoglycan 5 (CSPG5);
(iii) the first target is Transferrin receptor (TfR) and the second target is Cell Adhesion Molecule 3 (CADM3);
(iv) the first target is Basigin (CD147) and the second target is Myelin Oligodendrocyte glycoprotein (MOG);
(v) the first target is Basigin (CD147) and the second target is Neuroglycan C/Chondroitin sulfate proteoglycan 5 (CSPG5); or
(vi) the first target is Basigin (CD147) and the second target is Cell Adhesion Molecule 3 (CADM3);
(vii) the first target is Insulin receptor and the second target is Myelin Oligodendrocyte glycoprotein (MOG);
(viii) the first target is Insulin receptor and the second target is Neuroglycan C/Chondroitin sulfate proteoglycan 5 (CSPG5);
(ix) the first target is Insulin receptor and the second target is Cell Adhesion Molecule 3 (CADM3);
(x) the first target is glucose receptor and the second target is Myelin Oligodendrocyte glycoprotein (MOG);
(xi) the first target is glucose receptor and the second target is Neuroglycan C/Chondroitin sulfate proteoglycan 5 (CSPG5);
(xii) the first target is glucose receptor and the second target is Cell Adhesion Molecule 3 (CADM3);
(xiii) the first target is Low density lipoprotein Receptor (LDLR) and the second target is Myelin Oligodendrocyte glycoprotein (MOG);
(xiv) the first target is Low density lipoprotein Receptor (LDLR) and the second target is Neuroglycan C/Chondroitin sulfate proteoglycan 5 (CSPG5); or
(xv) the first target is Low density lipoprotein Receptor (LDLR) and the second target is Cell Adhesion Molecule 3 (CADM3);
(xii) the first target is Low density lipoprotein receptor related protein (LRP) and the second target is Myelin Oligodendrocyte glycoprotein (MOG);
(xii) the first target is Low density lipoprotein receptor related protein (LRP) and the second target is Neuroglycan C/Chondroitin sulfate proteoglycan 5 (CSPG5); or
(xviii) the first target is Low density lipoprotein receptor related protein (LRP) and the second target is Cell Adhesion Molecule 3 (CADM3); or
(xix) the first target is CD98hc and the second target is Myelin Oligodendrocyte glycoprotein (MOG); or
(xx) the first target is CD98hc and the second target is Neuroglycan C/Chondroitin sulfate proteoglycan 5 (CSPG5); or
(xxi) the first target is CD98hc and the second target is Cell Adhesion Molecule 3 (CADM3).

In particular, the second antigen-binding molecule is an antigen-binding molecule according to any of the embodiments of an antigen-binding molecule of the first aspect herein.

In a further aspect, a method is provided for increasing exposure of an antigen-binding molecule in the brain of a subject in need thereof, the method comprising:
(a) providing a first antigen-binding molecule comprising:
(a1) a first antigen-binding domain that specifically binds a first target that facilitates transfer of the antigen-binding molecule into a mammalian brain; or
(a2) a second antigen-binding domain that specifically binds a second target wherein the second target (i) is expressed on the cell membrane of brain cells, or (ii) is a brain ECM protein or a brain ECM polysaccharide;
(b) introducing
in the case of (a1), to the first antigen-binding molecule at least one second antigen-binding domain that specifically binds the second target wherein the second target (i) is expressed on the cell membrane of brain cells, or (ii) is a brain ECM protein or a brain ECM polysaccharide, thereby obtaining a second antigen-binding molecule; or
in the case of (a2), to the first antigen-binding molecule at least one first antigen-binding domain that specifically binds the first target that facilitates transfer of the antigen-binding molecule into a mammalian brain, thereby obtaining a second antigen-binding molecule;
such that the exposure of said second antigen-binding molecule in the brain of said subject is increased upon administration to said subject as compared to the first antigen-binding molecule.

The term “exposure of said second antigen-binding molecule in the brain of said subject” is understood as Area Under Curve (AUC) in brain. “Area Under Curve” or “AUC” or “AUC (Area Under Curve) of brain concentration-time profiles of the antigen-binding molecule” are used herein interchangeably and correspond to the cumulative exposure of the antibody in brain during the period of study. AUC may be determined by pharmacokinetic methods known in the art, such as by measuring the concentration of the antigen-binding molecule of interest at a plurality of discrete points in time and using the trapezoidal rule to estimate and thereby determine AUC. A suitable method for determining AUC is described in the Examples.

The term “increasing the exposure of an antigen-binding molecule in the brain of a subject” is understood as increasing the exposure by 1% or more, 5% or more, 10% or more, 15% or more, 20%, 30%, 40% 50%, 60%, 70%, 80%, 90%, 100%, or 200% or more as compared to the first antigen-binding molecule.

For the “first antigen-binding domain”, the “first target”, the “second antigen-binding domain” and the “second target”, the same embodiments apply as for the “antigen-binding molecules” of any of the aspects and embodiments above.

The step of “introducing” in (b) may be achieved by methods known to a skilled person, such as by covalently linking the “second antigen-binding domain” or “first antigen-binding domain” to the “first antigen-binding molecule”, optionally wherein a linker is used. For example, the step of “introducing” may encompass generating one or more nucleic acid(s) encoding the second antigen-binding molecule and obtaining the second antigen-binding molecule by recombinant expression techniques.

In embodiments, the second antigen-binding molecule is as defined in any of the aspects and embodiments of an antigen-binding molecule of the invention.

In an embodiment, a method is provided for increasing exposure of an antigen-binding molecule in the brain of a subject in need thereof, the method comprising:
(a) providing a first antigen-binding molecule comprising:
a first antigen-binding domain that specifically binds a first target that facilitates transfer of the antigen-binding molecule into a mammalian brain,
(b) introducing
to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule;
such that the exposure of said second antigen-binding molecule in the brain of said subject is increased upon administration to said subject as compared to the first antigen-binding molecule.

In an embodiment, a method is provided for increasing exposure of an antigen-binding molecule in the brain of a subject in need thereof, the method comprising:
(a) providing a first antigen-binding molecule comprising:
a second antigen-binding domain that specifically binds a second target wherein the second target (i) is expressed on the cell membrane of brain cells, or (ii) is a brain ECM protein or a brain ECM polysaccharide;
(b) introducing
to the first antigen-binding molecule at least one first antigen-binding domain that specifically binds a first target that facilitates transfer of the antigen-binding molecule into a mammalian brain, thereby obtaining a second antigen-binding molecule;
such that the exposure of said second antigen-binding molecule in the brain of said subject is increased upon administration to said subject as compared to the first antigen-binding molecule.

### In an Embodiment, the Method Further Comprises Step (c):

(c) determining that the exposure of said second antigen-binding molecule in the brain of said subject is increased compared to a control antigen-binding molecule, wherein the control antigen-binding molecule differs from the second antigen-binding molecule according to (b) only in that it does not comprise
in the case of (a1), said at least one second antigen-binding domain that specifically binds to the second target wherein the second target (i) is expressed on the cell membrane of brain cells, or (ii) is a brain ECM protein or a brain ECM polysaccharide; or
in the case of (a2) said at least one first antigen-binding domain that specifically binds the first target that facilitates transfer of the antigen-binding molecule into a mammalian brain.

In an embodiment, the exposure of said second antigen-binding molecule in the brain of said subject is the AUC (Area Under Curve) of brain concentration-time profiles of the antigen-binding molecule.

In an embodiment herein, the mammalian brain is human brain. Accordingly, in one embodiment, the first target facilitates transfer of the antigen-binding molecule into a human brain.

In further embodiments, the first target is as specified in any one of the embodiments of an antigen-binding molecule of the first aspect herein. In further embodiments, the second target is as specified in any one of the embodiments of an antigen-binding molecule of the first aspect herein. In further embodiments, the first target and the second target are as specified in any one of the embodiments of an antigen-binding molecule of the first aspect herein.

In particular, in further embodiments, the first target and the second target are as follows:
(i) the first target is Transferrin receptor (TfR) and the second target is Myelin Oligodendrocyte glycoprotein (MOG);
(ii) the first target is Transferrin receptor (TfR) and the second target is Neuroglycan C/Chondroitin sulfate proteoglycan 5 (CSPG5);
(iii) the first target is Transferrin receptor (TfR) and the second target is Cell Adhesion Molecule 3 (CADM3);
(iv) the first target is Basigin (CD147) and the second target is Myelin Oligodendrocyte glycoprotein (MOG);
(v) the first target is Basigin (CD147) and the second target is Neuroglycan C/Chondroitin sulfate proteoglycan 5 (CSPG5); or
(vi) the first target is Basigin (CD147) and the second target is Cell Adhesion Molecule 3 (CADM3);
(vii) the first target is Insulin receptor and the second target is Myelin Oligodendrocyte glycoprotein (MOG);
(viii) the first target is Insulin receptor and the second target is Neuroglycan C/Chondroitin sulfate proteoglycan 5 (CSPG5);
(ix) the first target is Insulin receptor and the second target is Cell Adhesion Molecule 3 (CADM3);
(x) the first target is glucose receptor and the second target is Myelin Oligodendrocyte glycoprotein (MOG);
(xi) the first target is glucose receptor and the second target is Neuroglycan C/Chondroitin sulfate proteoglycan 5 (CSPG5);
(xii) the first target is glucose receptor and the second target is Cell Adhesion Molecule 3 (CADM3);
(xiii) the first target is Low density lipoprotein Receptor (LDLR) and the second target is Myelin Oligodendrocyte glycoprotein (MOG);
(xiv) the first target is Low density lipoprotein Receptor (LDLR) and the second target is Neuroglycan C/Chondroitin sulfate proteoglycan 5 (CSPG5); or
(xv) the first target is Low density lipoprotein Receptor (LDLR) and the second target is Cell Adhesion Molecule 3 (CADM3);
(xii) the first target is Low density lipoprotein receptor related protein (LRP) and the second target is Myelin Oligodendrocyte glycoprotein (MOG);
(xii) the first target is Low density lipoprotein receptor related protein (LRP) and the second target is Neuroglycan C/Chondroitin sulfate proteoglycan 5 (CSPG5); or
(xviii) the first target is Low density lipoprotein receptor related protein (LRP) and the second target is Cell Adhesion Molecule 3 (CADM3); or
(xix) the first target is CD98hc and the second target is Myelin Oligodendrocyte glycoprotein (MOG); or
(xx) the first target is CD98hc and the second target is Neuroglycan C/Chondroitin sulfate proteoglycan 5 (CSPG5); or.
(xxi) the first target is CD98hc and the second target is Cell Adhesion Molecule 3 (CADM3).

In particular, the second antigen-binding molecule is an antigen-binding molecule according to any of the embodiments of an antigen-binding molecule of the first aspect herein.

It was found in the Example 3 in FIG. 3(d) that the exposure, determined as AUC, of bispecific antibodies comprising a brain transfer moiety and a brain retention moiety is increased in the brain as compared to a control antibody comprising only a brain transfer moiety or only a brain retention moiety. In particular, unexpectedly, MOG303/TfR showed synergistic effect in terms of cumulative brain AUC (area under the curve) which represents the total antibody exposure in brain across a time interval, wherein the cumulative brain AUC of MOG303/TfR is approximately 108-fold, 15.2-fold, and 24.7-fold higher than that of KLH, MOG303, and KLH/TfR, respectively (FIG. 3(d)). Notably, the cumulative brain AUC of MOG303/TfR (73.0 microgram/g*day) is greater than the sum of cumulative brain AUC of each of MOG303 (4.80 microgram/g*day) and KLH/TfR (2.95 microgram/g*day). The results suggest that the combination of the transfer moiety (anti-TfR) and retention moiety (anti-MOG) in a bispecific antibody provides synergistic effect of longer lasting and high antibody concentration in brain.

In an aspect, a method is provided for the retention of an antigen-binding molecule in the brain of a subject in need thereof, the method comprising:
(a) providing a first antigen-binding molecule comprising a first antigen-binding domain that specifically binds a first target that facilitates transfer of the antigen-binding molecule into a mammalian brain;
(b) introducing to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule,
such that the retention of said second antigen-binding molecule in the brain of said subject is increased upon administration to said subject as compared to the first antigen-binding molecule.

In particular, the data in Examples 3, 5, 6 and 7 for an anti-TfR/anti-CSPG5 bispecific antibody, anti-Basigin/anti-MOG bispecific antibodies, anti-TfR/anti-MOG bispecific antibodies and an anti-TfR/anti-CADM3 bispecific antibody suggest that the combination of the brain transfer moiety and the brain retention moiety has a synergistic effect on the potency of retention of an antibody in brain.

The term “retention” or “retention of an antigen-binding molecule in the brain of a subject” is understood as in vivo half-live in brain of a subject.

For example, it was shown in Example 3 and FIG. 3 that in vivo half-live in brain of a subject of a bispecific antibody comprising a brain transfer moiety and a brain retention moiety (anti-TfR/anti-MOG bispecific antibody) is higher than for a bispecific antibody comprising a brain transfer moiety only (anti-TfR/anti-KLH).

The term “in vivo half-life in brain” of a molecule is understood as the time it takes for the concentration of the molecule in brain to halve (half-life) its steady-state when present in brain of a subject. In embodiments, the subject is a mammal. In embodiments, the subject is a human. In further embodiments, the “retention of said second antigen-binding molecule in the brain of said subject” is the half-life in vivo in the brain.

The term “increasing the retention of an antigen-binding molecule in the brain of a subject” is understood as increasing the retention in brain, or in vivo half-life in brain, by 1% or more, 5% or more, 10% or more, 15% or more, 20%, 30%, 40% 50%, 60%, 70%, 80%, 90%, 100%, or 200% or more as compared to the first antigen-binding molecule.

In embodiments, the half-life in vivo in the brain of said second antigen-binding molecule is at least 10 days, 12 days, 15 days, 20 days, 30 days, 45 days, 60 days or 90 days. For example, the half-life in vivo in the brain of said second antigen-binding molecule is at least 10 days, 12 days, 15 days, 20 days, 30 days, 45 days, 60 days or 90 days or up to 100, 150 or 200 days, or any subrange thereof.

Accordingly, in embodiments, the in vivo half-live in brain, of said second antigen-binding molecule is increased by 1% or more, 5% or more, 10% or more, 15% or more, 20%, 30%, 40% 50%, 60%, 70%, 80%, 90%, 100%, or 200% or more as compared to the first antigen-binding molecule, and the half-life in vivo in the brain of said second antigen-binding molecule is at least 10 days, 12 days, 15 days, 20 days, 30 days, 45 days, 60 days or 90 days. For example, the half-life in vivo in the brain of said second antigen-binding molecule is at least 10 days, 12 days, 15 days, 20 days, 30 days, 45 days, 60 days or 90 days or up to 100, 150 or 200 days, or any subrange thereof.

For the “first antigen-binding domain”, the “first target”, the “second antigen-binding domain” and the “second target”, the same embodiments apply as for the “antigen-binding molecules” of any of the aspects and embodiments above.

The step of “introducing” in (b) may be achieved by methods known to a skilled person, such as by covalently linking the “second antigen-binding domain” to “first antigen-binding molecule”, optionally wherein a linker is used. For example, the step of “introducing” may encompass generating one or more nucleic acid(s) encoding the second antigen-binding molecule and obtaining the second antigen-binding molecule by recombinant expression techniques.

In embodiments, the second antigen-binding molecule is as defined in any of the aspects and embodiments of an antigen-binding molecule of the invention.

In an embodiment, the method further comprises step (c):
(c) determining that the retention of said second antigen-binding molecule in the brain of said subject is increased compared to a control antigen-binding molecule, wherein the control antigen-binding molecule differs from the second antigen-binding molecule according to (b) only in that it does not comprise said at least one antigen-binding domain that specifically binds a second target.

In an embodiment herein, the mammalian brain is human brain. Accordingly, in one embodiment, the first target facilitates transfer of the antigen-binding molecule into a human brain.

In further embodiments, the first target is as specified in any one of the embodiments of an antigen-binding molecule of the first aspect herein. In further embodiments, the second target is as specified in any one of the embodiments of an antigen-binding molecule of the first aspect herein. In further embodiments, the first target and the second target are as specified in any one of the embodiments of an antigen-binding molecule of the first aspect herein.

In particular, in further embodiments, the first target and the second target are as follows:
(i) the first target is Transferrin receptor (TfR) and the second target is Myelin Oligodendrocyte glycoprotein (MOG);
(ii) the first target is Transferrin receptor (TfR) and the second target is Neuroglycan C/Chondroitin sulfate proteoglycan 5 (CSPG5);
(iii) the first target is Transferrin receptor (TfR) and the second target is Cell Adhesion Molecule 3 (CADM3);
(iv) the first target is Basigin (CD147) and the second target is Myelin Oligodendrocyte glycoprotein (MOG);
(v) the first target is Basigin (CD147) and the second target is Neuroglycan C/Chondroitin sulfate proteoglycan 5 (CSPG5); or
(vi) the first target is Basigin (CD147) and the second target is Cell Adhesion Molecule 3 (CADM3);
(vii) the first target is Insulin receptor and the second target is Myelin Oligodendrocyte glycoprotein (MOG);
(viii) the first target is Insulin receptor and the second target is Neuroglycan C/Chondroitin sulfate proteoglycan 5 (CSPG5);
(ix) the first target is Insulin receptor and the second target is Cell Adhesion Molecule 3 (CADM3);
(x) the first target is glucose receptor and the second target is Myelin Oligodendrocyte glycoprotein (MOG);
(xi) the first target is glucose receptor and the second target is Neuroglycan C/Chondroitin sulfate proteoglycan 5 (CSPG5);
(xii) the first target is glucose receptor and the second target is Cell Adhesion Molecule 3 (CADM3);
(xiii) the first target is Low density lipoprotein Receptor (LDLR) and the second target is Myelin Oligodendrocyte glycoprotein (MOG);
(xiv) the first target is Low density lipoprotein Receptor (LDLR) and the second target is Neuroglycan C/Chondroitin sulfate proteoglycan 5 (CSPG5); or (xv) the first target is Low density lipoprotein Receptor (LDLR) and the second target is Cell Adhesion Molecule 3 (CADM3);
(xii) the first target is Low density lipoprotein receptor related protein (LRP) and the second target is Myelin Oligodendrocyte glycoprotein (MOG);
(xii) the first target is Low density lipoprotein receptor related protein (LRP) and the second target is Neuroglycan C/Chondroitin sulfate proteoglycan 5 (CSPG5); or
(xviii) the first target is Low density lipoprotein receptor related protein (LRP) and the second target is Cell Adhesion Molecule 3 (CADM3); or
(xix) the first target is CD98hc and the second target is Myelin Oligodendrocyte glycoprotein (MOG); or
(xx) the first target is CD98hc and the second target is Neuroglycan C/Chondroitin sulfate proteoglycan 5 (CSPG5); or
(xxi) the first target is CD98hc and the second target is Cell Adhesion Molecule 3 (CADM3).

In particular, the second antigen-binding molecule is an antigen-binding molecule according to any of the embodiments of an antigen-binding molecule of the first aspect herein.

The “brain-to-plasma ratio” is understood as the ratio of the concentration of the antigen-binding molecule in brain and the concentration of the antigen-binding molecule in plasma. The “brain-to-plasma ratio” can be calculated as follows: Brain to plasma ratio the antibody concentration in brain (micro gram per gram brain) the antibody concentration in plasma (micro gram per mL plasma)

$$Brain to plasma ratio = \frac{the antibody concentration in brain ( micro gram per gram brain )}{the antibody concentration in plasma ( micro gram per mL plasma )}$$

The “transfer efficiency” is understood as the percent value of injected dose per brain tissue weight (% ID (injected dose)/g brain). The “transfer efficiency” can be calculated as follows:

$$\% ID per gram brain = \frac{the antibody concentration in brain ( micro gram per gram brain )}{the injected dose amount of antibody ( micro gram )} \times 100$$

### Embodiments for the First Target and Second Target

As described above, for any of the aspects herein, the first antigen-binding domain specifically binds a first target that facilitates transfer of the antigen-binding molecule into a mammalian brain, and
the second antigen-binding domain specifically binds a second target wherein the second target (i) is expressed on the cell membrane of brain cells, or (ii) is a brain ECM protein or a brain ECM polysaccharide.

Embodiments for the first target are described above and the second target are described above. Further embodiments for any of the aspects herein are disclosed in the following.

According to certain embodiments, the second target is expressed on the cell membrane of brain cells, wherein the brain cells comprise one or more of the cells selected from the group consisting of oligodendrocytes, astrocytes, neurons and microglia. For example, the second target is expressed on the cell membrane of oligodendrocytes. Such second targets include for example Myelin Oligodendrocyte glycoprotein, CNPase, and MAG as shown in Table 2. For example, the second target is expressed on the cell membrane of astrocytes. Such second targets include for example EEAT1 and EEAT2 as shown in Table 2. For example, the second target is expressed on the cell membrane of microglia. Such second targets include for example P2RY12 and AIF1 as shown in Table 2. For example, the second target is expressed on the cell membrane of more than one brain cell. Such second targets include for example CSPG5 and SynCAM3 as shown in Table 2.

According to certain embodiments, the second target is a brain ECM protein. Such second targets include, but not limited to, for example Versican core protein (Chondroitin sulfate proteoglycan 2 or CSPG2), Tropoelastin (Elastin), Collagen alpha-2(IV) chain (Canstatin), Proteoglycan link protein 1 (Hyaluronan and proteoglycan link protein 1), Tenascin-R (TN-R), Proteoglycan link protein 2 (Hyaluronan and proteoglycan link protein 2), Collagen alpha-1(I) chain, Neurofilament-3 (NEF3), Immunoglobulin superfamily member 8 (IgSF8), Laminin subunit gamma-1 (LAMC1), Collagen alpha-1(VI) chain (Col6a1), and Collagen alpha-3(VI) chain (Col6a3), as shown in Table 2.

According to another embodiments, the second target is a brain ECM polysaccharide. Such second targets include but not limited to for example hyaluronic acid (HA) or hyaluronan.

In one embodiment, the first target is Transferrin receptor (TfR). An antigen-binding molecule comprising an antigen-binding domain binding to TfR is provided in the Examples. In particular, an anti-TfR/anti-MOG bispecific antibody is provided in Example 2 and 3 which exhibits advantageous pharmacokinetic properties. The results suggest that the combination of the brain transfer moiety (anti-TfR) and the brain retention moiety (anti-MOG) provides synergistic effect of longer lasting and high antibody concentration in brain, resulting in increased retention in brain and increased concentration in brain. Further, anti-TfR/anti-MOG bispecific antibodies and an anti-TfR/anti-CADM3 bispecific antibody are provided in Examples 4 and 5. It was surprisingly found that combination of the brain transfer moiety and the brain retention moiety in these bispecific antibodies showed equivalent or higher concentrations at day 1 compared with the antibodies with the brain transfer moiety alone, and much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain. Moreover, an anti-TfR/anti-CSPG5 bispecific antibody is provided in Examples 4 and 7. The results in Example 7 indicate that the combination of a brain transfer moiety binding to TfR and brain retention moiety binding to CSPG5 has superior potency in the transfer and retention into the brain.

In another embodiment herein, the first target is Basigin (CD147).

Bispecific antibodies comprising a brain transfer moiety binding to Basigin and a brain retention moiety binding to MOG (anti-Basigin/anti-MOG bispecific antibodies) are provided in Examples 4 and 5. It was surprisingly found that combination of the brain transfer moiety and the brain retention moiety in these bispecific antibodies showed equivalent or higher concentrations at day 1 compared with the antibodies with the brain transfer moiety alone, and much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain.

In another embodiment herein, the first target is Insulin receptor.

In another embodiment herein, the first target is insulin-like growth factor 1 receptor (IGF1R).

In another embodiment herein, the first target is Low density lipoprotein Receptor (LDLR).

In another embodiment herein, the first target is Low density lipoprotein receptor related protein (LRP). In preferred embodiments, LRP is LRP1.

In another embodiment herein, the first target is Diphtheria toxin Receptor.

In another embodiment herein, the first target is Glucose receptor. In preferred embodiments, Glucose receptor is Glut1.

In another embodiment herein, the first target is CD98hc.

In yet further embodiments, the second target is selected from the group consisting of Myelin Oligodendrocyte glycoprotein (MOG), Neuroglycan C/Chondroitin sulfate proteoglycan 5 (CSPG5), and Cell Adhesion Molecule 3 (CADM3).

In one embodiment, the second target is Myelin Oligodendrocyte glycoprotein (MOG). An antigen-binding molecule comprising an antigen-binding domain binding to MOG is provided in the Examples. In particular, an anti-TfR/anti-MOG bispecific antibody is provided in Example 2 and 3 which exhibits advantageous pharmacokinetic properties. The results suggest that the combination of the brain transfer moiety (anti-TfR) and brain retention moiety (anti-MOG) provides synergistic effect of longer lasting and high antibody concentration in brain, resulting in increased retention in brain and increased concentration in brain. Further, anti-TfR/anti-MOG bispecific antibodies as well as bispecific antibodies comprising a brain transfer moiety binding to Basigin and a brain retention moiety binding to MOG (anti-Basigin/anti-MOG bispecific antibodies) are provided in Examples 4, 5 and 7. It was surprisingly found that combination of the brain transfer moiety and the brain retention moiety in these bispecific antibodies showed equivalent or higher concentrations at day 1 compared with the antibodies with the brain transfer moiety alone, and much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain. Examples of other anti-MOG antigen-binding domains that can be used are described in e.g. WO2018123979. Examples of other anti-TfR antigen-binding domains that can be used are described in e.g. WO2012075037, WO2014033074 or Johnsen, Kasper Bendix, et al. “Targeting the transferrin receptor for brain drug delivery.” Progress in neurobiology 181 (2019): 101665.

In one embodiment, the second target is Neuroglycan C/Chondroitin sulfate proteoglycan 5 (CSPG5).

An anti-TfR/anti-CSPG5 bispecific antibody is provided in Examples 4 and 7. The results in Example 7 indicate that the combination of a brain transfer moiety binding to TfR and brain retention moiety binding to CSPG5 has superior potency in the transfer and retention into the brain. Examples of other anti-CSPG5 antigen-binding domains that can be used are described in e.g. WO2020004490. Examples of other anti-TfR antigen-binding domains that can be used are described in e.g. WO2012075037, WO2014033074 or Johnsen, Kasper Bendix, et al. “Targeting the transferrin receptor for brain drug delivery.” Progress in neurobiology 181 (2019): 101665.

In one embodiment, the second target is Cell Adhesion Molecule 3 (CADM3).

In the Examples, an anti-TfR/anti-CADM3 bispecific antibody is provided in Examples 4 and 5. It was surprisingly found that combination of the brain transfer moiety and the brain retention moiety in these bispecific antibodies showed equivalent or higher concentrations at day 1 compared with the antibodies with the brain transfer moiety alone, and much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain. Examples of other anti-CADM3 antigen-binding domains that can be used are described in e.g. WO2020004492. Examples of other anti-TfR antigen-binding domains that can be used are described in e.g. WO2012075037, WO2014033074 or Johnsen, Kasper Bendix, et al. “Targeting the transferrin receptor for brain drug delivery.” Progress in neurobiology 181 (2019): 101665.

In further embodiments, the first target is Transferrin receptor (TfR) and the second target is Myelin Oligodendrocyte glycoprotein (MOG).

In particular, an anti-TfR/anti-MOG bispecific antibody is provided in Example 2 and 3 which exhibits advantageous pharmacokinetic properties. The results suggest that the combination of the brain transfer moiety (anti-TfR) and brain retention moiety (anti-MOG) provides synergistic effect of longer lasting and high antibody concentration in brain, resulting in increased retention in brain and increased concentration in brain. Further, anti-TfR/anti-MOG bispecific antibodies are provided in Examples 4 and 5. It was surprisingly found that combination of the brain transfer moiety and the brain retention moiety in these bispecific antibodies showed equivalent or higher concentrations at day 1 compared with the antibodies with the brain transfer moiety alone, and much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain.

In further embodiments, the first target is Transferrin receptor (TfR) and the second target is Neuroglycan C/Chondroitin sulfate proteoglycan 5 (CSPG5).

An anti-TfR/anti-CSPG5 bispecific antibody is provided in Examples 4 and 7. The results in Example 7 indicate that the combination of a brain transfer moiety binding to TfR and brain retention moiety binding to CSPG5 has superior potency in the transfer and retention into the brain.

In further embodiments, the first target is Transferrin receptor (TfR) and the second target is Cell Adhesion Molecule 3 (CADM3).

In the Examples, an anti-TfR/anti-CADM3 bispecific antibody is provided in Examples 4 and 5. It was surprisingly found that combination of the brain transfer moiety and the brain retention moiety in these bispecific antibodies showed equivalent or higher concentrations at day 1 compared with the antibodies with the brain transfer moiety alone, and much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain.

In further embodiments, the first target is Basigin (CD147) and the second target is Myelin Oligodendrocyte glycoprotein (MOG).

Bispecific antibodies comprising a brain transfer moiety binding to Basigin and a brain retention moiety binding to MOG (anti-Basigin/anti-MOG bispecific antibodies) are provided in Examples 4 and 5. It was surprisingly found that combination of the brain transfer moiety and the brain retention moiety in these bispecific antibodies showed equivalent or higher concentrations at day 1 compared with the antibodies with the brain transfer moiety alone, and much higher concentrations at day 7 than the antibodies with the brain transfer moiety alone or the brain retention moiety alone. Also these further data suggest that the combination of the brain transfer moiety and the brain retention moiety has a synergistic effect on the potency of retention of an antibody in brain.

In further embodiments, the first target is Basigin (CD147) and the second target is Neuroglycan C/Chondroitin sulfate proteoglycan 5 (CSPG5).

In further embodiments, the first target is Basigin (CD147) and the second target is Cell Adhesion Molecule 3 (CADM3).

In further embodiments, the first target is Insulin receptor and the second target is Myelin Oligodendrocyte glycoprotein (MOG).

In further embodiments, the first target is Insulin receptor and the second target is Neuroglycan C/Chondroitin sulfate proteoglycan 5 (CSPG5).

In further embodiments, the first target is Insulin receptor and the second target is Cell Adhesion Molecule 3 (CADM3).

In further embodiments, the first target is glucose receptor and the second target is Myelin Oligodendrocyte glycoprotein (MOG).

In further embodiments, the first target is glucose receptor and the second target is Neuroglycan C/Chondroitin sulfate proteoglycan 5 (CSPG5).

In further embodiments, the first target is glucose receptor and the second target is Cell Adhesion Molecule 3 (CADM3).

In further embodiments, the first target is Low density lipoprotein Receptor (LDLR) and the second target is Myelin Oligodendrocyte glycoprotein (MOG).

In further embodiments, the first target is Low density lipoprotein Receptor (LDLR) and the second target is Neuroglycan C/Chondroitin sulfate proteoglycan 5 (CSPG5).

In further embodiments, the first target is Low density lipoprotein Receptor (LDLR) and the second target is Cell Adhesion Molecule 3 (CADM3).

In further embodiments, the first target is Low density lipoprotein receptor related protein (LRP) and the second target is Myelin Oligodendrocyte glycoprotein (MOG).

In further embodiments, the first target is Low density lipoprotein receptor related protein (LRP) and the second target is Neuroglycan C/Chondroitin sulfate proteoglycan 5 (CSPG5).

In further embodiments, the first target is Low density lipoprotein receptor related protein (LRP) and the second target is Cell Adhesion Molecule 3 (CADM3).

As described above, in aspects herein, the structure of the “second target” is not limited as long as the second target is expressed on the cell membrane of brain cells, or is a brain ECM protein or a brain ECM polysaccharide.

In embodiments, the “first target” is different from the “second target”. Accordingly, the “first antigen-binding domain” specifically binds a target which is different from the target to which the “second antigen-binding domain” specifically binds.

Accordingly, in certain embodiments, the antigen-binding molecule is bispecific and/or multispecific.

Moreover, in some embodiments, the second target is not a target selected from the group consisting of beta-secretase 1 (BACE1), Abeta, epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, TRK A, TRK B, TRK C, an alpha synuclein, a beta synuclein, a gamma synuclein, vascular endothelial growth factor (VEGF), neuropilin, a Semaphorin, Semaphorin 3A, Semaphorin 4A, Semaphorin 6A, myelin basic protein (MBP), MOG, PLP, MAG, aquaporin 4, glutamate receptor, and EpCAM.

In some embodiments, the second target is not beta-secretase 1 (BACE1). In some embodiments, the second target is not Abeta. In some embodiments, the second target is not epidermal growth factor receptor (EGFR). In some embodiments, the second target is not human epidermal growth factor receptor 2 (HER2). In some embodiments, the second target is not tau. In some embodiments, the second target is not apolipoprotein E (ApoE). In some embodiments, the second target is not alpha-synuclein. In some embodiments, the second target is not CD20. In some embodiments, the second target is not huntingtin. In some embodiments, the second target is not prion protein (PrP). In some embodiments, the second target is not leucine rich repeat kinase 2 (LRRK2). In some embodiments, the second target is not parkin, presenilin 1. In some embodiments, the second target is not presenilin 2. In some embodiments, the second target is not gamma secretase. In some embodiments, the second target is not death receptor 6 (DR6). In some embodiments, the second target is not amyloid precursor protein (APP). In some embodiments, the second target is not p75 neurotrophin receptor (p75NTR). In some embodiments, the second target is not caspase 6. In some embodiments, the second target is not TRK A. In some embodiments, the second target is not TRK B. In some embodiments, the second target is not TRK C. In some embodiments, the second target is not an alpha synuclein. In some embodiments, the second target is not a beta synuclein. In some embodiments, the second target is not a gamma synuclein. In some embodiments, the second target is not vascular endothelial growth factor (VEGF). In some embodiments, the second target is not neuropilin. In some embodiments, the second target is not a Semaphorin. In some embodiments, the second target is not Semaphorin 3A. In some embodiments, the second target is not Semaphorin 4A. In some embodiments, the second target is not Semaphorin 6A. In some embodiments, the second target is not myelin basic protein (MBP). In some embodiments, the second target is not MOG. In some embodiments, the second target is not PLP. In some embodiments, the second target is not MAG. In some embodiments, the second target is not aquaporin 4. In some embodiments, the second target is not glutamate receptor. In some embodiments, the second target is not EpCAM.

Embodiments of the Antigen-Binding Molecule Further Comprising at Least One Functional Moiety and/or at Least One In Vivo Half-Life Extension Moiety

In embodiments, the antigen-binding molecule of any of the aspects and embodiments herein further comprises at least one functional moiety and/or at least one in vivo half-life extension moiety. The antigen-binding molecule further comprising at least one functional moiety and/or at least one in vivo half-life extension moiety” may comprise the at least one functional moiety and/or at least one in vivo half-life extension moiety linked in different ways. Exemplary embodiments of antigen-binding molecules of the invention further comprising a functional moiety and an in vivo half-life extension moiety, or antigen-binding molecules of the invention further comprising a functional moiety (but not an in vivo half-life extension moiety), are shown in FIG. 2(a). In FIG. 2(a), one moiety is shown for each type of moiety (i.e. the first antigen-binding domain, the second antigen-binding domain, the functional moiety and/or the in vivo half-life extension moiety). However, it is also possible that two, three, four or more moieties are independently present for each type of moiety. Moreover, it is possible that the “first antigen-binding domain” and the “second antigen-binding moiety” are not linked directly, but are linked via an in vivo half-life extension moiety, such as an Fc region, or a functional moiety. Embodiments wherein the “first antigen-binding domain” and the “second antigen-binding moiety” are not linked directly, but are linked an Fc region are for example shown for some examples in FIGS. 2(b) and 2(c). FIGS. 2(b) and 2(c) show embodiments comprising an Fc region as exemplary in vivo half-life extension moiety.

One, two or more functional moieties may be linked to the antigen-binding molecule. For example, one or two functional moieties may be linked to one or both chains of an Fc region. For example, such one or two functional moieties may be a Fab, an scFv, an scFab or a VHH. Such embodiments are shown in FIG. 2(b). For example, two functional moieties may form a bivalent F(ab′)2 region linked to the Fc region. For example, the “first antigen-binding domain” and the “second antigen-binding moiety” may be linked to the Fc region. The “first antigen-binding domain” may be linked to the first chain of the Fc region and the “second antigen-binding moiety” may be linked to the second chain of the Fc region. Alternatively, the “first antigen-binding domain” and the “second antigen-binding moiety” may be linked to each other, for example as fusion protein, and may then by linked to one or both chains of the Fc region. In yet a further alternative exemplary embodiment, a “first antigen-binding domain” may be linked to a first light chain and a “second antigen-binding moiety” may be linked to a second light chain. In a further exemplary embodiment, the “first antigen-binding domain” and the “second antigen-binding moiety” may be linked to each other, for example as fusion protein, and may then by linked to one or two functional domains. In further examples, a “first antigen-binding domain” and/or the “second antigen-binding moiety” is linked to a functional moiety. For example, one functional moieties may be a Fab, an scFv, an scFab or a VHH and a “first antigen-binding domain” (or a “second antigen-binding moiety”) may be a Fab, an scFv, an scFab or a VHH. For example, such two monovalent Fabs, together forming a F(ab)2, may be linked to a Fc region, and a “second antigen-binding domain” (or a “first antigen-binding moiety”) may be linked to the “first antigen-binding domain” (or the “second antigen-binding moiety”). In yet further exemplary embodiments, one functional moieties may be a Fab, an scFv, an scFab or a VHH and a “first antigen-binding domain” (or a “second antigen-binding moiety”) may be a Fab, an scFv, an scFab or a VHH. For example, such two monovalent Fabs, together forming a F(ab)2, may be linked to a Fc region, and a “second antigen-binding domain” (or a “first antigen-binding moiety”) may be linked to the C-terminus of the Fc region.

### Embodiments of Domain(s) Arrangement

In certain embodiments of any of the aspects herein, the antigen-binding molecules comprise one or more antibody-type domains. For example, in the present Examples, a bispecific anti-TfR anti-MOG antibody was prepared. For example, it is possible to provide antigen-binding molecules comprising one or more variable regions or antibodies and antibody fragments comprising such one or more variable regions.

Accordingly, in embodiments of an antigen-binding molecule provided herein, the first antigen-binding domain is an antibody variable region comprising an antibody heavy chain variable region (VH) and an antibody light chain variable region (VL).

In particular embodiments, the first antigen-binding domain is a Fab, Fab′, F(ab′)₂, diabody, triabody, scFab, Fv, scFv, or single-domain antibody (VHH).

Further, in embodiments of an antigen-binding molecule provided herein, the second antigen-binding domain is an antibody variable region comprising an antibody heavy chain variable region (VH) and an antibody light chain variable region (VL).

In particular embodiments, the second antigen-binding domain is a Fab, Fab′, F(ab′)2, diabody, triabody, scFab, Fv, scFv, or single-domain antibody (VHH).

In embodiments of an antigen-binding molecule provided herein, the first antigen-binding domain is an antibody variable region comprising an antibody heavy chain variable region (VH) and an antibody light chain variable region (VL) and the second antigen-binding domain is an antibody variable region comprising an antibody heavy chain variable region (VH) and an antibody light chain variable region (VL).

In yet further embodiments, the first antigen-binding domain and the second antigen-binding domain are independently selected from the group consisting of an antibody variable region comprising an antibody heavy chain variable region (VH) and an antibody light chain variable region (VL), and a Fab, Fab′, F(ab′)₂, diabody, triabody, scFab, Fv, scFv, or single-domain antibody (VHH).

Moreover, it is possible that one first antigen-binding domain(s) is present or that two or more first antigen-binding domain(s) are present. Moreover, it is possible that one second antigen-binding domain(s) is present or that two or more second antigen-binding domain(s) are present.

In one embodiment, the antigen-binding molecule contains 1, 2, 3 or 4 first antigen-binding domain(s) and 1, 2 3 or 4 second antigen-binding domain(s).

Further, in one embodiment, the antigen-binding molecule further comprises at least one Fc region, such as one Fc region or two or more Fc regions. Exemplary embodiments of molecules comprising one Fc region are for example depicted in FIG. 2(b) and FIG. 2(c).

In yet further embodiments, the antigen-binding molecule contains 1, 2, 3 or 4 first antigen-binding domain(s) and 1, 2, 3 or 4 second antigen-binding domains and at least one Fc region.

In yet further preferred embodiments thereof, the first antigen-binding domain(s) and the second antigen-binding domain(s) are independently selected from the group consisting of an antibody variable region comprising an antibody heavy chain variable region (VH) and an antibody light chain variable region (VL), a Fab, Fab′, F(ab′)₂, diabody, triabody, scFab, Fv, scFv, and a single-domain antibody (VHH).

In a yet further embodiment, the antigen-binding molecule herein is a bispecific antibody. A plurality of embodiments of bispecific antibodies are shown in FIG. 2(b).

In one embodiment, the antigen-binding molecule comprises 1 first antigen-binding domain and 1 second antigen-binding domain.

For example, the Fab-arm exchange technology as described in WO2016/159213 can be used to generate bispecific antibodies.

### Further Embodiments for a Functional Moiety

In certain embodiments, the antigen-binding molecule of any of the aspects and embodiments herein further comprises at least one functional moiety.

In embodiments herein, the at least one functional moiety is an antibody or antigen-binding fragment thereof which specifically binds a membrane protein of i) an immune cell, especially wherein the immune cell is selected from the group consisting of a T cell, a killer cell, a helper T cell, a regulatory T cell, a B cell, a memory B cell, a NK cell, a NKT cell, a dendritic cell, a macrophage, an eosinophil, a neutrophil cell, and a basophil, ii) a tumor cell, or iii) an autoreactive cell.

In particular, embodiments, the antibody or antigen-binding fragment thereof specifically binds a membrane protein selected from the group consisting of a T cell receptor, CD3, CD137, CD40, CTLA4, a costimulatory molecule and a coinhibitory molecule.

For example, the antibody or antigen-binding fragment thereof may be selected from the group consisting of an antibody variable region comprising an antibody heavy chain variable region (VH) and an antibody light chain variable region (VL), a Fab, Fab′, F(ab′)₂, diabody, triabody, scFab, Fv, scFv, and a single-domain antibody (VHH). For example, the examples disclose bispecific antibodies comprising Fab or single chain Fab (scFab).

### Antibody Fragments

In certain embodiments, an antigen-binding molecule provided herein is an antibody which is an antibody fragment. Antibody fragments include, but are not limited to, single chain Fab (scFab), Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### Chimeric and Humanized Antibodies

In certain embodiments, an antigen-binding molecule provided herein is an antibody which is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); US Pat. Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain embodiments, an antigen-binding molecule provided herein is a human antibody or comprises a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB (registered trademark) technology; U.S. Pat. No. 7,041,870 describing K-M MOUSE (registered trademark) technology, and U.S. patent application Publication No. US 2007/0061900, describing V_{ELOCI}M_{OUSE} (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### Library-Derived Antibodies

Antigen-binding domains of an antigen-binding molecule provided herein, such as antibodies, may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### Multispecific Antibodies

In certain embodiments, an antigen-binding molecule provided herein is a multispecific antibody, e.g. a bispecific antibody. Multispecific antibodies are monoclonal antibodies that have binding specificities for at least two different sites. In certain embodiments, one of the binding specificities is for a first target that facilitates transfer of the antigen-binding domain into a mammalian brain and one is for a second target that is expressed on the cell membrane of brain cells. In another certain embodiments, one of the binding specificities is for a first target that facilitates transfer of the antigen-binding domain into a mammalian brain and one is for a second target that is a brain ECM protein or a brain ECM polysaccharide. In addition, one or more further binding specificities for other targets may be present. Multispecific antibodies comprising further specificities may also be used to localize cytotoxic agents to brain cells which express the second target. Bispecific and multispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

To efficiently obtain a bispecific antibody of interest comprising two heavy chain and two light chains, there are known amino acid substitutions and combinations in the CH1-CL domain interface that promote desired H chain-L chain association (such as e.g. described in WO2019065795) that can be used in an embodiment.

Engineered antibodies with three or more functional antigen binding sites, including “octopus antibodies,” are also included herein (see, e.g. US 2006/0025576A1).

The antibody or fragment herein also includes a “Dual Acting Fab” or “DAF” comprising an antigen binding site that binds to a first target as well as to the second target (see, US 2008/0069820, for example).

### Antigen-Binding Molecule Variants and in Particular Antibody Variants

In certain embodiments, amino acid sequence variants of the antigen-binding molecules and in particular are antibodies provided herein are contemplated. For example, it may be desirable to improve the binding affinity and/or other biological properties of the antigen-binding molecule and in particular antibody. Amino acid sequence variants of antigen-binding molecule and in particular antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antigen-binding molecule and in particular antibody. Any combination of deletion, insertion, and substitution can be made to arrive at the final construct, provided that the final construct possesses the desired characteristics, e.g., antigen-binding.

### Glycosylation Variants

In certain embodiments, an antigen-binding molecules and in particular antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antigen-binding molecules and in particular antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antigen-binding molecules and in particular antibody variants with certain improved properties.

In one embodiment, antigen-binding molecules and in particular antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antigen-binding molecule variants and in particular antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In one preferred embodiment of an antigen-binding molecule provided herein, at least one first antigen-binding domain and at least one second antigen-binding domain are linked to an Fc region.

### Fc Region Variants

In certain embodiments, one or more amino acid modifications may be introduced into the Fc region of an antigen-binding molecules and in particular an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In one preferred embodiment of an antigen-binding molecule provided herein, 1 first antigen-binding domain and 1 second antigen-binding domain are linked to an Fc region.

In one preferred embodiment of an antigen-binding molecule provided herein, the Fc region is a Fc region of which the ability to bind to the activating Fc gamma receptor is decreased compared to the ability of an Fc region of a native human IgG to bind to the activating Fc gamma receptor.

In one preferred embodiment, the activating Fc gamma receptor is human Fc gamma RIa, human Fc gamma RIIa(R), human Fc gamma RIIa(H), human Fc gamma RIIIa(V), or human Fc gamma RIIIa(F).

In one preferred embodiment, the activating Fc gamma receptor is human Fc gamma RIa. In one preferred embodiment, the activating Fc gamma receptor is human Fc gamma RIIa(R). In one preferred embodiment, the activating Fc gamma receptor is human Fc gamma RIIa(H). In one preferred embodiment, the activating Fc gamma receptor is human Fc gamma RIIIa(V). In one preferred embodiment, the activating Fc gamma receptor is human Fc gamma RIIIa(F).

In one preferred embodiment, the Fc region comprises one or more of the following amino acid substitutions (all positions by EU numbering):
Ala, Arg, Asn, Asp, Gln, Glu, Gly, His, Lys, Met, Phe, Pro, Ser, Thr, or Trp at position 234;
Ala, Asn, Asp, Gln, Glu, Gly, His, Ile, Lys, Met, Pro, Ser, Thr, Val, or Arg at position 235;
Arg, Asn, Gln, His, Leu, Lys, Met, Phe, Pro, or Tyr at position 236; Ala, Asn, Asp, Gln, Glu, His, Ile, Leu, Lys, Met, Pro, Ser, Thr, Val, Tyr, or Arg at position 237;
Ala, Asn, Gln, Glu, Gly, His, Ile, Lys, Thr, Trp, or Arg at position 238; Gln, His, Lys, Phe, Pro, Trp, Tyr, or Arg at position 239;
Ala, Arg, Asn, Gln, Gly, His, Ile, Leu, Lys, Met, Phe, Ser, Thr, Trp, Tyr, or Val at position 265;
Ala, Arg, Asn, Asp, Gln, Glu, Gly, His, Lys, Phe, Pro, Ser, Thr, Trp, or Tyr at position 266;
Arg, His, Lys, Phe, Pro, Trp, or Tyr at position 267;
Ala, Arg, Asn, Gln, Gly, His, Ile, Leu, Lys, Met, Phe, Pro, Ser, Thr, Trp, Tyr, or Val at position 269;
Ala, Arg, Asn, Gln, Gly, His, Ile, Leu, Lys, Met, Phe, Pro, Ser, Thr, Trp, Tyr, or Val at position 270;
Arg, His, Phe, Ser, Thr, Trp, or Tyr at position 271;
Arg, Asn, Asp, Gly, His, Phe, Ser, Trp, or Tyr at position 295;
Arg, Gly, Lys, or Pro at position 296;
Ala at position 297;
Arg, Gly, Lys, Pro, Trp, or Tyr at position 298;
Arg, Lys, or Pro at position 300;
Lys or Pro at position 324;
Ala, Arg, Gly, His, Ile, Lys, Phe, Pro, Thr, Trp, Tyr, or Val at position 325;
Arg, Gln, His, Ile, Leu, Lys, Met, Phe, Pro, Ser, Thr, Trp, Tyr, or Val at position 327;
Arg, Asn, Gly, His, Lys, or Pro at position 328;
Asn, Asp, Gln, Glu, Gly, His, Ile, Leu, Lys, Met, Phe, Ser, Thr, Trp, Tyr, Val, or Arg at position 329;
Pro or Ser at position 330;
Arg, Gly, or Lys at position 331;
Arg, Lys, or Pro at position 332.

In one preferred embodiment, the Fe region comprises between 1 and 10, between 1 and 5, between 1 and 4, between 1 and 3, or 1, 2, 3, 4, or 5 substitutions in the Fe region.

In certain other embodiments, the invention contemplates an antigen-binding molecules and in particular antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1™ non-radioactive cytotoxicity assay for flow cytometry (CellTechnology, Inc. Mountain View, CA; and CytoTox 96 (registered trademark) non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antigen-binding molecules and in particular antibodies comprising an Fc region with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antigen-binding molecules and in particular antibodies antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antigen-binding molecule and in particular antibody variant comprises an Fc region with one or more amino acid substitutions which improve ADCC, e.g., substitutions at positions 298, 333, and/or 334 of the Fe region (EU numbering of residues).

In some embodiments, alterations are made in the Fe region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antigen-binding molecules and in particular antibodies with increased half-lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### Cysteine Engineered Antibody Variants

In certain embodiments, it may be desirable to create cysteine engineered antigen-binding molecules, in particular antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### Antibody Derivatives

In certain embodiments, an antigen-binding molecule and in particular antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antigen-binding molecule and in particular antibody to be improved, whether the antibody derivative will be used in a therapy under defined conditions, etc.

In another embodiment, conjugates of an antigen-binding molecule and in particular antibody and non-proteinaceous moiety that may be selectively heated by exposure to radiation are provided. In one embodiment, the non-proteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

### Recombinant Methods and Compositions

In an aspect herein, a nucleic acid or two or more nucleic acids encoding the antigen-binding molecule according to any of the embodiments herein is provided. Optionally the nucleic acid or two or more nucleic acids are operably linked to a promotor. Further provided is a vector or two or more vectors comprising the nucleic acid or two or more nucleic acids provided herein. Further provided herein is a host cell comprising (i) the nucleic acid or two or more nucleic acids provided herein; (ii) the vector or two or more vectors provided herein; and/or (iii) capable of expressing antigen-binding molecule according to any one of the aspects and embodiments herein.

Further provided is a method of producing an antigen-binding molecule provided herein, comprising culturing the host cell provided herein so that the antibody is produced; optionally further comprising recovering the antibody from the host cell.

Antigen-binding molecule and in particular antibodies may be produced using recombinant methods and compositions, e.g., as described in U.S. Pat. No. 4,816,567. In one embodiment, an isolated nucleic acid encoding a first antigen-binding domain comprising a variable region described herein is provided. Such nucleic acid may encode an amino acid sequence comprising the VL and/or an amino acid sequence comprising the VH of the first antigen-binding domain comprising a variable region (e.g., the light and/or heavy chains of the antibody), and optionally further encoding an amino acid sequence comprising the VL and/or an amino acid sequence comprising the VH of the second antigen-binding domain comprising a variable region (e.g., the light and/or heavy chains of the antibody). Alternatively, the sequences may be provided as two or more separate nucleic acids. In a further embodiment, one or more vectors (e.g., expression vectors) comprising such nucleic acid(s) are provided. In a further embodiment, a host cell comprising such nucleic acid(s) is provided. In one such embodiment, a host cell comprises for example (e.g., has been transformed with): (1) a vector comprising a nucleic acid that encodes an amino acid sequence comprising the VL of the first antigen-binding domain and an amino acid sequence comprising the VH of the first antigen-binding domain, and a nucleic acid that encodes an amino acid sequence comprising the VL of the second antigen-binding domain and an amino acid sequence comprising the VH of the second antigen-binding domain or (2) a first vector comprising a nucleic acid that encodes an amino acid sequence comprising the VL of the first antigen-binding domain and a second vector comprising a nucleic acid that encodes an amino acid sequence comprising the VH of the first antigen-binding domain and a third vector comprising a nucleic acid that encodes an amino acid sequence comprising the VL of the second antigen-binding domain and a fourth vector comprising a nucleic acid that encodes an amino acid sequence comprising the VH of the second antigen-binding domain. In one embodiment, the host cell is eukaryotic, e.g. a Chinese Hamster Ovary (CHO) cell or lymphoid cell (e.g., Y0, NS0, Sp2/0 cell). In one embodiment, a method of making an antigen-binding molecule, and in particular antibody is provided, wherein the method comprises culturing a host cell comprising a nucleic acid encoding the antigen-binding molecule, in particular antibody, as provided above, under conditions suitable for expression of the antibody, and optionally recovering the antigen-binding molecule antibody from the host cell (or host cell culture medium).

For recombinant production of an antigen-binding molecules, in particular an antibody, nucleic acid encoding an antigen-binding molecules, such as an antibody, e.g., as described above, is isolated and inserted into one or more vectors for further cloning and/or expression in a host cell. Such nucleic acid(s) may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.). After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Methods of Production and Screening

Antigen-binding molecules provided herein may be identified, screened for, or characterized for their physical/chemical properties and/or biological activities by various assays known in the art.

Further provided herein is a method for producing an antigen-binding molecule, which comprises the steps of:
(a) selecting a first antigen-binding domain that specifically binds a first target that facilitates transfer of the antigen-binding molecule into a mammalian brain;
(b) selecting a second antigen-binding domain that specifically binds a second target wherein the second target (i) is expressed on the cell membrane of brain cells or (ii) is a brain ECM protein or a brain ECM polysaccharide;
(c) obtaining one or more nucleic acid(s) encoding an antigen-binding molecule in which an antigen-binding domain and an antigen-binding domain prepared in (a) and (b) are linked; and
(d) producing an antigen-binding molecule using the one or more nucleic acid(s) prepared in (c).

In a preferred embodiment herein, the method further comprising step (e):
(e) determining that the (i) retention (ii) concentration or (iii) exposure of said antigen-binding molecule according to (d) in the brain of a subject is increased compared to a control antigen-binding molecule, wherein the control antigen-binding molecule differs from the antigen-binding molecule according to (d) only in that it comprises either one but not both of the antigen-binding domains defined in (a) and (b).

The embodiments of any other aspects and embodiments herein also apply to these methods.

Further provided is a method for screening an antigen-binding molecule, which comprises the steps of:
(a) selecting a first antigen-binding domain that specifically binds a first target that facilitates transfer of the antigen-binding molecule into a mammalian brain;
(b) selecting a second antigen-binding domain that specifically binds a second target wherein the second target (i) is expressed on the cell membrane of brain cells or (ii) is a brain ECM protein or a brain ECM polysaccharide;
(c) obtaining one or more nucleic acid(s) encoding an antigen-binding molecule in which an antigen-binding domain and an antigen-binding domain prepared in (a) and (b) are linked; and
(d) producing an antigen-binding molecule using the one or more nucleic acid(s) prepared in (c).

In a preferred embodiment herein, the method further comprising step (e):
(e) determining that the (i) retention (ii) concentration or (iii) exposure of said antigen-binding molecule according to (d) in the brain of a subject is increased compared to a control antigen-binding molecule, wherein the control antigen-binding molecule differs from the antigen-binding molecule according to (d) only in that it comprises either one but not both of the antigen-binding domains defined in (a) and (b).

The embodiments of any other aspects and embodiments herein also apply to these methods.

In one embodiment, an antigen-binding molecule such as an antibody of the invention is tested for its antigen binding activity, e.g., by known methods such as ELISA, Western blot, etc.

### Methods and Compositions for Diagnostics and Detection

In certain embodiments, any of the antigen-binding molecules provided herein is useful for detecting the presence of the first target and/or second target in a biological sample. The term “detecting” as used herein encompasses quantitative or qualitative detection. In certain embodiments, a biological sample comprises a cell or tissue, such as brain tissue and brain cells.

In certain embodiments, labeled antigen-binding molecules, in particular antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### Pharmaceutical Formulations

Provided herein is a pharmaceutical composition comprising the antigen-binding molecule according to any one of the aspects and embodiments herein and one or more pharmaceutically acceptable carrier(s) or excipient(s). Further, a pharmaceutical composition is provided comprising (i) an antigen-binding molecule provided herein, (ii) a nucleic acid or two or more nucleic acids encoding the antigen-binding molecule provided herein, (iii) a vector or two or more vectors provided herein, or (iv) a host cell provided herein, and one or more pharmaceutically acceptable carrier(s) or excipient(s).

Pharmaceutical formulations of an antigen-binding molecule as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

Any of the antigen-binding molecules and in particular antibodies provided herein, as well as nucleic, vectors or host cells and pharmaceutical compositions may be used in therapeutic methods.

In one embodiment, an antigen-binding molecule antibody for use as a medicament is provided.

In an embodiment, (i) an antigen-binding molecule provided herein, (ii) a nucleic acid or two or more nucleic acids encoding the antigen-binding molecule provided herein, (iii) a vector or two or more vectors provided herein, or (iv) a host cell provided herein, or (iv) a pharmaceutical composition provided herewith, for use as a medicament is provided herewith.

In an embodiment, (i) an antigen-binding molecule provided herein, (ii) a nucleic acid or two or more nucleic acids encoding the antigen-binding molecule provided herein, (iii) a vector or two or more vectors provided herein, or (iv) a host cell provided herein, or (iv) a pharmaceutical composition provided herewith, for use as a diagnostic is provided herewith.

In an embodiment, provided herewith is (i) an antigen-binding molecule provided herein, (ii) a nucleic acid or two or more nucleic acids encoding the antigen-binding molecule provided herein, (iii) a vector or two or more vectors provided herein, or (iv) a host cell provided herein, or (iv) a pharmaceutical composition provided herewith, for use in the treatment and/or prevention of a brain disorder or disease in a subject.

In an embodiment, provided herewith is a method of treating and/or preventing a brain disorder or disease in a subject in need thereof, the method comprising administering to said subject a therapeutically effective amount of (i) an antigen-binding molecule provided herein, (ii) a nucleic acid or two or more nucleic acids encoding the antigen-binding molecule provided herein, (iii) a vector or two or more vectors provided herein, or (iv) a host cell provided herein, or (iv) a pharmaceutical composition provided herewith.

In an embodiment, provided herewith is (i) an antigen-binding molecule provided herein, (ii) a nucleic acid or two or more nucleic acids encoding the antigen-binding molecule provided herein, (iii) a vector or two or more vectors provided herein, or (iv) a host cell provided herein, or (iv) a pharmaceutical composition provided herewith for use in the preparation of a medicament for the treatment and/or prevention of a brain disorder or disease.

In an embodiment, provided herewith is (i) an antigen-binding molecule provided herein, (ii) a nucleic acid or two or more nucleic acids encoding the antigen-binding molecule provided herein, (iii) a vector or two or more vectors provided herein, or (iv) a host cell provided herein, or (iv) a pharmaceutical composition provided herewith, for the preparation of a medicament for the treatment and/or prevention of a brain disorder or disease.

In some embodiments, the brain disorder or diseases is selected from the group consisting of neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heredodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Hallervorden-Spatz syndrome, Lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), psychiatric disorders, cancer (e.g. of the CNS, including brain metastases resulting from cancer elsewhere in the body). In some embodiments, the brain disorder or diseases is selected from the group consisting of Alzheimer's disease, Pompe disease, Frontotemporal dementia (FTD), and Amyotrophic lateral sclerosis (ALS)).

In an embodiment, an antigen-binding molecule of any of the aspects or embodiments herein is provided, for use (i) in a method of increasing the concentration of the antigen-binding molecule in the brain of a subject in need thereof; (ii) in a method of increasing exposure of the antigen-binding molecule in the brain of a subject in need thereof; and/or (iii) for the retention of the antigen-binding molecule in the brain of a subject in need thereof; optionally wherein the subject is a human.

In one embodiment, a “subject” is a human.

In one embodiment, the “subject in need thereof” is a subject suffering from or suspected to suffer from a brain disease or disorder.

Antigen-binding molecules, in particular antibodies of the invention can be used either alone or in combination with other agents in a therapy. For instance, an antibody of the invention may be co-administered with at least one additional therapeutic agent. The choice of the additional therapeutic agent will depend on the brain disease or disorder to be treated.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the antigen-binding molecule and administration of an additional therapeutic agent occur within about one month, or within about one, two or three weeks, or within about one, two, three, four, five, or six days, of each other. Antigen-binding molecules of the invention can also be used in combination with radiation therapy.

An antigen-binding molecule (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antigen-binding molecules of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antigen-binding molecule need not be, but is optionally formulated with one or more agents currently used to prevent or treat the disorder in question. The effective amount of such other agents depends on the amount of antigen-binding molecule present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antigen-binding molecule of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 micro g/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the antigen-binding molecule would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. The progress of this therapy is easily monitored by conventional techniques and assays.

### Brain Disorder or Diseases

A “neurological disorder” as used herein refers to a disease or disorder which affects the brain/CNS and/or which has an etiology in the brain/CNS. Exemplary brain diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of brain disorder or diseases include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heredodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Hallervorden-Spatz syndrome, Lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), psychiatric disorders, cancer (e.g. of the CNS, including brain metastases resulting from cancer elsewhere in the body). In some specific examples, the brain disorder or diseases is selected from the group consisting of Alzheimer's disease, Pompe disease, Frontotemporal dementia (FTD), and Amyotrophic lateral sclerosis (ALS).

In some embodiments, the corresponding functional moiety comprised in the antigen-binding molecule of the invention are described herein and include, but are not limited to: antibodies, aptamers, proteins, peptides, inhibitory nucleic acids and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of brain disorder or diseases they may be used to treat and the corresponding functional moieties comprised in the antigen-binding molecule of the invention are described herein are provided in the following Table A:

TABLE A
Non-limiting examples of functional moieties and the corresponding
brain disorders or diseases they may be used to treat.

| | |
|---|---|
| Progranuline | Frontotemporal dementia (FTD), Amyotrophic |
| | lateral sclerosis (ALS) |
| Acid alpha-glucosidase (GAA) | Pompe discase |
| Neprilysin | Alzheimer's disease |
| Anti-SNCA (alpha-Synuclein) Antibody | Lewy body dementia, Parkinson's disease, |
| | Multiple system atrophy, Synucleinopathics |
| Anti-C1Q (Complement C1q Subcomponent) | Neurodegeneration |
| Antibody | |
| Anti-CALCA (Calcitonin Gene-Related | Cluster headache, Fibromyalgia, Migraine, Post- |
| Peptide 1; CALC1) Antibody | traumatic headache |
| Anti-CALCRL (Calcitonin Gene-Related | Migraine prophylaxis |
| Peptide Receptor; CGRPR) Antibody | |
| Anti-IL6R (Interleukin-6 Receptor | Human autoimmune encephalitis (HAE), |
| Subunit alpha; CD126) Antibody | MOG antibody-associated diseases, |
| | Myasthenia gravis |
| | Neuromyelitis optica |
| Anti-CD40LG (CD40 Ligand; CD154) | Amyotrophic lateral sclerosis, |
| Antibody | Autoimmune disease |
| Anti-CD19 Antibody | Autoimmune disease, Multiple sclerosis, |
| | relapsing-remitting, Myasthenia gravis, |
| | Neuromyelitis optica |
| Anti-CD20 Antibody | Multiple sclerosis |
| | Multiple sclerosis, primary progressive |
| | Multiple sclerosis, relapsing-remitting |
| | Systemic lupus erythematosus |
| | Neuromyelitis optica |
| Anti-CD49d (VLA-4) Antibody | Autoimmune disease |
| | Crohn's disease |
| | Graft-versus-host disease |
| | Multiple myeloma |
| | Multiple sclerosis |
| | Multiple sclerosis, relapsing-remitting |
| | Multiple sclerosis, secondary progressive |
| | Clinically isolated syndrome (CIS) |
| | Crohn's disease |
| | Seizures |
| | Stroke, ischemic |
| Anti-CD52 (CAMPATH-1 Antigen) Antibody | Multiple sclerosis |
| | Multiple sclerosis, relapsing-remitting |
| Anti-GJA1 (Cx43, Connexin 43, Gap | Injury, spinal cord |
| Junction alpha-1 Protein) Antibody | Stroke |
| Anti-HERVW-1 (Syncytin; Endogenous | Amyotrophic lateral sclerosis |
| Retrovirus Group W Member 1) Antibody | Chronic inflammatory demyelinating |
| | polyneuropathy |
| | Cognitive disorders |
| | Diabetes type 1 |
| | Fatigue |
| | Multiple sclerosis |
| | Multiple sclerosis, relapsing-remitting |
| | Post COVID-19 condition |
| Anti-EPHA4 (Ephrin Type-A Receptor 4) | Dementia, Alzheimer type |
| Antibody | |
| Anti-FAM19A5 (Chemokine-Like Protein | Neurodegeneration |
| TAFA-5) Antibody | |
| Anti-LGALS3 (Galectin-3) Antibody | Dementia, Alzheimer type |
| | Stroke, ischemic |
| Anti-IL1A (Interleukin-1alpha) Antibody | Stroke, ischemic |
| Anti-beta-Nerve Growth Factor (NGF) | Diabetic neuropathy |
| Drugs Targeting Tumor Necrosis Factor | Pain |
| (TNF) Receptors Antibody | |
| Anti-PACAP (Pituitary Adenylate Cyclase- | Headache |
| Activating Polypeptide; ADCYAP1) Antibody | Migraine |
| Anti-CD274 (PD-L1) Antibody | Dementia, Alzheimer type |
| Anti-Repulsive Guidance Molecule A (RGM | HTLV-1-associated myelopathy-tropical |
| Domain Family Member A) Antibody | spastic paraparesis |
| | Injury, spinal cord |
| | Multiple sclerosis, Stroke, ischemic |
| Anti-SEMA4D (Semaphorin 4D) Antibody | Dementia, Alzheimer type |
| | Huntington's disease |
| | Multiple sclerosis |
| Anti-SOD1 (Superoxide Dismutase) Antibody | Amyotrophic lateral sclerosis |
| Anti-MAPT (Microtubule-Associated Protein | Alzheimer disease, early onset |
| Tau; PHF-tau) Antibody | Dementia, Alzheimer type |
| Anti-TREM2 (Triggering Receptor Expressed | Dementia, Alzheimer type |
| on Myeloid Cells 2) Antibody | Neurodegeneration |

### Articles of Manufacture

In another aspect of the invention, an article of manufacture containing materials useful for the treatment, prevention and/or diagnosis of the brain disorder or disease described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is an antigen-binding molecule, in such as an antibody, of the invention. The label or package insert indicates that the composition is used for treating the condition of choice. Moreover, the article of manufacture may comprise (a) a first container with a composition contained therein, wherein the composition comprises an antigen-binding molecule of the invention; and (b) a second container with a composition contained therein, wherein the composition comprises a cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### Example 1. Concept of Improvement of an Antigen-Binding Molecule, Such as an Antibody, Concentration and Exposure in Brain by Designing a Molecule Having a Brain Transfer Moiety and a Brain Retention/Targeting Moiety

One of the major limitations of therapeutics in CNS area is known to be low exposure of therapeutics due to low transfer efficacy. To solve this issue blood-brain-barrier (BBB) crossing technologies using Receptor Mediated Transfer system (RMT) have been developed and used for therapeutics. One of the examples of RMT is utilizing transferrin receptor binding moiety such as antibodies that bind to the transferrin receptor (TfR), which is expressed in endothelial cells, including those of the BBB and allows for transport across the BBB by RMT [Lajoie, Jason M., and Eric V. Shusta. “Targeting receptor-mediated transport for delivery of biologics across the blood-brain barrier.” Annual review of pharmacology and toxicology 55 (2015): 613].

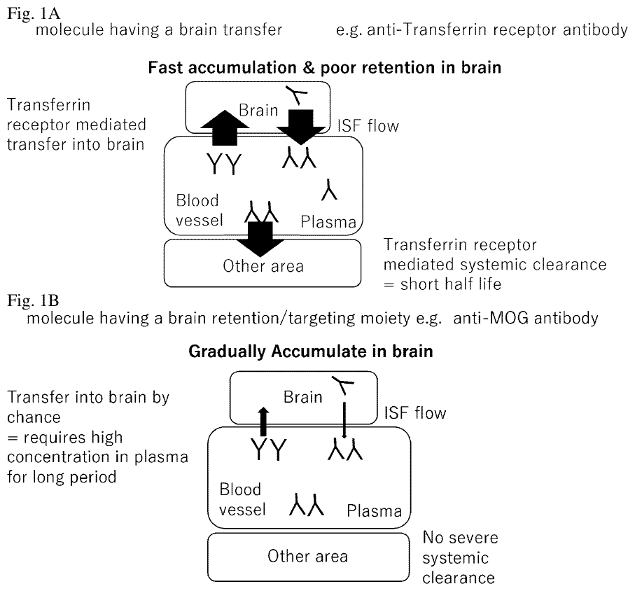

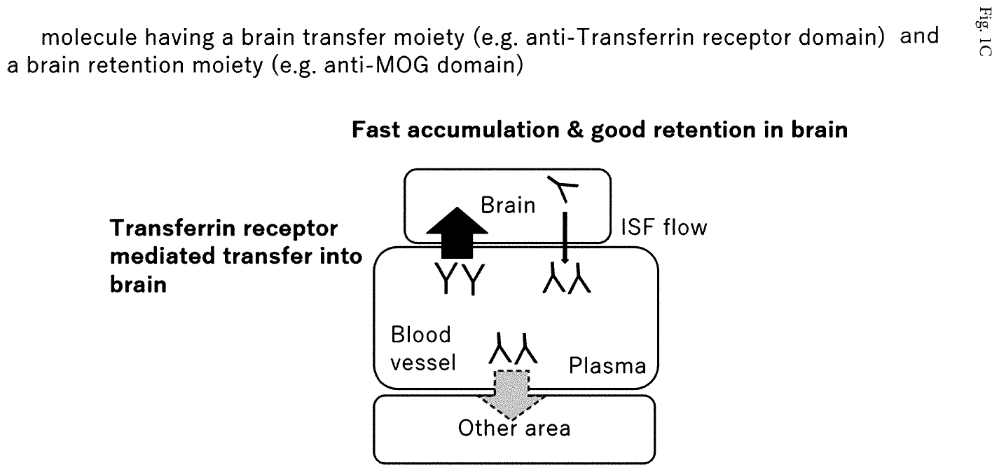

Additionally, other receptors, such as the insulin receptor, low density lipoprotein receptor (LDLR) are also RMT systems reported that can be used as RMT-based BBB-transfer moiety to deliver biologics to the brain [Lajoie, Jason M., and Eric V. Shusta, supra]. RMT systems such as anti-TfR antibody and anti-insulin receptor antibody are known to have high brain transfer ratio compared with that of IgG in plasma. However, it is well known that anti-transferrin receptor antibody has short half-life in plasma due to expression of transferrin receptor not only in the vascular endothelial cells of the brain, but also systemically (i.e. broad expression throughout the body such as in many other non-brain cell types). Consequently, anti-TfR antibody also delivers drugs to tissues other than the brain, resulting in a short half-life in the blood. Similarly, other antibody having BBB transfer ability showed fast clearance and short half-life (anti-CD98hc antibody, Neuron Vol 90, Issue 1, 2016, 70-82). In addition, since there is a regular flow turnover of interstitial fluid (ISF) within brain, the antibody transferred into brain will be washed away from brain to blood circulation rapidly. Therefore, after flowing to blood circulation, the antibody needs to be transferred back to brain again through BBB. For this reason, therapeutic molecules fused with anti-transferrin receptor antibody showed improvement of antibody concentration in brain only sustain for a short or limited duration, which limits the effectiveness of said therapeutics molecules in brain (FIG. 1(a)).

A different approach has been proposed and achieved high antibody concentration and retention in brain by using an antibody that binds to myelin oligodendrocyte glycoprotein (MOG, which is a brain specific antigen) (PLoS One 2019, Apr. 12; 14(4)e0214404), which slows down the export of the antibodies from the brain as a result of binding to MOG.

However, the inventors of the present invention envisioned, without being bound to the theory, that the mechanism of this “brain-retention” approach, whilst being able to overcome the short exposure of molecule having transferrin receptor binding domain, requires that anti-MOG antibodies to have long half-life in plasma in order to be gradually accumulated and achieve high concentration in brain because it requires antibodies to be continuously supplied from blood circulation into the brain (FIG. 1(b)). For this reason anti-MOG antibodies require sustained high concentration in plasma (or long half-life in plasma) for achieving and maintaining high concentration in brain and consequently, its brain-retention effect will be reduced or limited when it is combined with a therapeutic molecule which has inherent short half-life in plasma. Another limitation of this approach is that anti-MOG antibody requires considerable long time to gradually accumulate and reach the desirable antibody concentration and exposure in brain after dosing, which is not ideal in terms of PK profile.

Considering this situation new technology which enables molecule to have both high antibody concentration and exposure in brain rapidly and lasts longer period is desired.

As explained above, given the BBB-crossing technology such as anti-TfR antibody is known to have short half-life in plasma, whereas brain-retention technology such as anti-MOG antibody is expected to require anti-MOG antibody to have long half-life in plasma in order to be gradually accumulated and achieve high concentration in brain, attempt to combine both technologies may not appear to be a promising approach, and hence for this reason a molecule having both anti-TfR domain and anti-MOG domain has not been reported or tested.

In the present application, the inventors surprisingly found that a molecule which comprises both a brain transfer moiety and a brain retention moiety, despite having a short half-life in plasma, is able to achieve both high antibody concentration and exposure in brain rapidly and high retention for long period in brain (FIG. 1(c)). FIG. 2(a) is a schematic drawing showing the concept of exemplary embodiments of the antigen-binding molecules of the present inventions, which comprise (1) a brain transfer moiety, (2) a brain retention moiety, optionally (3) a functional moiety, and, further optionally, in addition, a half-life extension moiety.

The brain transfer moiety is an antigen-binding domain which specifically binds a molecule that facilitates penetration or transfer of the antigen-binding molecule into brain. For example, said molecule that facilitates penetration of transfer into brain is a receptor expressed on vascular endothelial cells, preferably vascular endothelial cells of the blood-brain barrier (BBB), which allows transferring of biologics to the brain via RMT route. Transferring of biologics to brain via RMT-based receptors by using e.g. anti-TfR antibody, anti-insulin receptor antibody or anti-LDLR antibody have been well-studied and reported [Lajoie, Jason M., and Eric V. Shusta, supra]. In one non-limiting example, said molecule is a molecule that specifically expressed on vascular endothelial cells of the blood-brain barrier (BBB). Exemplary molecule is one or more molecule which is selected from those listed in Table 1. In one example, the brain transfer moiety is an antigen-binding domain which specifically binds Transferrin receptor (TfR). In another example, the brain transfer moiety is an antigen-binding domain which specifically binds Insulin receptor.

In one embodiment, the brain-retention moiety is an antigen-binding domain which binds a molecule that is expressed on the cell membrane of brain cells, or is a brain ECM protein or a brain ECM polysaccharide. It has been reported that binding of brain antigen can slow down the efflux of the molecule from the brain, thereby achieving higher retention in brain (Nakano, Ryosuke, et al. “A new technology for increasing therapeutic protein levels in the brain over extended periods.” Plos one 14.4 (2019): e0214404; WO2018123979, WO2020004490, WO2020004492). An exemplary target is/are one or more target(s) selected from those listed in Table 2.

In some specific embodiments, by selecting an antigen that is brain-specific, it allows the antibody concentration is increased specifically only in the brain, and maintain over an extended period in brain. In one example of the present disclosure, said brain cells can be any brain cell, such as a brain cell selected from the group consisting of oligodendrocytes, astrocytes, neurons, microglia. Exemplary target molecules and cells to be bound by the brain retention moiety are shown in Table 2. In addition, said target molecule can be any molecule that is determined to be specifically expressed on a brain-specific cell by characterization of its expression pattern as follows; 1. Select the molecule of interest, 2. Check the expression level (either RNA/Protein/actual data) 3. Compare the expression level in brain and other area (organ/tissue), 4. Pick up the molecule of interest which has higher expression level in brain from other area (organ/tissue). Cell markers which usually used for FACS analysis or other analysis to identify the cell types are also useful for brain retention moiety.

The functional moiety can be any molecule that has therapeutic function such as agonist, antagonist, enzyme, modulator, stabilizer, cell death inducer and any molecular format such as nucleic acid, small molecule, cyclic peptide, peptide, ligand, cytokine, chemokine, growth factor, enzyme and antigen binding domain and so on. Optionally, one or more half-life extension moiety can be fused to the molecule as well. The examples of the half-life extension moiety are immunoglobulin's Fc region, albumin binding domain, FcRn binding protein, FcRn binding peptide and PEG.

FIGS. 2(b) and (c) are schematic drawings showing exemplary molecular formats of the antigen-binding molecules of the present disclosure. In one example, the antigen-binding molecules comprise a first Fab region that binds a molecule that facilitates penetration or transfer of the antigen-binding molecule into brain (i.e. brain-transfer moiety) and a second Fab regions that binds a molecule that is specifically expressed on a brain-specific cell (i.e. brain-retention moiety), and optionally further comprises a functional moiety. The structure of said brain retention moiety and brain transfer moiety is not limited to Fab region, but can also in the form of antibody fragments such as single chain Fab (scFab), Fv, Fab, Fab′, F(ab′)₂, diabody, triabody, scFv, VHH, diabodies, or F(ab′)2 fragments, or a non-antibody binder (e.g. affibody, DARPins, FN3, aptamer, anticalins). In the examples, bispecific antibodies comprising single chain Fab (scFab) or Fab were prepared.

TABLE 1
Targets of brain transfer moiety (first target that facilitates
transfer of the antigen-binding molecule into a mammalian brain)

| No | Target receptor/protein |
|---|---|
| 1 | Transferrin receptor (TfR) |
| 2 | Basigin(CD147) |
| 3 | Glut1 |
| 4 | LRP1 |
| 5 | Ldlrad3 |
| 6 | CD320 |
| 7 | Insulin receptor |
| 8 | Low density lipoprotein |
| | Receptor (LDLR) |
| 9 | Low density lipoprotein |
| | receptor related protein (LRP) |
| 10 | Diphtheria toxin Receptor |
| 11 | Glucose receptor |
| 12 | CD98hc |
| 13 | TMEM30A |
| 14 | Leptin receptor (LepR) |
| 15 | heparan sulfate chains branching |
| | from proteoglycan |
| | (HSPG) |

TABLE 2
Target molecules (second target that is expressed on the cell membrane of
brain cells, or is a brain ECM protein or a brain ECM polysaccharide)

| | | Location or Cell- | |
|---|---|---|---|
| No | Target receptor/protein | type in brain | Reference |
| 1 | Myelin Oligodendrocyte | Oligodendrocyte | WO2018123979A1 |
| | glycoprotein | | |
| 2 | Neuroglycan C or Chondroitin | Various brain | WO2020004490A1 |
| | sulfate proteoglycan 5 (CSPGS) | cells | |
| 3 | IGSF4B/SynCAM3/Cell adhesion | Various brain | WO2020004492A1 |
| | molecule 3 (CADM3) | cells | |
| 4 | CNPase (2′,3′-cyclic | Oligodendrocyte | Brain cell surface |
| | nucleotide 3″ | | marker (https:// |
| | phosphodiesterase) | | www.cellsignal.com/ |
| 5 | Myelin associated Glycoprotein | Oligodendrocyte | pathways/neuronal-and- |
| 6 | Myelin Basic Protein | Oligodendrocyte | glial-cell-markers) |
| 7 | EAATI (Solute Carrier Family 1 | Astrocyte | |
| | member3) | | |
| 8 | EAAT2 (Solute Carrier Family 1 | Astrocyte | |
| | member2) | | |
| 9 | MAP2 (Microtubule-associated | Neuron | |
| | protein2) | | |
| 10 | NEFL (Neurofilament light | Neuron | |
| | polypeptide) | | |
| 11 | NEFM (Neurofilament medium | Neuron | |
| | polypeptide) | | |
| 12 | NSE (Gamma-enolase) | Neuron | |
| 13 | CD68 (Macrosialin) | Microglia | |
| 14 | IBA1 or AIF1 (Allograft | Microglia | |
| | inflammatory factor1) | | |
| 15 | P2RY12 (Purinergic receptor | Microglia | |
| | P2Y12) | | |
| 16 | ILIRAPL1 (Interleukin 1 | Various brain | Protein that broadly |
| | receptor accessory protein | cells | expresses in brain |
| | like1) | | selected from Human |
| 17 | GRIN2B(Glutamate ionotropic | Various brain | protein Atlas (Uhlén M. |
| | receptor NMDA type subunit2B) | cells | et al., Tissue-based map |
| 18 | CACNG8(Calcium voltage-gated | Various brain | of the human proteome. |
| | channel auxiliary subunit | cells | Science (2015) |
| | gamma 8) | | |
| 19 | CD11b (Integrin subunit | Microglia | Brain cell surface |
| | alpha M) | | marker (https:// |
| | | | www.cellsignal.com/ |
| | | | pathways/neuronal-and- |
| | | | glial-cell-markers) |
| 20 | SLC6A2 (Sodium-dependent | Neuron | Protein that broadly |
| | noradrenaline transporter) | | expresses in brain |
| 21 | DPP6 (Dipeptidyl peptidase | Various brain | selected from Human |
| | like 6) | cells | protein Atlas (Uhlén M |
| 22 | SLC18A3 (Vesicular acetylcholine | Neuron | et al., Tissue-based map |
| | transporter) | | of the human proteome. |
| | | | Science (2015) |
| 23 | Sodium/potassium-transporting | Various brain | |
| | ATPase subunit alpha-2 | cells | |
| 24 | Broad substrate specificity | Microglia | |
| | ATP-binding cassette | | |
| | transporter ABCG2 | | |
| 25 | Solute carrier family 12 | Various brain | |
| | member 9 | cells | |
| 26 | Electrogenic sodium bicarbonate | Astrocyte | |
| | cotransporter 1 | | |
| 27 | Excitatory amino acid | Astrocyte | |
| | transporter 2 | | |
| 28 | Chondroitin sulfate | Oligodendrocyte | |
| | proteoglycan 4 | precursor cells | |
| 29 | Immunoglobulin superfamily | Various brain | |
| | DCC subclass member 4 | cells | |
| 30 | Vang-like protein 2 | Various brain | |
| | | cells | |
| 31 | Neural cell adhesion molecule | Various brain | |
| | 1 (N-CAM-1) | cells | |
| 32 | Low-density lipoprotein | Various brain | |
| | receptor-related protein | cells | |
| | 4 (LRP-4) | | |
| 33 | Phosphoprotein associated | Microglia | |
| | with glycosphingolipid- | | |
| | enriched microdomains 1 | | |
| | (Csk-binding protein) | | |
| 34 | Plasma membrane calcium- | Neuron | |
| | transporting ATPase 1 | | |
| 35 | Prominin-1 | Various brain | |
| | | cells | |
| 36 | Somatostatin receptor | Various brain | |
| | type 1 | cells | |
| 37 | Carnitine O-palmitoyl- | Various brain | |
| | transferase 1, | cells | |
| | brain isoform (CPT1-B) | | |
| 38 | Epidermal growth factor | Various brain | |
| | receptor | cells | |
| 39 | Protein MAL2 | Various brain | |
| | | cells | |
| 40 | Syntaxin-1A | Various brain | |
| | | cells | |
| 41 | Sodium/calcium exchanger 1 | Various brain | |
| | | cells | |
| 42 | Lysophosphatidylcholine | Various brain | |
| | acyltransferase 1 (IPC | cells | |
| | acyltransferase 1) | | |
| 43 | Calsyntenin-3 (Alcadein-beta) | Neuron | |
| 44 | Pituitary adenylate cyclase- | Various brain | |
| | activating polypeptide type | cells | |
| | I receptor (PACAP type I | | |
| | receptor) | | |
| 45 | Neutral cholesterol ester | Neuron | |
| | hydrolase 1 (NCEH) | | |
| 46 | CD166 antigen (Activated | Oligodendrocyte | |
| | leukocyte cell adhesion | | |
| | molecule) | | |
| 47 | Inactive tyrosine-protein | Various brain | |
| | kinase 7 | cells | |
| 48 | Claudin-11 | Myelin | Nat Commun. 2018 |
| 49 | Ectonucleotide phosphatase | Myelin | October 12; 9(1): 4230 |
| | (ENPP6) | | |
| 50 | Tetraspanin-2 (Tspan-2) | Myelin | |
| 51 | Myelin proteolipid protein | Myelin | |
| | (PLP) | | |
| 52 | Glycolipid transfer protein | Myelin | |
| | (GLTP) | | |
| 53 | Versican core protein | ECM | |
| | (Chondroitin sulfate | | |
| | proteoglycan 2) (CSPG2) | | |
| 54 | Tropoelastin (Elastin) | ECM | |
| 55 | Collagen alpha-2(IV) chain | ECM | |
| | (Canstatin) | | |
| 56 | Proteoglycan link protein 1 | ECM | |
| | (Hyaluronan and proteoglycan | | |
| | link protein 1) | | |
| 57 | Tenascin-R (TN-R) | ECM | |
| 58 | Proteoglycan link protein 2 | ECM | |
| | (Hyaluronan and proteoglycan | | |
| | link protein 2) | | |
| 59 | Collagen alpha-1(I) chain | ECM | |
| 60 | Neurofilament-3 (NEF3) | Cell | Cell. 2013 August |
| | | membrane/ECM | 29; 154(5): 971-982 |
| 61 | Immunoglobulin superfamily | Cell | |
| | member 8 (lgSF8) | membrane/ECM | |
| 62 | Laminin subunit gamma-1 | ECM | |
| | (LAMCI) | | |
| 63 | Collagen alpha-1(VI) chain | ECM | |
| | (Col6al) | | |
| 64 | Collagen alpha-3(VI) chain | ECM | |
| | (Col6a3) | | |

### Example 2. Preparation of Molecule Having a Brain Transfer Moiety and a Brain Retention Moiety

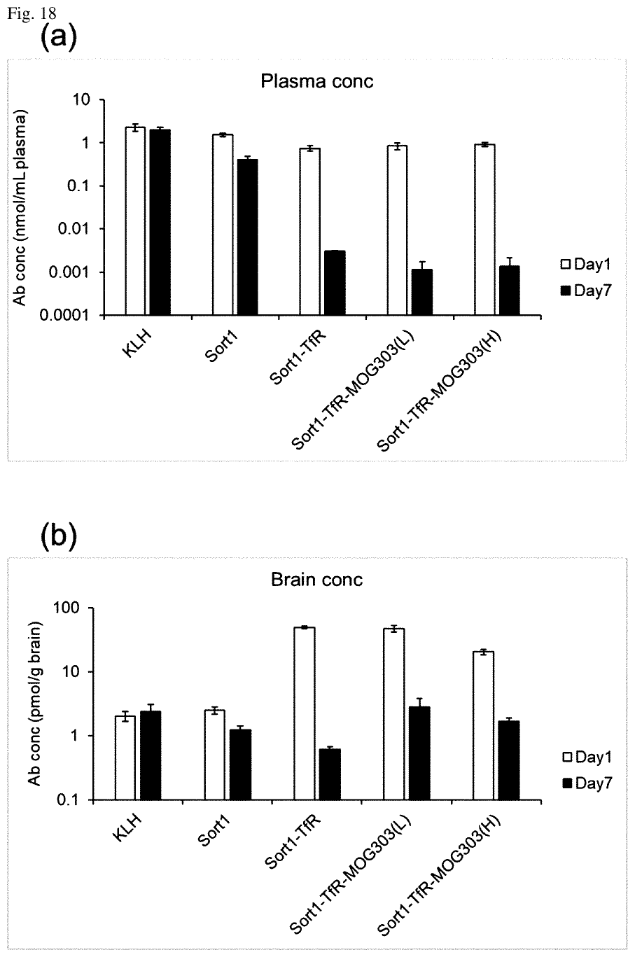

An antibody which comprises a transferrin receptor (TfR)-binding domain (brain transfer moiety) and two myelin oligodendrocyte glycoprotein (MOG)-binding domains (brain retention moiety), named as MOG303/TfR, was generated as described below. The molecular format of MOG303/TfR is showed in FIG. 11(A).

MOG303, a bivalent antibody which comprises two myelin oligodendrocyte glycoprotein (MOG)-binding domains; KLH, a bivalent anti-KLH (keyhole limpet hemocyanin) antibody; and KLH/TfR, an antibody which comprises a KLH-binding domain and a TfR-binding domain; were also prepared by a method known in the art. Both MOG303 and KLH comprise heavy chain constant region of modified hIgG1 (SEQ ID NO: 2).

Expression vectors encoding heavy chains and light chains of MOG303/TfR, MOG303, KLH-TfR and KLH as shown in Table 3 were constructed by a method known in the art.

TABLE 3
Expression vectors, heavy chains and light chains
of MOG303//TfR. MOG303, KLH-TfR and KLH

| Antibody | Heavy chain 1 | Heavy chain 2 | Light chain 1 & 2 |
|---|---|---|---|
| MOG303//TfR | MOG303VH- | MOG303VH- | MOG303VL-SK1 |
| | SG181v11k.newmBBB | SG181v11h | (SEQ ID NO: 8) |
| | (SEQ ID NO: 9) | (SEQ ID NO: 10) | |
| MOG303 | MOG303VH-SG181 | MOG303VH-SG181 | MOG303VL-SK1 |
| | (SEQ ID NO: 7) | (SEQ ID NO: 7) | (SEQ ID NO: 8) |
| KLH-TfR | IC17HdK- | IC17HdK-SG181v11h | IC17L-SK1 |
| | SG181v11k.newmBBB | (SEQ ID NO: 12) | (SEQ ID NO:13) |
| | (SEQ ID NO: 11) | | |
| KLH | IC17HdK-SG181 | IC17HdK-SG181 | IC17L-SK1 |
| | (SEQ ID NO: 14) | (SEQ ID NO: 14) | (SEQ ID NO: 13) |

An expression vector encoding MOG303VH-SG181v11k.newmBBB (heavy chain 1: SEQ ID NO: 9) containing VH region of anti-MOG antibody (SEQ ID NO: 1), a modified human IgG1 constant region (CH1-hinge-CH2-CH3, decreased binding to the activating F gamma receptors (human Fc gamma RIa, human Fc gamma RIIa(R), human Fc gamma RIIa(H), human Fc gamma RIIIa(V), and human Fc gamma RIIIa(F)), and comprises mutations for heterodimeric Fc production) and single chain Fab domain of anti-mouse Transferrin receptor antibody followed by Gly-Ser linker (Constant region to scFab: SEQ ID NO: 5) was prepared by a method known in the art.

An expression vector encoding MOG303VH-SG181v11h (heavy chain 2: SEQ ID NO: 10) containing VH region of anti-MOG antibody (SEQ ID NO: 1), a modified human IgG₁ constant region (CH1-hinge-CH2-CH3, decreased binding to the activating F gamma receptors (human Fc gamma RIa, human Fc gamma RIIa(R), human Fc gamma RIIa(H), human Fc gamma RIIIa(V), and human Fc gamma RIIIa(F) and comprises mutations for heterodimeric Fc production; SEQ ID NO: 6) was prepared by a method known in the art.

An expression vector encoding MOG303VL-SK1 (light chain: SEQ ID NO: 8) containing VL region of anti-MOG antibody (SEQ ID NO: 3) fused with human kappa constant region (SEQ ID NO: 4) was prepared by a method known in the art.

Expression of MOG303/TfR antibody (heavy chain 1: SEQ ID NO: 9; heavy chain 2: SEQ ID NO: 10; light chain 1 and 2 sequence: SEQ ID NO: 8) which has a molecular format of as showed in FIG. 11(A) was done by using HEK293 cell expression system. Heavy chain 1, Heavy chain 2 and Light chain 1 and 2 were transfected to Expi293 cell according to manufacturer's protocol. After culturing of several days the cultured medium was collected and subjected to the affinity column. The antibody proteins were purified by affinity column (Protein A) and were subjected to size exclusion chromatography to obtain MOG303/TfR. The expression and purification methods were conducted by a method known in the art (Nat Protoc. 2018 January; 13(1):99-117).

### Example 3. Pharmacokinetic Profiles of an Antibody in Brain and in Plasma in Mice

Pharmacokinetics of anti-KLH antibodies and anti-MOG antibodies with or without an anti-TfR Fab domain in brain and in plasma were evaluated in C57BL/6J mice (male, 6-8 weeks).

IgG antibodies, KLH-IgG (KLH) and MOG303-IgG (MOG303), and IgG antibodies with an anti-TfR domain, KLH/TfR and MOG303/TfR were administered intravenously to mice at a dose of 2 mg/kg. Blood was collected from 1 day to 28 days after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. 50 mg of the pieces of minced brain was dissolved by PBS containing NP-40 alternative detergent (Millipore) and Complete Mini Protease Inhibitor Cocktail (Roche) with 5 mm stainless beads, and was homogenized with a homogenizer (Qiagen). After rotation at 4 degrees C. for 60 min, samples were centrifuged (15,000 rpm, 4 degrees C., 20 min) and the supernatant was collected.

Concentrations of the antibodies in plasma and brain were determined by the electro chemiluminescence-immunoassay (ECL). As a capture antibody, an anti-human IgG Fab′2 antibody (LifeSpan BioSciences) was applied in the plate. After PBS with Tween20 (PBST, Sigma-Aldrich) containing 1% bovine serum albumin (Sigma-Aldrich) was added as blocking solution, diluted plasma and brain homogenate samples were applied. As a detection antibody, a biotin-labeled anti-human IgG antibody (Bethyl Laboratories) was added. Finally, a streptavidin labeled with SULFO-tag (Meso Scale Discovery) was added, and signals were detected by ECL (Meso Scale Discovery). The concentration-time profiles of the antibodies in plasma and brain were analyzed by non-compartment model using Phoenix WinNonlin (ver. 8.3) to reveal the area under the curve (AUC), clearance, the volume of distribution (Vd), and half-life. The brain/plasma ratio was calculated by dividing the concentrations in brain by those in plasma. The percent of injected dose per brain tissue weight was calculated by dividing the concentrations in brain by dose amount.

The pharmacokinetic profiles of KLH, MOG303, KLH/TfR and MOG303/TfR in brain were shown in FIG. 3. At day 1 after dosing, control antibody KLH showed around 0.046 microgram/g antibody concentration in brain, 0.003 brain-to-plasma concentration ratio, and 0.10% ID/g brain (i.e. percent of injected dose per brain tissue weight). The concentration at day 1 was the maximum concentrations (Cmax) during the period of study for KLH. MOG303 showed similar antibody concentration and brain distribution profile with KLH at day 1. On the other hand, KLH/TfR showed 1.4 microgram/g antibody concentration in brain, 0.37 brain-to-plasma concentration ratio and 3.1% ID/g brain, which were higher than that of KLH and MOG303. The concentration of KLH/TfR at day 1 was the Cmax during the period of study. Surprisingly, MOG303/TfR showed around 2-fold higher concentration in brain, brain-to-plasma concentration ratio and % ID/g brain (2.8 microgram/g brain, 0.55 brain-to-plasma concentration ratio, 6.1% ID/g brain) than that of KLH/TfR at day 1. These results indicate that combination of the transfer moiety (anti-TfR) and the retention moiety (anti-MOG) contributes to significant increased antibody concentration and distribution in brain, within short duration after antibody injection (1 day).

At day 7 to day 28, the brain concentrations and the percent of dose of KLH decreased gradually (FIG. 3(a)), and brain-to-plasma ratio was almost constant (FIG. 3(b)). The brain concentrations, brain-to-plasma ratio, and the percent of dose of KLH/TfR disappeared very rapidly. The concentrations after day 14 were below that of KLH and the concentration at day 28 was below the limit of quantification (<0.0013 microgram/g). Half-life of KLH/TfR in brain was 8.77 day. These results suggest that the antibody transferred into brain was eliminated by CSF/ISF bulk flow and reverse transcytosis to plasma. On the other hand, MOG303 accumulated in brain in the time-dependent manner due to binding to MOG protein in brain after transfer into brain. The concentration of MOG303 reached the Cmax at day 28 at last (0.32 microgram/g and 0.70% ID/g). Interestingly, MOG303/TfR showed no significant reduction of antibody concentration in brain over time and remained high concentration and distribution in brain starting from day 1 until at least day 28 (2.2-3.1 microgram/g and 4.7-7.0% ID/g). The Cmax was 3.1 microgram/g and 7.0% ID/g during the period of study.

The observed high starting antibody concentration at day 1 and high retention of MOG303/TfR in brain for at least 28 days is not foreseeable given that antibodies having TfR-binding domain was reported to be quickly eliminated from the brain, which is also observed in our tested control antibody having anti-TfR domain i.e. KLH/TfR (see FIG. 3(a), (b), (c)). These data suggest that the combination of the transfer moiety (anti-TfR) and retention moiety (anti-MOG) provides high antibody concentration and longer lasting in brain.

Unexpectedly, MOG303/TfR showed synergistic effect in terms of cumulative brain AUC (area under the curve) which represents the total antibody exposure in brain across a time interval, wherein the cumulative brain AUC of MOG303/TfR is approximately 108-fold, 15.2-fold, and 24.7-fold higher than that of KLH, MOG303, and KLH/TfR, respectively (FIG. 3(d)). Notably, the cumulative brain AUC of MOG303/TfR (73.0 microgram/g*day) is greater than the sum of cumulative brain AUC of each of MOG303 (4.80 microgram/g*day) and KLH/TfR (2.95 microgram/g*day). The results suggest that the combination of the transfer moiety (anti-TfR) and retention moiety (anti-MOG) provides synergistic effect of longer lasting and high antibody concentration in brain.

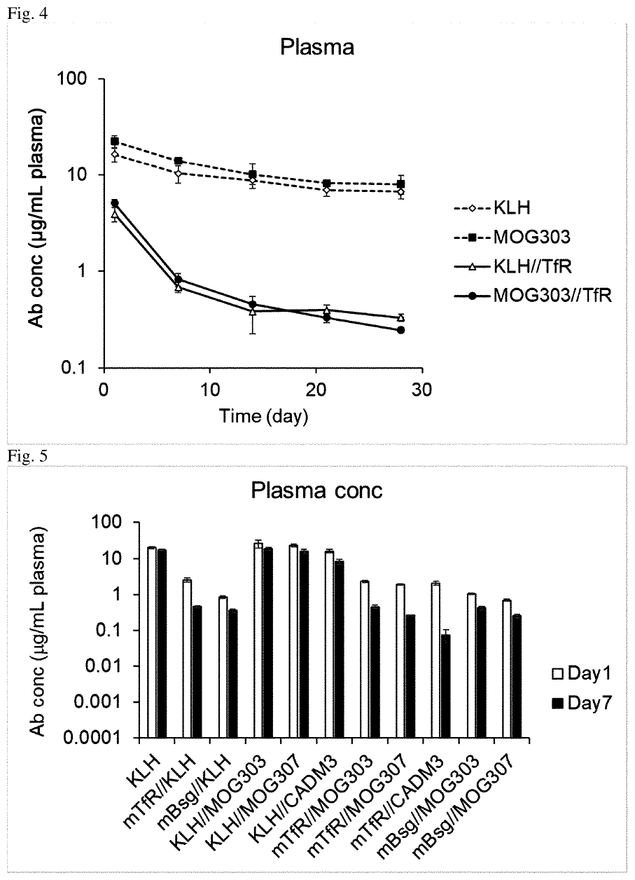

The pharmacokinetic (PK) profiles of KLH, MOG303, KLH/TfR and MOG303/TfR in plasma were shown in FIG. 4. Similar PK profiles in plasma were found for both KLH and MOG303. On the other hand, KLH/TfR and MOG303/TfR showed lower concentrations starting from day 1, and were eliminated much faster than KLH and MOG303. As shown in Table 4, the clearance of KLH/TfR and MOG303/TfR was more than 10-fold higher than that of KLH and MOG303. These results suggest that antibodies with anti-TfR domain were eliminated from plasma rapidly due to the systemic expression of TfR.

TABLE 4
Pharmacokinetic parameters of the antibodies in plasma
AUC (0-inf), clearance, Vd, and half-life of KLH, MOG303, KLH//TfR
and MOG303//TfR in plasma were calculated by non-compartment model
analysis. AUC and Vd mean the area under the curve and the volume of
distribution, respectively.

| | AUC (0-inf) | Clearance | Vd | Half-life |
|---|---|---|---|---|
| Antibody | μg*day/mL | mL/day/kg | mL/kg | day |
| KLH | 586 | 3.41 | 159 | 33.1 |
| MOG303 | 612 | 3.27 | 112 | 23.8 |
| KLH//TfR | 39.1 | 51.1 | 1609 | 30.6 |
| MOG303//TfR | 34.6 | 57.8 | 786 | 15.8 |

### Example 4. Preparation of Additional Bispecific Antibodies Having a Brain Transfer Moiety and a Brain Retention Moiety

### Example 4A: Bispecific Antibodies Each Comprising a Brain Transfer Moiety (i.e. Anti-Transferrin Receptor Antibody or Anti-Basigin Antibody) and a Brain Retention Moiety (i.e. Anti-MOG Antibody, Anti-CSPG5 Antibody or Anti-CADM3 Antibody)

Bispecific antibodies each comprising a brain transfer moiety (i.e. anti-transferrin receptor antibody or anti-basigin antibody) and a brain retention moiety (i.e. anti-MOG antibody, anti-CSPG5 antibody or anti-CADM3 antibody) were generated as described below.

Bivalent antibodies comprising two brain transfer moieties were prepared. Anti-transferrin receptor antibody, mTfR (H chain variable region: SEQ ID NO: 29, L chain variable region: SEQ ID NO: 30) and anti-Basigin antibody, mBsg (H chain variable region: SEQ ID NO: 31, L chain variable region: SEQ ID NO: 32) were prepared. Each of the antibodies comprises heavy chain constant region of modified hIgG1 (SEQ ID NO: 33) and light chain constant region of human kappa region (SEQ ID NO: 4).

Bivalent antibodies comprising two brain retention moieties were prepared. Anti-MOG antibodies, MOG303 (H chain variable region: SEQ ID NO: 15, L chain variable region: SEQ ID NO: 17) and MOG307 (H chain variable region: SEQ ID NO: 34, L chain variable region: SEQ ID NO: 35); anti-Chondroitin sulfate proteoglycan 5 (CSPG5) antibody, CSPG5.2 (H chain variable region: SEQ ID NO: 36, L chain variable region: SEQ ID NO: 37); and anti-CADM3 antibody, CADM3 (H chain variable region: SEQ ID NO: 38, L chain variable region: SEQ ID NO: 39) were prepared. Each of the antibodies comprise heavy chain constant region of modified hIgG1 (SEQ ID NO: 40) and light chain constant region of human kappa region (SEQ ID NO: 4). As control antibodies, bivalent anti-KLH antibodies (KLHn, KLHp) were generated and comprise either modified hIgG1 and human kappa region.

Expression vectors encoding heavy chains and light chains of bivalent antibodies of transfer moiety or bivalent antibody of retention moiety as shown in Table 5 were constructed by a method known in the art. All antibodies were transiently expressed in mammalian cells by the method known to those skilled in the art using the genes constructed and were purified by the method known to those skilled in the art.

TABLE 5

| Antibody | Heavy chain | Light chain |
|---|---|---|
| mTfR | 8D3VH-SG181.S3p | 8D3VL-SK1 |
| | (SEQ ID NO: 41) | (SEQ ID NO: 49) |
| mBsg | BsgAVH-SG181.S3p | BsgAVL-SK1 |
| | (SEQ ID NO: 42) | (SEQ ID NO: 50) |
| MOG303 | MOG303VH-SG181.S3n | MOG303VL-SK1 |
| | (SEQ ID NO: 43) | (SEQ ID NO: 51) |
| MOG307 | MOG307VH-SG181.S3n | MOG307VL-SK1 |
| | (SEQ ID NO: 44) | (SEQ ID NO: 52) |
| CSPG5.2 | CSPG5202VH-SG181.S3n | CSPG5202VL-SK1 |
| | (SEQ ID NO: 45) | (SEQ ID NO: 53) |
| CADM3 | CADM3501VH-SG181.S3n | CADM3501VL-SK1 |
| | (SEQ ID NO: 46) | (SEQ ID NO: 54) |
| KLHn | IC17HdK-SG181.S3n | IC17L-SK1 |
| | (SEQ ID NO: 47) | (SEQ ID NO: 55) |
| KLHp | IC17HdK-SG181.S3p | C17L-SK1 |
| | (SEQ ID NO: 48) | (SEQ ID NO: 55) |

The bivalent antibodies were then subjected to Fab-arm exchange as described in WO2016/159213 to generate bispecific antibodies as listed below (see Table 6 for the sequences of the heavy and light chains):
a) mTfR/KLH (bispecific antibody of anti-mouse Transferrin receptor antibody and anti-KLH antibody),
b) mBsg/KLH (bispecific antibody of anti-mouse basigin antibody and anti-KLH antibody),
c) KLH/MOG303 (bispecific antibody of anti-mouse MOG antibody and anti-KLH antibody),
d) KLH/MOG307 (bispecific antibody of anti-mouse MOG antibody and anti-KLH antibody),
e) KLH/CSPG5.2 (bispecific antibody of anti-mouse CSPG5 antibody and anti-KLH antibody),
f) KLH/CADM3 (bispecific antibody of anti-mouse CADM3 antibody and anti-KLH antibody),
g) mTfR/MOG303 (bispecific antibody of anti-mouse Transferrin receptor antibody and anti-mouse MOG antibody),
h) mTfR/MOG307 (bispecific antibody of anti-mouse Transferrin receptor antibody and anti-mouse MOG antibody),
i) mTfR/CSPG5.2 (bispecific antibody of anti-mouse Transferrin receptor antibody and anti-CSPG5 antibody),
j) mTfR/CADM3 (bispecific antibody of anti-mouse Transferrin receptor antibody and anti-mouse CADM3 antibody),
k) mBsg/MOG303 (bispecific antibody of anti-mouse basigin antibody and anti-mouse MOG antibody),
l) mBsg/MOG307 (bispecific antibody of anti-mouse basigin antibody and anti-mouse MOG antibody),
m) mBsg/CSPG5.2 (bispecific antibody of anti-mouse basigin antibody and anti-mouse CSPG5 antibody).

The exemplary bispecific antibodies listed above are monovalent for the brain transfer moiety and are monovalent for the brain retention moiety. FIG. 11B shows a schematic drawing of the molecular format of the bispecific antibodies (f) to (m) as listed above.

TABLE 6

| | Brain | Brain | Brain | Brain |
|---|---|---|---|---|
| | transfer | transfer | retention | retention |
| Bispecific | moiety | moiety | moiety | moiety |
| antibody | (heavy chain) | (light chain) | (heavy chain) | (light chain) |
| mTfR//KLH | 8D3VH- | 8D3VL-SK1 | IC17HdK- | IC17L-SK1 |
| | SG181.S3p | (SEQ ID NO: | SG181.S3n | (SEQ ID NO: |
| | (SEQ ID NO: | 49) | (SEQ ID NO: | 55) |
| | 41) | | 47) | |
| mBsg//KLH | BsgAVH- | BsgAVL-SK1 | IC17HdK- | ICI7L-SK1 |
| | SG181.S3p | (SEQ ID NO: | SG181.S3n | (SEQ ID NO: |
| | (SEQ ID NO: | 50) | (SEQ ID NO: | 55) |
| | 42) | | 47) | |
| KLH//MOG303 | IC17HdK- | IC17L-SK1 | MOG303VH- | MOG303VL- |
| | SG181.S3p | SEQ ID NO: | SG181.S3n | SK1 |
| | (SEQ ID NO: | 55) | (SEQ ID NO: | (SEQ ID NO: |
| | 48) | | 43) | 51) |
| KLH//MOG307 | IC17HdK- | IC17L-SK1 | MOG307VH- | MOG307VL- |
| | SG181.S3p | SEQ ID NO: | SG181.S3n | SK1 |
| | (SEQ ID NO: | 55) | (SEQ ID NO: | (SEQ ID NO: |
| | 48) | | 44) | 52) |
| KLH//CSPG5.2 | IC17HdK- | IC17L-SK1 | CSPG5202VH- | CSPG5202VL- |
| | SG181.S3p | (SEQ ID NO: | SG181.S3n | SK1 |
| | (SEQ ID NO: | 55) | (SEQ ID NO: | (SEQ ID NO: |
| | 48) | | 45) | 53) |
| KLH//CADM3 | IC17HdK- | IC17L-SK1 | CADM3501VH- | CADM3501VL- |
| | SG181.S3p | (SEQ ID NO: | SG181.S3n | SK1 |
| | (SEQ ID NO: | 55) | (SEQ ID NO: | (SEQ ID NO: |
| | 48) | | 46) | 54) |
| mTfR//MOG303 | 8D3VH- | 8D3VL-SK1 | MOG303VH- | MOG303VL- |
| | SG181.S3p | (SEQ ID NO: | SG181.S3n | SK1 |
| | (SEQ ID NO: | 49) | (SEQ ID NO: | (SEQ ID NO: |
| | 41) | | 43) | 51) |
| mTfR//MOG307 | 8D3VH- | 8D3VL-SK1 | MOG307VH- | MOG307VL- |
| | SG181.S3p | (SEQ ID NO: | SG181.S3n | SK1 |
| | (SEQ ID NO: | 49) | (SEQ ID NO: | (SEQ ID NO: |
| | 41) | | 44) | 52) |
| mTfR//CSPG5.2 | 8D3VH- | 8D3VL-SK1 | CSPG5202VH- | CSPG5202VL- |
| | SG181.S3p | (SEQ ID NO: | SG181.S3n | SK1 |
| | (SEQ ID NO: | 49) | (SEQ | (SEQ ID NO: |
| | 41) | | ID NO: | 53) |
| | | | 45) | |
| mTfR//CADM3 | 8D3VH- | 8D3VL-SK1 | CADM3501VH- | CADM3501VL- |
| | SG181.S3p | (SEQ ID NO: | SG181.S3n | SK1 |
| | (SEQ ID NO: | 49) | (SEQ ID NO: | (SEQ ID NO: |
| | 41) | | 46) | 54) |
| mBsg//MOG303 | BsgAVH- | BsgA VL-SK1 | MOG303VH- | MOG303VL- |
| | SG181.S3p | SEQ ID NO: | SG181.S3n | SK1 |
| | (SEQ ID NO: | 50) | (SEQ ID NO: | (SEQ ID NO: |
| | 42) | | 43) | 51) |
| mBsg//MOG307 | BsgAVH- | BsgAVL-SK1 | MOG307VH- | MOG307VL- |
| | SG181.S3p | (SEQ ID NO: | SG181.S3n | SK1 |
| | (SEQ ID NO: | 50) | (SEQ ID NO: | (SEQ ID NO: |
| | 42) | | 44) | 52) |
| mBsg//CSPG5.2 | BsgAVH- | BsgAVL-SK1 | CSPG5202VH- | CSPG5202VL- |
| | SG181.S3p | (SEQ ID NO: | SG181.S3n | SK1 |
| | (SEQ ID NO: | 50) | (SEQ ID NO: | (SEQ ID NO: |
| | 42) | | 45) | 53) |
| mBsg//CADM3 | BsgAVH- | BsgAVL-SK1 | CADM3501VH- | CADM3501VL- |
| | SG181.S3p | (SEQ ID NO: | SG181.S3n | SK1 |
| | (SEQ ID NO: | 50) | (SEQ ID NO: | (SEQ ID NO: |
| | 42) | | 46) | 54) |

As described above, bispecific antibody was generated by Fab-arm exchange technology. Alternatively, it can also be generated by transfecting two different heavy and two different light chains plasmids into mammalian cells.

To efficiently obtain a bispecific antibody of interest, there are known amino acid substitutions and combinations in the CH1-CL domain interface that promote desired H chain-L chain association (such as e.g. described in WO2019065795) that can be used in an embodiment.

### Example 4B: Bispecific Molecule Having Anti-Transferrin Receptor Antibody and Anti-CSPG5 Antibody

An antibody which comprises a transferrin receptor (TfR)-binding domain (brain transfer moiety) and two Chondroitin sulfate proteoglycan 5 (CSPG5)-binding domains (brain retention moiety), named as CSPG5120-BS, was generated as described below. The molecular format of CSPG5120-BS is showed in FIG. 11(A).

CSPG5, a bivalent antibody which comprises two Chondroitin sulfate proteoglycan 5 (CSPG5)-binding domains, was also prepared by a method known in the art. CSPG5 comprises heavy chain constant region of modified hIgG1 (SEQ ID NO: 40).

Expression vectors encoding heavy chains and light chains of CSPG5120-BS, CSPG5120 and KLH as shown in Table 7 were constructed by a method known in the art.

TABLE 7

| | | | Light chain |
|---|---|---|---|
| Antibody | Heavy chain 1 | Heavy chain 2 | 1 & 2 |
| CSPG5120-BS | CSPGSVH- | CSPGSVH - | CSPG5VL-SK1 |
| | SG181v11k.newm.BBB | SG181v11h | (SEQ ID NO: 60) |
| | (SEQ ID NO: 58) | (SEQ ID NO: 59) | |
| CSPG5120 | CSPG5VH-SG181.S3n | CSPG5VH-SG181.S3n | CSPGSVL-SK1 |
| | (SEQ ID NO: 61) | (SEQ ID NO: 61) | (SEQ ID NO: 60) |
| KLH | IC17HdK-SG181 | IC17HdK-SG181 | IC17L-SK1 |
| | (SEQ ID NO: 14) | (SEQ ID NO: 14) | (SEQ ID NO: 13) |

An expression vector encoding CSPG5VH-SG181v11k.newmBBB (heavy chain 1: SEQ ID NO: 58) containing VH region of anti-CSPG5 antibody (SEQ ID NO: 56), a modified human IgG1 constant region (CH1-hinge-CH2-CH3, decreased binding to the activating F gamma receptors (human Fc gamma RIa, human Fc gamma RIIa(R), human Fc gamma RIIa(H), human Fc gamma RIIIa(V), and human Fc gamma RIIIa(F)) and comprises mutations for heterodimeric Fc production) and single chain Fab domain of anti-mouse Transferrin receptor antibody followed by Gly-Ser linker (Constant region to scFab: SEQ ID NO: 5) was prepared by a method known in the art.

An expression vector encoding CSPG5VH-SG181v11h (heavy chain 2: SEQ ID NO: 10) containing VH region of anti-CSPG5 antibody (SEQ ID NO: 56), a modified human IgG1 constant region (CH1-hinge-CH2-CH3, decreased binding to the activating F gamma receptors (human Fc gamma RIa, human Fc gamma RIIa(R), human Fc gamma RIIa(H), human Fc gamma RIIIa(V), and human Fc gamma RIIIa(F) and comprises mutations for heterodimeric Fc production; SEQ ID NO: 6) was prepared by a method known in the art.

An expression vector encoding CSPG5VL-SK1 (light chain: SEQ ID NO: 60) containing VL region of anti-CSPG5 antibody (SEQ ID NO: 57) fused with human kappa constant region (SEQ ID NO: 4) was prepared by a method known in the art.

Expression of CSPG5120-BS antibody (heavy chain 1: SEQ ID NO: 58; heavy chain 2: SEQ ID NO: 59; light chain sequence: SEQ ID NO: 60) having a molecular format as shown in FIG. 11(A) was done by using HEK293 cell expression system. Heavy chain 1, Heavy chain 2 and Light chain were transfected to Expi293 cell according to manufacturer's protocol. After culturing of several days the cultured medium was collected and subjected to the affinity column. The antibody proteins were purified by affinity column (Protein A) and were subjected to size exclusion chromatography to obtain CSPG5120-BS. The expression and purification methods were conducted by a method known in the art (Nat Protoc. 2018 January; 13(1):99-117).

### Example 5. Pharmacokinetic Profiles of Antibodies in Brain and in Plasma in Mice

Pharmacokinetics of the antibodies prepared in Example 4 were evaluated in C57BL/6J mice (male, 6-8 weeks) to reveal elimination from plasma, and transferability into and retention of the antibodies in brain.

The negative control antibody, KLH, the antibodies with brain transfer moiety, mTfR/KLH, mBsg/KLH, the bispecific antibodies with brain retention moiety, KLH/MOG303, KLH/MOG307, KLH/CADM3, and the antibodies with both brain transfer moiety and brain retention moiety, mTfR/MOG303, mTfR/MOG307, mTfR/CADM3, mBsg/MOG303, mBsg/MOG307, were administered intravenously in mice at a dose of 2 mg/kg. Blood was collected on 1 day and 7 day after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. A half of the brain was dissolved by Cell extraction buffer (Invitrogen) containing Complete Mini Protease Inhibitor Cocktail (Roche) in M tube (Miltenyi), and was homogenized with gentle MACS (Miltenyi). Homogenate was centrifuged (15,000 rpm, 4 degrees C., 20 min) and the supernatant was collected.

Concentrations of the antibodies in brain and in plasma were determined by the electro chemiluminescence-immunoassay (ECL). As a capture antibody, an anti-human IgG Fab′2 antibody (LifeSpan BioSciences) was applied in the plate. After PBS with Tween20 (PBST, Sigma-Aldrich) containing 1% bovine serum albumin (Sigma-Aldrich) was added as blocking solution, diluted plasma or brain homogenate samples were applied. As a detection antibody, a biotin-labeled anti-human IgG antibody (Bethyl Laboratories) was added. Finally, a streptavidin labeled with SULFO-tag (Meso Scale Discovery) was added, and signals were detected by ECL (Meso Scale Discovery).

The brain/plasma ratio was calculated by dividing the concentrations in brain by those in plasma. The percent of injected dose per brain tissue weight (% ID/g) was calculated by dividing the concentrations in brain by dose amount.

The concentrations of the antibodies in plasma at day 1 and day 7 were shown in FIG. 5. mTfR/KLH and mBsg/KLH showed lower antibody concentration in plasma at day 1 than that of KLH, and the concentration in plasma continued to decrease at day 7. On the other hand, KLH/MOG303, KLH/MOG307 and KLH/CADM3 showed comparable concentrations with KLH at day 1 and day 7. mTfR/MOG303, mTfR/MOG307, mTfR/CADM3, mBsg/MOG303 and mBsg/MOG307 showed similar concentrations with mTfR/KLH and mBsg/KLH.

These results suggest that the antibodies with brain transfer moiety (in the examples: TfR and/or Bsg) were eliminated from the plasma rapidly due to the systemic expression of the brain transfer moiety (in the examples: TfR and/or Bsg), and the brain retention moiety (in the examples: MOG or CADM3) had minor contribution on the clearance of the antibodies from the plasma.

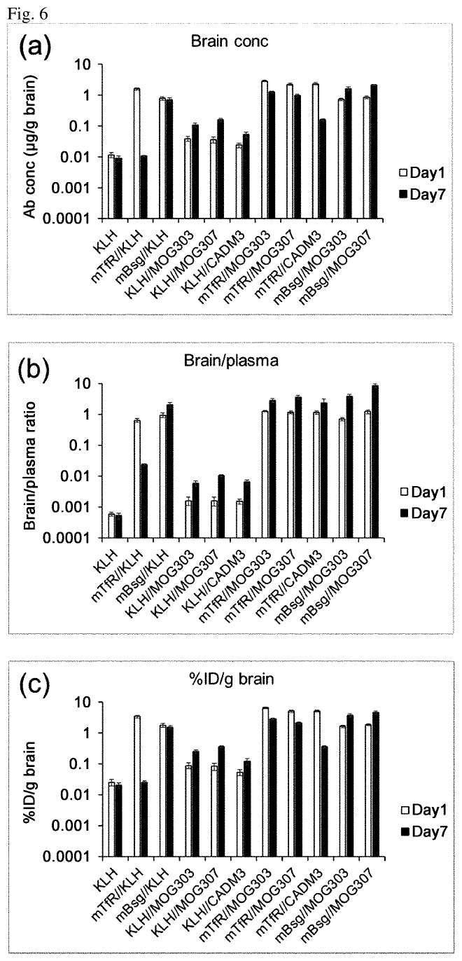

The concentrations of the antibodies in brain of mice at day 1 and day 7 were shown in FIG. 6(a), (b), (c). At day 1 and day 7, KLH showed around 0.01 microgram/g brain, 0.0005 brain-to-plasma ratio, 0.02% ID/g brain. mTfR/KLH showed high brain concentration at day 1 (3.4% ID/g brain), but the concentration decrease dramatically from day 1 to 7 (0.025% ID/g), suggesting that the antibody transferred into brain was eliminated rapidly by CSF/ISF bulk flow and reverse transcytosis to plasma. On the other hand, mBsg/KLH showed high concentrations both at day 1 and day 7, which indicates retention in brain. KLH/MOG303, KLH/MOG307 and KLH/CADM3 showed slightly higher concentration than KLH at day 1, and increased in brain until day 7 due to binding to MOG or CADM3 protein, respectively, and accumulating in brain after transfer into brain.

In addition, surprisingly, the combination of the brain transfer moiety and the brain retention moiety showed equivalent or higher concentrations at day 1 compared with the antibodies with the brain transfer moiety alone, and much higher concentrations at day 7 than the antibodies with the brain transfer moiety alone or the brain retention moiety alone. In the examples, this was shown for mTfR/MOG303 (i.e. a bispecific antibody comprising a brain transfer moiety binding to TfR and a brain retention moiety binding to MOG), mTfR/MOG307 (i.e. a bispecific antibody comprising a brain transfer moiety binding to TfR and a brain retention moiety binding to MOG), mTfR/CADM3 (i.e. a bispecific antibody comprising a brain transfer moiety binding to TfR and a brain retention moiety binding to CADM3), mBsg/MOG303 (i.e. a bispecific antibody comprising a brain transfer moiety binding to Basigin and a brain retention moiety binding to MOG) and mBsg/MOG307 (i.e. a bispecific antibody comprising a brain transfer moiety binding to Basigin and a brain retention moiety binding to MOG).

These data suggest that the combination of the brain transfer moiety and the brain retention moiety has a synergistic effect on the potency of retention of an antibody in brain.

### Example 6. Pharmacokinetic Profiles of Bispecific Antibodies mBsg/MOG303, mBsg/CSPG5.2 and mBsg/CADM3 in Brain and in Plasma in Mice

In another experiment, pharmacokinetics of the antibodies having anti-basigin antibody as brain retention moiety as prepared in Example 4 were evaluated in C57BL/6J mice (male, 6-8 weeks) to reveal elimination from plasma, and transferability into and retention of the antibodies in brain.

The negative control antibody, KLH, and the antibodies with brain transfer moiety, mBsg/KLH, and the bispecific antibodies with both brain transfer moiety and brain retention moiety, mBsg/MOG303, mBsg/CSPG5.2, mBsg/CADM3, were administered intravenously in C57BL/6J mice (male, 6-8 weeks) at a dose of 2 mg/kg. Blood and perfused brain were collected on 1 day and 7 day after dosing, and were treated as shown in the Example 5. In addition, the concentrations of the antibodies in plasma and brain were determined by the ECL as shown in the Example 5.

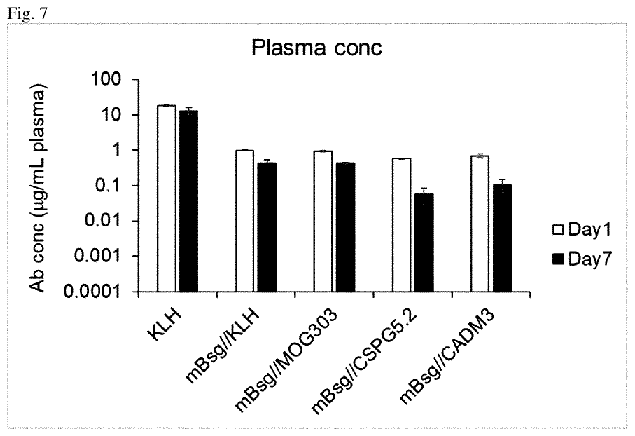

The concentrations in plasma at day 1 and day 7 were shown in FIG. 7. mBsg/KLH, mBsg/MOG303, mBsg/CSPG5.2 and mBsg/CADM3 showed lower concentrations at day 1 than KLH, and decreased until day 7. These results indicate that the antibodies with brain transfer moiety were disappeared rapidly due to the systemic expression of Bsg, and the retention moiety had minor contribution on the clearance of the antibodies.

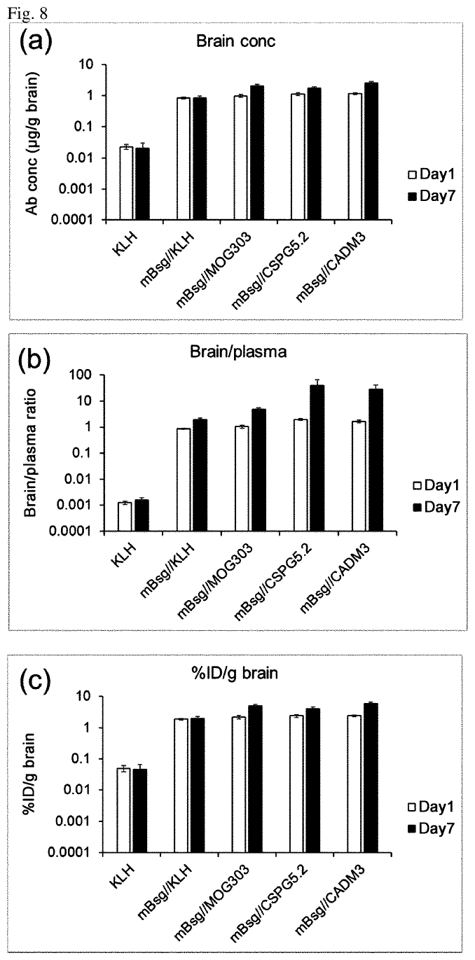

The concentrations in brain and brain pharmacodynamic data at day 1 and day 7 were shown in FIG. 8(a), (b), (c). mBsg/KLH showed higher concentrations at day 1 and day 7 than KLH, however both concentrations at day 1 and day 7 were comparable in mBsg/KLH. On the other hand, mBsg/MOG303, mBsg/CSPG5.2 and mBsg/CADM3 showed similar concentrations at day 1 as mBsg/KLH, and their concentrations increased from day 1 to day 7. At day 7, the concentrations for mBsg/MOG303, mBsg/CSPG5.2 and mBsg/CADM3 were higher than for mBsg/KLH.

These data suggest that the combination of a brain transfer moiety binding to Basigin (mBsg) and a brain retention moiety binding to MOG, CSPG5 or CADM3 has superior potency in the transfer and retention into the brain.

### Example 7. Pharmacokinetic Profiles of Bispecific Antibody CSPG5120-BS in Brain and in Plasma in Mice

The negative control antibody, KLH, and the anti-CSPG5 antibody, CSPG5120, and the CSPG5120 with the anti-TfR Fab domain (CSPG5120-BS) were administered intravenously in C57BL/6J mice (male, 6-8 weeks) at a dose of 2 mg/kg. Blood and perfused brain were collected on 1 day and 7 day after dosing, and were treated as shown in the Example 5. In addition, the concentrations of the antibodies in plasma and brain were determined by the ECL as shown in the Example 5.

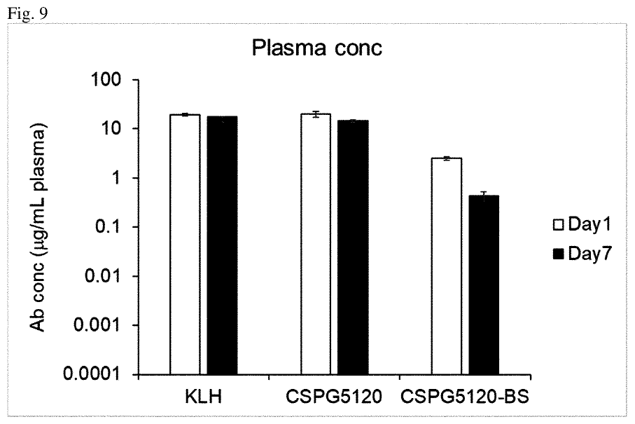

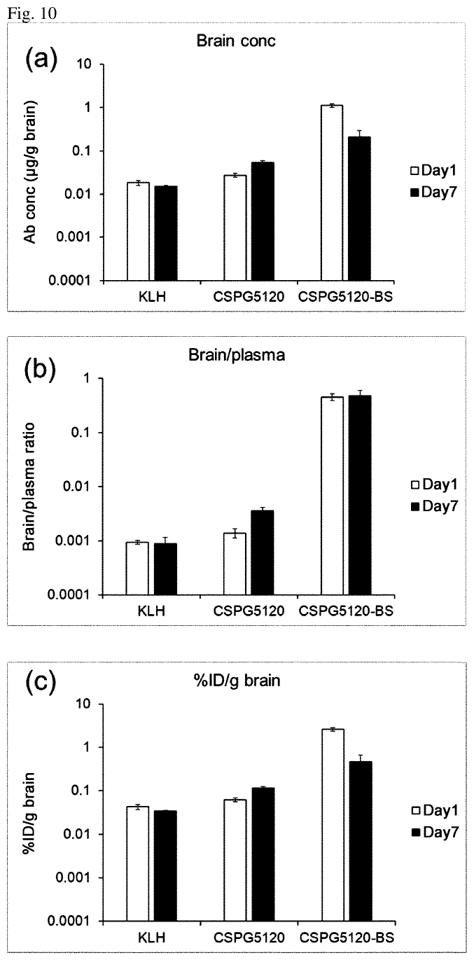

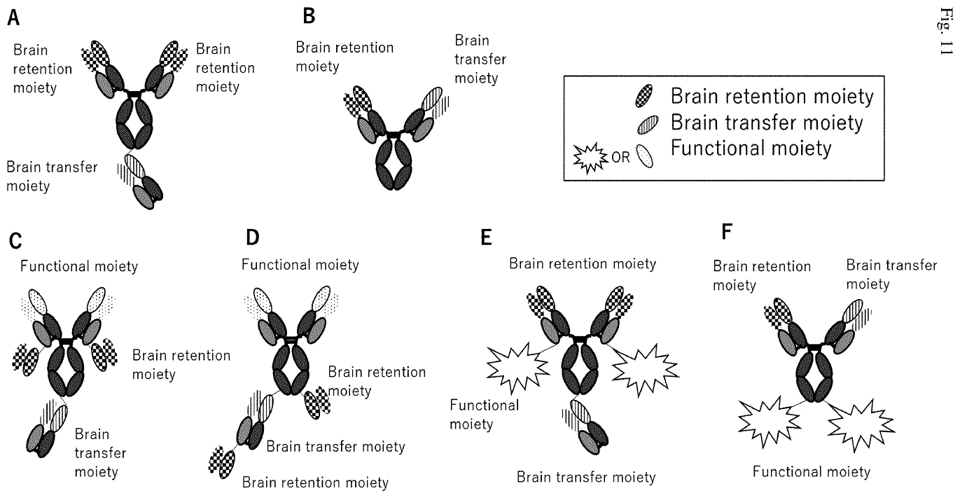

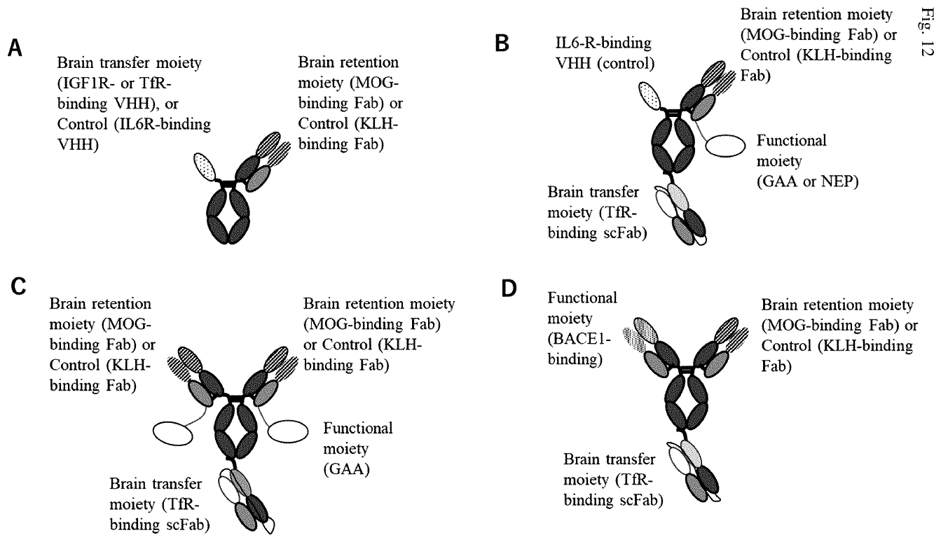

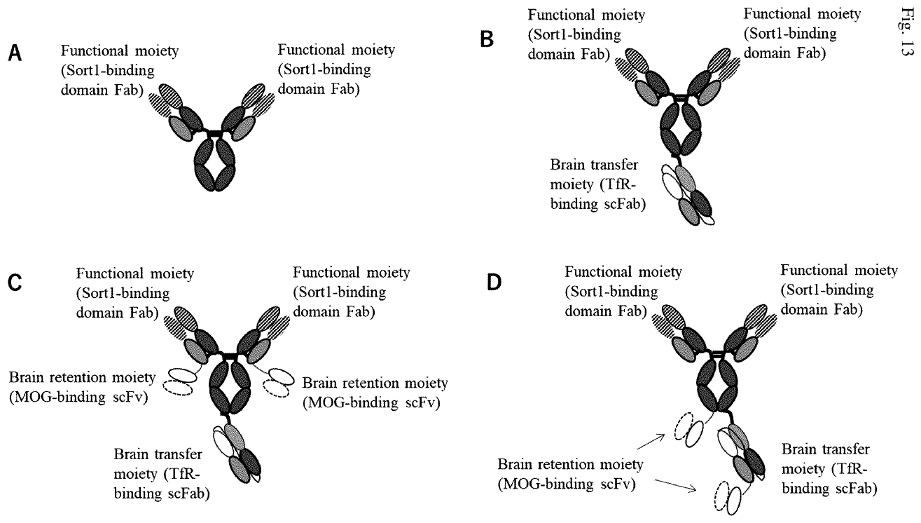

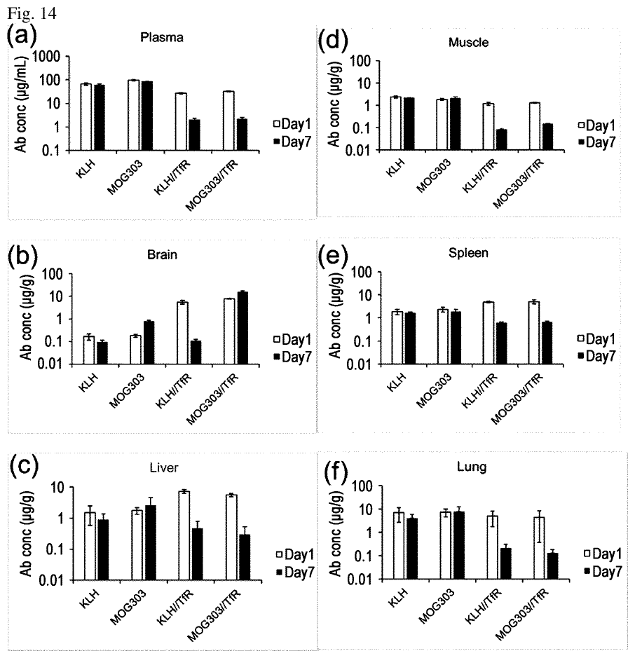

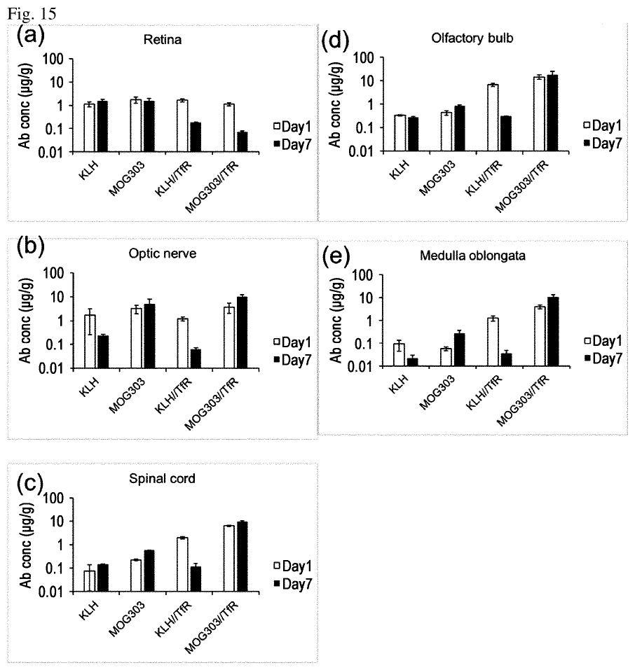

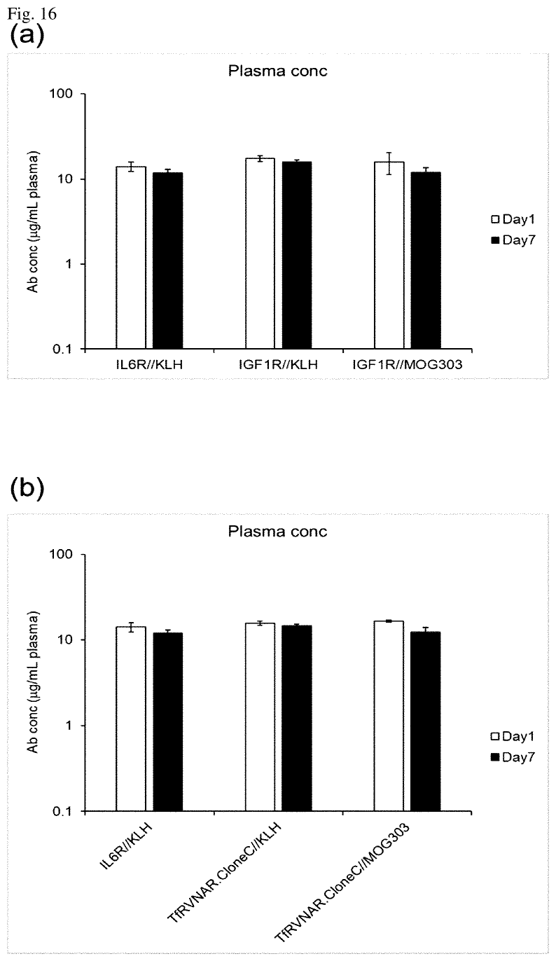

The concentrations in plasma at day 1 and day 7 were shown in FIG. 9. KLH and CSPG5120 showed similar concentrations both at day 1 and day 7. On the other hand, CSPG5120-BS showed lower concentrations than KLH and CSPG5120 at day 1 and day 7. These data suggest that the antibodies with brain transfer moiety were cleared from plasma rapidly due to the systemic expression of TfR, and the brain retention moiety, CSPG5, had minor contribution on the clearance of the antibodies.

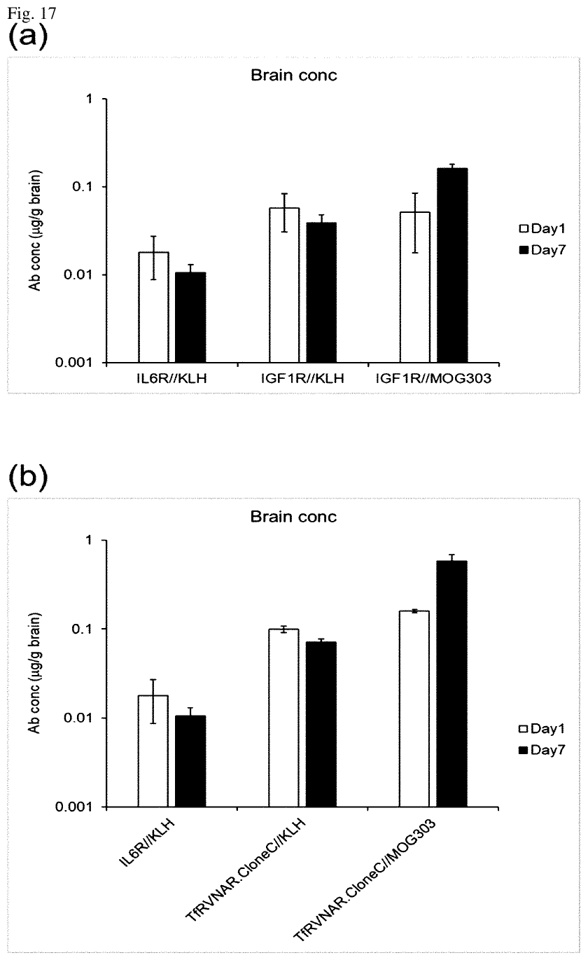

The concentrations in brain at day 1 and day 7 were shown in FIG. 10(a), (b), (c). CSPG5120 showed slightly higher concentrations at day 1 and day 7 than KLH, and the concentrations increased until day 7, suggesting the antibody accumulated in the brain. Interestingly, CSPG5120-BS showed much higher concentration at day 1 and day 7 than KLH and CSPG5120.

These results indicate that the combination of a brain transfer moiety binding to TfR and brain retention moiety binding to CSPG5 has superior potency in the transfer and retention into the brain.

### Example 8. Bispecific Antibodies Each Comprising a Brain Transfer Moiety (i.e. Anti-Transferrin Receptor Antibody or Anti-IGF1R Antibody) and a Brain Retention Moiety (i.e. Anti-MOG Antibody)

Bispecific antibodies each comprising a brain transfer moiety (i.e. anti-transferrin receptor antibody or anti-IGF1R antibody) and a brain retention moiety (i.e. anti-MOG antibody) i.e. IGF1R/MOG303 and TfRVNAR.CloneC/MOG303, or control antibodies IL6R/KLH and IGF1R/KLH as shown in Table 8 were generated:
a) IL6R/KLH (bispecific antibody of anti-hIL6R antibody and anti-KLH antibody),
b) IGF1R/KLH (bispecific antibody of anti-IGF1R antibody and anti-KLH antibody),
c) TfRVNAR.CloneC/KLH (bispecific antibody of anti-transferrin receptor antibody and anti-KLH antibody),
d) IGF1R/MOG303 (bispecific antibody of anti-mouse MOG antibody and anti-IGF1R antibody), and
e) TfRVNAR.CloneC/MOG303 (bispecific antibody of anti-mouse MOG antibody and anti-transferrin receptor antibody).

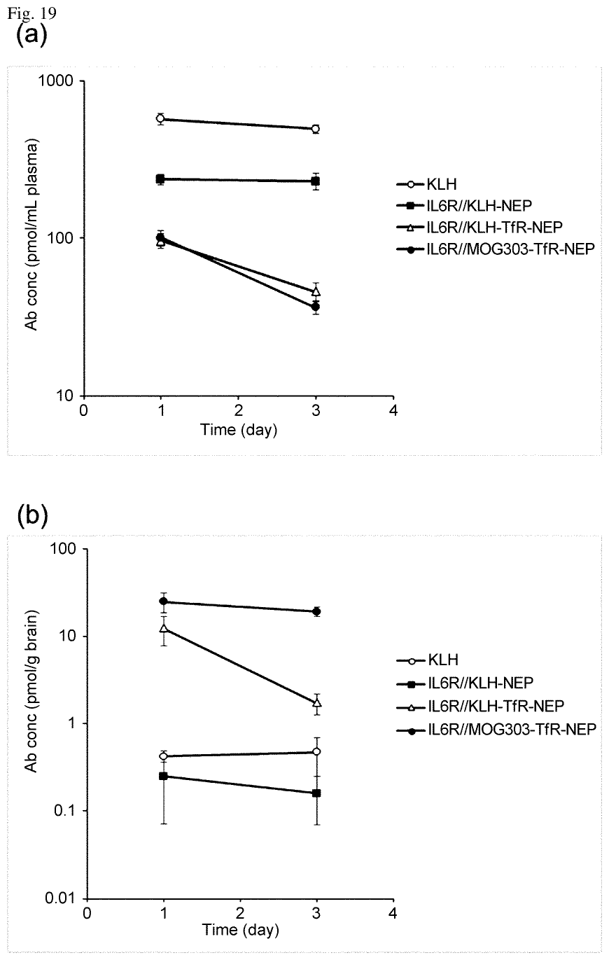

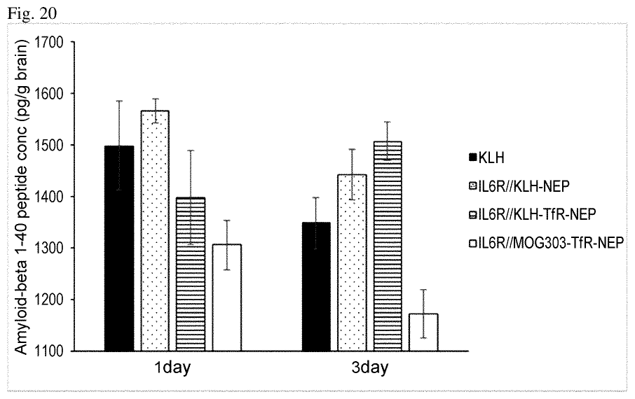

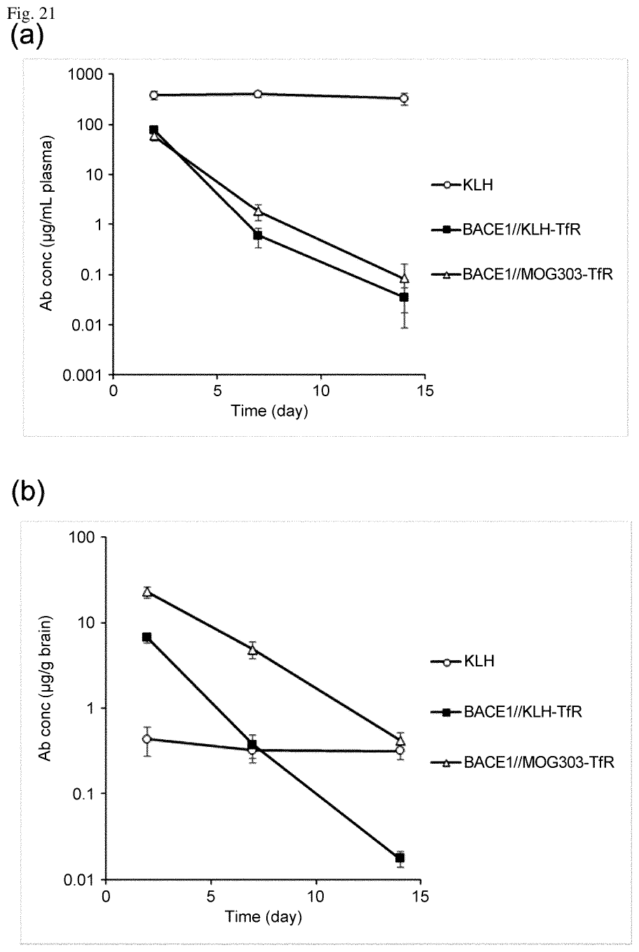

The molecular format of each of the antibodies (a) to (e) is showed in FIG. 12(A).

TABLE 8

| | Brain | | |
|---|---|---|---|
| | transfer | Retention | Retention |
| | moiety | moiety | moiety |
| Sample name | (heavy chain 1) | (heavy chain 2) | (light chain) |
| IL6R//KLH | IL6R90- | IC17HdK- | IC17L-SK1 |
| | Sal.1FcSG181Ev11h | SG181v11k | (SEQ ID NO: 74) |
| | (SEQ ID NO: 69) | (SEQ ID NO: 72) | |
| IGF1R//KLH | IGF1R5.G4S2- | IC17HdK- | IC17L-SK1 |
| | Sal.1FcSG181Ev11h | SG181v11k | (SEQ ID NO : 74) |
| | SEQ ID NO: 70) | SEQ ID NO: 72) | |
| TfRVNAR.CloneC//KLH | mTfR.VNAR.CloneC- | IC17HdK- | IC17L-SK1 |
| | Sal.1FcSG181Ev11h | SG181v11k | (SEQ ID NO: 74) |
| | SEQ ID NO: 71) | (SEQ ID NO: 72) | |
| IGF1R//MOG303 | IGF1R5.G4S2- | MOG303VH- | MOG303VL-SK1 |
| | Sal.1FcSG181Ev11h | SG181v11k | (SEQ ID NO: 75) |
| | SEQ ID NO: 70) | (SEQ ID NO: 73) | |
| TRVNAR.CloneC//MOG303 | mTfR.VNAR.CloneC- | MOG303VH- | MOG303VL-SK1 |
| | Sal.1FcSG181Ev11h | SG181v11k | (SEQ ID NO: 75) |
| | SEQ ID NO: 71) | (SEQ ID NO: 73) | |

As for the brain transfer moiety (Heavy chain 1), anti-IGF1R VHH (IGF1R, comprising H chain variable region of SEQ ID NO: 62) or anti-Transferrin receptor VHH (TfRVNAR.CloneC, comprising H chain variable region of SEQ ID NO: 63) were used, and each of the antibodies comprise heavy chain constant region of modified hIgG1 (SEQ ID NO: 64).

As for the brain retention moiety (Heavy chain 2 and Light chain), anti-MOG Fab (MOG303; H chain variable region: SEQ ID NO: 65, L chain variable region: SEQ ID NO:66 was used, and each of the antibodies comprises heavy chain constant region of modified hIgG1 (SEQ ID NO: 67) and light chain constant region of human kappa region (SEQ ID NO: 4).

As for control antibodies (i.e. non-brain transfer moiety and non-brain retention moiety), anti-KLH Fab (KLH) was used and comprises heavy chain constant region of modified hIgG1 and human kappa region; and anti-human IL6R binding VHH (H chain variable region: SEQ ID NO: 68) was used and comprises heavy chain constant region of modified hIgG1 (SEQ ID NO: 64).

Expression vectors encoding heavy chains and light chains of antibodies a) to e) were constructed by a method known in the art. All antibodies were transiently expressed in mammalian cells by the method known to those skilled in the art using the genes constructed and were purified by the method known to those skilled in the art. Expression was done by using HEK293 cell expression system. Heavy chain 1, Heavy chain 2 and Light chain were transfected to Expi293 cell according to manufacturer's protocol. After culturing of several days the cultured medium was collected and subjected to the affinity column. The antibody proteins were purified by affinity column (Protein A) and were subjected to size exclusion chromatography to obtain antibody. The expression and purification methods were conducted by a method known in the art (Nat Protoc. 2018 January; 13(1):99-117).

### Example 9. Preparation of Antibodies Having a Brain Transfer Moiety, a Brain Retention Moiety, and a Functional Moiety

### Example 9A: Acid Alpha-Glucosidase Glycosidase (GAA)-Fused Antibody

MOG303-TfR-2GAA, an antibody which comprises a transferrin receptor (TfR)-binding domain (brain transfer moiety), two myelin oligodendrocyte glycoprotein (MOG)-binding domain (brain retention moiety) and two acid alpha-glucosidase Glycosidase proteins (GAA, Uniprot accession no: P10253; functional moiety) was generated. The gene encoding GAA was fused with the 3′ of light chain constant region by a method known in the art.

MOG303-TfR-2GAA is a bispecific antibody of anti-MOG antibody and anti-Transferrin receptor antibody, and comprises two GAAs fused to the C terminal of each of the two L chains (Light chain 1 and 2) of anti-MOG antibody, i.e. two GAA within one molecule). The heavy chains and light chains sequence of the antibody are shown in Table 9. The molecular format of MOG303-TfR-2GAA is showed in FIG. 12(C).

GAA is known to be cleaved in multiple sites and one of the candidate GAA sequences to be fused to the antibody is shorter version of GAA variant which comprises amino acids 70 to 952 in the amino acid sequence set forth in Uniprot accession no: P10253. The shorter GAA variant is disclosed in the Journal of Biological Chemistry, Volume 280, Issue 8, 25 Feb. 2005, Pages 6780-6791. MOG303-TfR-2shortGAA is a bispecific antibody of anti-MOG antibody and anti-Transferrin receptor antibody, and comprises two GAAs each of which is short version of GAA comprising amino acids 70 to 952 in the amino acid sequence set forth in Uniprot accession no: P10253) fused to the C terminal of each of the two L chains (Light chain 1 and 2) of anti-MOG antibody, i.e. two short version of GAA within one molecule). The heavy chains and light chains sequence of the antibody are shown in Table 9. The molecular format of MOG303-TfR-2GAA is showed in FIG. 12(C).

Acid alpha-glucosidase (GAA), also called alpha-1,4-glucosidase and acid maltase, is an essential enzyme that helps to break down glycogen in the lysosome. Defects or deficiency in this gene are the cause of glycogen storage disease II, also known as Pompe disease. Therefore, it is expected that MOG303-TfR-2GAA or MOG303-TfR-2shortGAA could be used as therapeutics for Pompe disease that deliver a functional version of the GAA enzyme to correct the disease particularly in the CNS.

In addition, the following control antibodies were generated, of which their heavy chains and light chains sequences are shown in Table 9.
a) IL6R/KLH-GAA (bispecific antibody of anti-hIL6R antibody and anti-KLH antibody, and comprises one GAA fused to the C terminal of L chain of anti-KLH antibody, i.e. IL6R/KLH-GAA comprises one GAA),
b) IL6R/KLH-TfR-GAA (multi-specific antibody of anti-hIL6R antibody, anti-KLH antibody and anti-Transferrin receptor antibody, comprising one GAA fused to the C terminal of L chain of anti-KLH antibody, i.e. IL6R/KLH-TfR-GAA comprises one GAA),
c) IL6R/MOG303-TfR-GAA (multi-specific antibody of anti-hIL6R antibody, anti-MOG antibody and anti-transferrin receptor antibody, comprising one GAA fused to C terminal of L chain of anti-KLH antibody, i.e. IL6R/MOG303-TfR-GAA comprises one GAA),
d) KLH-2GAA (one GAA fused to the C terminal of each of the two L chains of bivalent anti-KLH antibody, i.e. KLH-2GAA comprises two GAAs), and
e) KLH-TfR-2GAA (bispecific antibody of anti-KLH antibody and anti-Transferrin receptor antibody, comprising one GAA fused to the C terminal of each of the two L chains of anti-KLH antibody, i.e. KLH-TfR-2GAA comprises two GAAs).
f) IL6R/KLH-shortGAA (bispecific antibody of anti-hIL6R antibody and anti-KLH antibody, and comprises one short version of GAA fused to the C terminal of L chain of anti-KLH antibody, i.e. IL6R/KLH-shortGAA comprises one short version of GAA),
g) IL6R/KLH-TfR-shortGAA (multi-specific antibody of anti-hIL6R antibody, anti-KLH antibody and anti-Transferrin receptor antibody, comprising one short version of GAA fused to the C terminal of L chain of anti-KLH antibody, i.e. IL6R/KLH-TfR-shortGAA comprises one short version of GAA),
h) IL6R/MOG303-TfR-shortGAA (multi-specific antibody of anti-hIL6R antibody, anti-MOG antibody and anti-transferrin receptor antibody, comprising one short version of GAA fused to C terminal of L chain of anti-KLH antibody, i.e. IL6R/MOG303-TfR-GAA comprises one short version of GAA),
i) KLH-2shortGAA (one short version of GAA fused to the C terminal of each of the two L chains of bivalent anti-KLH antibody, i.e. KLH-2shortGAA comprises two short version of GAAs), and
j) KLH-TfR-2shortGAA (bispecific antibody of anti-KLH antibody and anti-Transferrin receptor antibody, comprising one short version of GAA fused to the C terminal of each of the two L chains of anti-KLH antibody, i.e. KLH-TfR-2short GAA comprises two short version of GAAs).

The molecular format of IL6R/KLH-GAA, IL6R/KLH-TfR-GAA, IL6R/MOG303-TfR-GAA, IL6R/KLH-shortGAA, IL6R/KLH-TfR-shortGAA and IL6R/MOG303-TfR-shortGAA is showed in FIG. 12(B), whereas the molecular format of KLH-2GAA, KLH-TfR-2GAA, MOG303-TfR-2GAA, KLH-2shortGAA, KLH-TfR-2shortGAA and MOG303-TfR-2shortGAA is showed in FIG. 12(C).

Expression vectors encoding heavy chains and light chains of IL6R/KLH-GAA, IL6R/KLH-TfR-GAA, IL6R/MOG303-TfR-GAA, KLH-2GAA, KLH-TfR-2GAA and MOG303-TfR-2GAA as shown in Table 9 were constructed by a method known in the art.

Expression vectors encoding heavy chains and light chains of IL6R/KLH-shortGAA, IL6R/KLH-TfR-shortGAA, IL6R/MOG303-TfR-shortGAA, KLH-2shortGAA, KLH-TfR-2shortGAA and MOG303-TfR-2shortGAA as shown in Table 9 are constructed by a method known in the art.

TABLE 9

| | | | Light chain 1 |
|---|---|---|---|
| Antibody | Heavy chain 1 | Heavy chain 2 | and/or 2 |
| IL6R//KLH- | IC17HdK-SG181v11k | IL6R90-nSG181v11h | IC17L-SK1.hGAA |
| GAA | (SEQ ID NO: 72) | (SEQ ID NO: 79) | (SEQ ID NO: 82) |
| IL6R//KLH- | IC17HdK- | IL6R90-nSG181v11h | ICI7L-SK1.hGAA |
| TfR-GAA | SG181v11k.newmBBB | (SEQ ID NO: 79) | (SEQ ID NO: 82) |
| | (SEQ ID NO: 76) | | |
| IL6R//MOG303- | MOG303VH- | IL6R90-nSG181v11h | MOG303VL- |
| TfR-GAA | SG181v11k.newmBBB | (SEQ ID NO: 79) | SK1.hGAA |
| | (SEQ ID NO: 77) | | (SEQ ID NO: 83) |
| KLH-2GAA | IC17HdK-SG181 | IC17HdK-SG181 | IC17L-SK1.hGAA |
| | (SEQ ID NO: 78) | (SEQ ID NO: 78) | (SEQ ID NO: 82) |
| KLH-TfR- | IC17HdK- | IC17HdK-SG181v11h | IC17L-SK1.hGAA |
| 2GAA | SG181v11k.newmBBB | (SEQ ID NO: 80) | (SEQ ID NO: 82) |
| | (SEQ ID NO: 76) | | |
| MOG303-TfR- | MOG303VH- | MOG303VH- | MOG303VL- |
| 2GAA | SG181v11k.newmBBB | SG181v11h | SK1.hGAA |
| | (SEQ ID NO: 77) | (SEQ ID NO: 81) | (SEQ ID NO: 83) |
| IL6R//KLH- | IC17HdK-SG181v11k | IL6R90-nSG181v11h | IC17L- |
| shortGAA | (SEQ ID NO: 72) | (SEQ ID NO: 79) | SK1.hGAA.short |
| | | | (SEQ ID NO: 98) |
| IL6R//KLH- | IC17HdK- | IL6R90-nSG181v11h | IC17L- |
| TfR-shortGAA | SG181v11k.newmBBB | (SEQ ID NO: 79) | SK1.hGAA.short |
| | (SEQ ID NO: 76) | | (SEQ ID NO: 98) |
| IL6R//MOG303- | MOG303VH- | IL6R90-nSG181v11h | MOG303VL- |
| TfR-shortGAA | SG181v11k.newmBBB | (SEQ ID NO: 79) | SK1.hGAA.short |
| | (SEQ ID NO: 77) | | (SEQ ID NO: 99) |
| KLH- | IC17HdK-SG181 | IC17HdK-SG181 | IC17L- |
| 2shortGAA | (SEQ ID NO: 78) | (SEQ ID NO: 78) | SK1.hGAA.short |
| | | | (SEQ ID NO: 98) |
| KLH-TfR- | IC17HdK- | IC17HdK-SG181v11h | IC17L- |
| 2shortGAA | SG181v11k.newmBBB | (SEQ ID NO: 80) | SK1.hGAA.short |
| | (SEQ ID NO: 76) | | (SEQ ID NO: 98) |
| MOG303-TfR- | MOG303VH- | MOG303VH- | MOG303VL- |
| 2shortGAA | SG181v11k.newmBBB | SG181v11h | SK1.hGAA.short |
| | (SEQ ID NO: 77) | (SEQ ID NO: 81) | (SEQ ID NO: 99) |

Expression of IL6R/KLH-GAA, IL6R/KLH-TfR-GAA, IL6R/MOG303-TfR-GAA, KLH-2GAA, KLH-TfR-2GAA and MOG303-TfR-2GAA as shown in Table 9 was done by using HEK293 cell expression system. Heavy chain 1, Heavy chain 2 and Light chain(s) were transfected to Expi293 cell according to manufacturer's protocol. After culturing of several days the cultured medium was collected and subjected to the affinity column. The antibody proteins were purified by affinity column (Protein A) and were subjected to size exclusion chromatography to obtain protein of interest. The expression and purification methods were conducted by a method known in the art (Nat Protoc. 2018 January; 13(1):99-117).

Expression of IL6R/KLH-shortGAA, IL6R/KLH-TfR-shortGAA, IL6R/MOG303-TfR-shortGAA, KLH-2shortGAA, KLH-TfR-2shortGAA and MOG303-TfR-2shortGAA as shown in Table 9 is done by using HEK293 cell expression system. Heavy chain 1, Heavy chain 2 and Light chain(s) are transfected to Expi293 cell according to manufacturer's protocol. After culturing of several days the cultured medium is collected and subjected to the affinity column. The antibody proteins are to be purified by affinity column (Protein A) and are subjected to size exclusion chromatography to obtain protein of interest. The expression and purification methods are conducted by a method known in the art (Nat Protoc. 2018 January; 13(1):99-117).

### Example 9B: Neprilysin (NEP)-Fused Antibody

IL6R/MOG303-TfR-NEP, a multispecific antibody which comprises a transferrin receptor (TfR)-binding domain (brain transfer moiety), a Myelin oligodendrocyte Glycoprotein (MOG)-binding domain (brain retention moiety), an anti-hIL6R antibody, and one Neprilysin (NEP, Uniprot accession no: P08473) protein was generated as described below. Neprilysin (NEP) is a membrane-bound metallopeptidase and one of the major A beta-degrading enzymes, and has been previously reported as a potential protein-therapy degrading A beta in Alzheimer's disease.

IL6R/MOG303-TfR-NEP comprises one NEP is fused to the C terminal of L chain of anti-MOG antibody (i.e. the antibody comprises one NEP). The gene encoding NEP was fused with the 3′ of light chain constant region by a method known in the art. The heavy chains and light chains sequence of the antibody are shown in Table 10. The molecular format of IL6R/MOG303-TfR-NEP is showed in FIG. 12(B).

In addition, the following control antibodies were generated, of which their heavy chains and light chain sequences are shown in Table 10 and their molecular format is shown in FIG. 12(B):
a) IL6R/KLH-NEP (antibody of anti-hIL6R antibody and H chain of anti-KLH antibody and L chain of anti-MOG antibody, comprising one NEP fused to the C terminal of L chain of anti-MOG antibody, i.e. IL6R/KLH-NEP comprises one NEP), and;
b) IL6R/KLH-TfR-NEP (multispecific antibody of anti-hIL6R antibody, H chain of anti-KLH antibody with L chain of anti-MOG antibody and anti-Transferrin receptor antibody, comprising one NEP fused to the C terminal of L chain of anti-MOG antibody, IL6R/KLH-TfR-NEP comprises one NEP).

Expression vectors encoding heavy chains 1 and 2 and light chain of IL6R/KLH-NEP, IL6R/KLH-TfR-NEP and IL6R/MOG303-TfR-NEP as shown in Table 10 were constructed by a method known in the art.

TABLE 10

| Antibody | Heavy chain 1 | Heavy chain 2 | Light chain |
|---|---|---|---|
| IL6R//KLH- | IC17HdK-SG181v11k | IL6R90-nSG181v11h | MOG303VL- |
| NEP | (SEQ ID NO: 72) | (SEQ ID NO: 79) | SK1.N02.hNEP |
| | | | (SEQ ID NO: 84) |
| IL6R//KLH- | IC17HdK- | IL6R90-nSG181v11h | MOG303VL- |
| TfR-NEP | SG181v11k.newmBBB | (SEQ ID NO: 79) | SK1.N02.hNEP |
| | (SEQ ID NO: 76) | | (SEQ ID NO: 84) |
| IL6R//MOG303- | MOG303VH- | IL6R90-nSG181v11h | MOG303VL- |
| TfR-NEP | SG181v11k.newmBBB | (SEQ ID NO: 79) | SK1.N02.hNEP |
| | (SEQ ID NO: 77) | | (SEQ ID NO: 84) |

Expression of IL6R/KLH-NEP, IL6R/KLH-TfR-NEP, IL6R/MOG303-TfR-NEP as shown in Table 10 was done by using HEK293 cell expression system. Heavy chain 1, Heavy chain 2 and Light chain were transfected to Expi293 cell according to manufacturer's protocol. After culturing of several days the cultured medium was collected and subjected to the affinity column. The antibody proteins were purified by affinity column (Protein A) and were subjected to size exclusion chromatography to obtain protein of interest. The expression and purification methods were conducted by a method known in the art (Nat Protoc. 2018 January; 13(1):99-117).

### Example 9C: Antibody Comprising Anti-BACE1 Binding Domain as Functional Moiety

BACE1/MOG303-TfR, a multispecific antibody which comprises a transferrin receptor (TfR)-binding domain (brain transfer moiety), Myelin oligodendrocyte Glycoprotein (MOG)-binding domain (brain retention moiety) and a BACE1 binding domain (functional moiety) was generated as described below. BACE1 is an enzyme essential for the generation of beta-amyloid and antibodies that inhibit BACE1 could reduce beta-amyloid and its associated toxicities. The heavy chains and light chains sequence of the BACE1/MOG303-TfR are shown in Table 11 and its molecular format is showed in FIG. 12(D).

As control antibody, BACE1/KLH-TfR (bispecific antibody of anti-BACE1 antibody and anti-Transferrin receptor antibody) was also generated. The heavy chains and light chains sequence of the antibody are shown in Table 11 and its molecular format is showed in FIG. 12(D).

TABLE 11

| Antibody | Heavy chain 1 | Heavy chain 2 | Light chain 1 | Light chain 2 |
|---|---|---|---|---|
| BACE1//KLH- | BACE1.6266VH- | IC17L- | BACE1.6266VL- | IC17HdK-xSK1 |
| TfR | SG181009v11h | xSG181v11k. | RKRohuK1 | (SEQ ID NO: 89) |
| | (SEQ ID NO: 85) | newmBBB | (SEQ ID NO: 88) | |
| | | (SEQ ID NO: 86) | | |
| BACE1// | BACE1.6266VH- | MOG303VL | BACE1.6266VL- | MOG303VH- |
| MOG303-TfR | SG181009v11h | xSG181v11k. | RKRobuK1 | xSK1 |
| | (SEQ ID NO: 85) | newmBBB | (SEQ ID NO: 88) | (SEQ ID NO: 90) |
| | | (SEQ ID NO: 87) | | |

Expression vectors encoding heavy chains and light chains of BACE1/KLH-TfR, BACE1/MOG303-TfR as shown in Table 11 were constructed by a method known in the art. Expression of BACE1/KLH-TfR, BACE1/MOG303-TfR as shown in Table Z was done by using HEK293 cell expression system. Heavy chain 1, Heavy chain 2, Light chain 1 and Light chain 2 were transfected to Expi293 cell according to manufacturer's protocol. After culturing of several days the cultured medium was collected and subjected to the affinity column. The antibody proteins were purified by affinity column (Protein A) and were subjected to size exclusion chromatography to obtain protein of interest. The expression and purification methods were conducted by a method known in the art (Nat Protoc. 2018 January; 13(1):99-117).

### Example 9D: Antibody Comprising Anti-SORT1 Binding Domain as Functional Moiety

The following antibodies which comprise a transferrin receptor (TfR)-binding domain (brain transfer moiety), Myelin oligodendrocyte Glycoprotein (MOG)-binding domain (brain retention moiety) and a SORT1 binding domain (functional moiety) was generated as described below:
a) Sort1-TfR-MOG303(L): a multispecific antibody of anti-SORT1 antibody, anti-Transferrin receptor antibody and anti-MOG scFv linked at the C terminal of L chain of anti-SORT1 antibody), which has a molecular format shown in FIG. 13(C); and
b) Sort1-TfR-MOG303(H): a multispecific antibody of anti-SORT1 antibody, anti-Transferrin receptor antibody and anti-MOG scFv linked at the C terminal of H chain, which has a molecular format shown in FIG. 13(D).

SORT1 is a transmembrane receptor that controls the extracellular level of progranulin (PGRN) by binding it at the cell surface and rapidly internalizing it for lysosomal degradation. Anti-SORT1 antibodies that bind SORT1, block the interaction with PGRN, and thus functionally elevate PGRN levels have been shown to have therapeutic efficacy for the treatment of certain neurodegenerative disorders such as Frontotemporal dementia (FTD).

In Addition, the Following Control Antibodies were Generated:
c) Sort1 is a bivalent anti-Sortilin1 antibody which has a molecular format shown in FIG. 13(A); and
d) Sort1-TfR is a bispecific antibody of anti-SORT1 antibody and anti-Transferrin receptor antibody which has a molecular format shown in FIG. 13(B).

The heavy chains and light chains sequence of Sort1, Sort1-TfR, Sort1-TfR-MOG303(L) and Sort1-TfR-MOG303(H) are shown in Table 12.

TABLE 12

| Antibody | Heavy chain 1 | Heavy chain 2 | Light chain 1 & 2 |
|---|---|---|---|
| Sort1 | AXL101VH-SG181 | AXL101VH-SG181 | AXL101VL-SK1 |
| | (SEQ ID NO: 91) | (SEQ ID NO: 91) | (SEQ ID NO: 96) |
| Sort1-TfR | AXL101VH- | AXL101VH-SG181v11h | AXL101VL-SK1 |
| | SG181v11k.newmBBB | (SEQ ID NO: 94) | (SEQ ID NO: 96) |
| | (SEQ ID NO: 92) | | |
| Sort1-TfR- | AXL101VH- | AXL101VH-SG181v11h | AXL101VL- |
| MOG303(L) | SG181v11k.newmBBB | (SEQ ID NO: 94) | SK1.N14.MOG303scFv. |
| | (SEQ ID NO: 92) | | Cys |
| | | | (SEQ ID NO: 97) |
| Sort1-TfR- | AXL101VH- | AXL101VH- | AXL101VL-SK1 |
| MOG303(H) | SG181v11k.newmBBB. | SG181v11h.N14. | (SEQ ID NO: 96) |
| | N14. MOG303scFv | MOG303scFv | |
| | (SEQ ID NO: 93) | (SEQ ID NO: 95) | |

Expression vectors encoding heavy chains and light chains of Sort1, Sort1-TfR, Sort1-TfR-MOG303(L) and Sort1-TfR-MOG303(H) as shown in Table 12 were constructed by a method known in the art. Expression of Sort1, Sort1-TfR, Sort1-TfR-MOG303(L) and Sort1-TfR-MOG303(H) was done by using HEK293 cell expression system. Heavy chain 1, Heavy chain 2 and Light chain were transfected to Expi293 cell according to manufacturer's protocol. After culturing of several days the cultured medium was collected and subjected to the affinity column. The antibody proteins were purified by affinity column (Protein A) and were subjected to size exclusion chromatography to obtain protein of interest. The expression and purification methods were conducted by a method known in the art (Nat Protoc. 2018 January; 13(1):99-117).

### Example 10. Tissue Distribution of Antibodies Having Brain Transfer Moiety and Brain Retention Moiety in Certain Central Nerve Tissues

Central nerve system (CNS) tissues such as optic nerve and spinal cord have oligodendrocytes expressing MOG around neurons. On the other hand, peripheral nerve tissues (PNS) such as liver and lung have schwann cells around peripheral neurons instead of oligodendrocytes. Therefore, antibodies with brain retention moiety that binds MOG are expected to have a tendency to distribute or stay in the CNS tissues that express MOG such as optic nerve and spinal cord.

Tissue distribution of the following antibodies in plasma, brain, liver, muscle, spleen, lung, retina, optic nerve, spinal cord, olfactory bulb and medulla oblongata was evaluated in C57BL/6J mice (male, 6-8 weeks) to reveal their transferability into and retention in these tissues. KLH, the negative control antibody, MOG303, the antibody with the retention moiety, KLH/TfR, the antibody with the transfer moiety, and MOG303/TfR, the antibody with both moieties were administered intravenously in mice at a dose of 10 mg/kg. Blood was collected on day 1 and day 7 after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. A part of the pieces of minced tissues was dissolved by Cell extraction buffer (Invitrogen) containing Complete Protease Inhibitor Cocktail (Roche) in a tube with 5 mm stainless beads, and was homogenized with a homogenizer (Qiagen). Then, the samples were centrifuged (15,000 rpm, 4 degrees C., 20 min) and the supernatant was collected.

Concentrations of the antibodies in plasma and tissues were determined by the electro chemiluminescence-immunoassay (ECL). As a capture antibody, an anti-human IgG Fab′2 antibody (LifeSpan BioSciences) was applied in the plate. After PBS with Tween20 (PBST, Sigma-Aldrich) containing 1% bovine serum albumin (Sigma-Aldrich) was added as blocking solution, diluted plasma or tissue homogenate samples were applied. As a detection antibody, a biotin-labeled anti-human IgG antibody (Bethyl Laboratories) was added. Finally, a streptavidin labeled with SULFO-tag (Meso Scale Discovery) was added, and signals were detected by ECL (Meso Scale Discovery).

The concentrations in these tissues at day 1 and day 7 were shown in FIGS. 14 and 15. As shown in FIG. 14(a), in plasma, the concentration of KLH and MOG303 decreased a little until day 7, however KLH/TfR and MOG303/TfR decreased largely until day 7. This result indicates that binding to TfR had a major contribution on antibody elimination from plasma. In brain (FIG. 14(b)), time-dependent accumulation of MOG303 and MOG303/TfR was demonstrated, although KLH/TfR showed fast disappearance from brain. It suggests that binding to MOG contributes to the retention of the antibody. In the peripheral tissues such as liver (FIG. 14(c)), muscle (FIG. 14(d)), spleen (FIG. 14(e)) and lung (FIG. 14(f)), no accumulation of the antibody until day 7 was observed. Especially in liver and spleen, higher concentration of KLH/TfR and MOG303/TfR than KLH and MOG303 at day 1 was shown, however the concentration largely decreased at day 7. These data suggest that the antibodies with the retention moiety could not bind to the MOG and not stay in the brain due to no MOG-expressing oligodendrocyte in these peripheral nerve tissues. In retina (FIG. 15(a)) where oligodendrocyte does not exist in mouse and human, no retention of the antibody until day 7 was revealed. On the other hand, MOG303 accumulated in the central nerve tissues such as optic nerve (FIG. 15(b)), spinal cord (FIG. 15(c)), olfactory bulb (FIG. 15(d)) and medulla oblongata (FIG. 15(e)) until day 7, although KLH and KLH/TfR decreased until day 7. Interestingly, MOG303/TfR showed higher concentrations than other antibodies even at day 1 and accumulation until day 7. These results indicate that the antibodies with the retention moiety binds to MOG and retained in the tissues, and combination of the transfer and retention moiety have synergistic effect on the antibody concentration from day 1 in these CNS tissues.

### Example 11. Pharmacokinetic Profiles of Bispecific Antibody IGF1R/MOG303 and TfR VNAR/MOG303 in Brain and in Plasma in Mice

The concentrations in plasma and brain of the following antibodies described in Example 8 were evaluated after intravenous dosing on C57BL/6J mice (male, 6-8 weeks) to reveal their elimination from plasma, and transferability into and retention of them in brain.

IL6R/KLH (negative control antibody), IGF1R/KLH (antibody having anti-IGF1R antibody as brain transfer moiety), TfRVNAR.CloneC/KLH (antibody having anti-TfR antibody as brain transfer moiety), IGF1R/MOG303 (bispecific antibody having anti-MOG antibody as brain retention moiety and anti-IGF1R antibody as brain transfer moiety) and TfRVNAR.CloneC/MOG303 (bispecific antibody having anti-MOG antibody as brain retention moiety and anti-TfR antibody as brain transfer moiety) were administered intravenously in mice at a dose of 2 mg/kg. Blood was collected on day 1 and day 7 after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. A half of the brain was dissolved by Cell extraction buffer (Invitrogen) containing Complete Protease Inhibitor Cocktail (Roche) in M tube (Miltenyi), and was homogenized with gentle MACS (Miltenyi). Homogenate was centrifuged (15,000 rpm, 4 degrees C., 20 min) and the supernatant was collected.

Concentrations of the antibodies in plasma and brain were determined by the electro chemiluminescence-immunoassay (ECL). As a capture antibody, an anti-human IgG Fab′2 antibody (LifeSpan BioSciences) was applied in the plate. After PBS with Tween20 (PBST, Sigma-Aldrich) containing 1% bovine serum albumin (Sigma-Aldrich) was added as blocking solution, diluted plasma or brain homogenate samples were applied. As a detection antibody, a biotin-labeled anti-human IgG antibody (Bethyl Laboratories) was added. Finally, a streptavidin labeled with SULFO-tag (Meso Scale Discovery) was added, and signals were detected by ECL (Meso Scale Discovery).

The concentrations of the antibodies in plasma at day 1 and day 7 were shown in FIG. 16. As shown in FIG. 16(a), IGF1R/KLH and IGF1R/MOG303 showed similar concentrations with IL6R/KLH. Similarly, TfRVNAR.CloneC/KLH and TfRVNAR.CloneC/MOG303 showed comparable concentrations in plasma with IL6R/KLH (FIG. 16(b)). These results suggest that the antibodies with these transfer moieties such as IGF1R and TfRVNAR.CloneC had minor contribution on the clearance of the antibodies.

The concentrations of the antibodies in brain at day 1 and day 7 were shown in FIG. 17. As shown in FIG. 17(a), IL6R/KLH showed around 0.02 microgram/g brain at day 1, and decreased until day 7. The concentrations of IGF1R/KLH in brain were higher than IL6R/KLH at day 1 and 7, respectively (FIG. 17(a)). Similarly, TfRVNAR.CloneC showed higher concentrations than IL6R/KLH (FIG. 17(b)) in brain. Interestingly, IGF1R/MOG303 and TfRVNAR.CloneC/MOG303 showed similar or higher concentration in brain at day 1 than IGF1R/KLH and TfRVNAR.CloneC/KLH, respectively. At day 7, the concentrations of IGF1R/MOG303 and TfRVNAR.CloneC/MOG303 in brain increased highly compared to IGF1R/KLH and TfRVNAR.CloneC/KLH (FIG. 17(a) and 17(b)). These data suggest that the combination of the brain transfer moiety and brain retention moiety has synergistic effect which allows high exposure of an antibody having both brain transfer moiety and brain retention moiety in brain, compared to antibodies having brain transfer moiety or brain retention moiety alone.

### Example 12. Concentration in Plasma and in Brain of the Antibodies Having a Brain Transfer Moiety, a Brain Retention Moiety, and a Functional Moiety

### Example 12A: Antibody Comprising Anti-SORT1 Binding Domain as Functional Moiety

The concentrations in plasma and brain of the following antibodies described in Example 9D were evaluated after intravenous dosing on C57BL/6J mice (male, 6-8 weeks) to reveal their elimination from plasma, transferability into and retention of them in brain.

KLH (negative control antibody), Sort1 (the anti-Sortilin1 antibody), Sort1-TfR (the anti-Sortilin1 antibody having anti-TfR antibody as brain transfer moiety), and Sort1-TfR-MOG303(L) and Sort1-TfR-MOG303(H) (the anti-Sortilin1 antibodies having the brain transfer moiety, and brain retention moiety linked to the light chain and heavy chain respectively) were administered intravenously in mice at a dose of 200 nmol/kg. Blood was collected on day 1 and day 7 after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. A half of the brain was dissolved by Cell extraction buffer (Invitrogen) containing Complete Protease Inhibitor Cocktail (Roche) in M tube (Miltenyi), and was homogenized with gentle MACS (Miltenyi). Homogenate was centrifuged (15,000 rpm, 4 degrees C., 20 min) and the supernatant was collected.

Concentrations of the antibodies in plasma and brain were determined by the electro chemiluminescence-immunoassay (ECL). As a capture antibody, an anti-human IgG Fab′2 antibody (LifeSpan BioSciences) was applied in the plate. After PBS with Tween20 (PBST, Sigma-Aldrich) containing 1% bovine serum albumin (Sigma-Aldrich) was added as blocking solution, diluted plasma or brain homogenate samples were applied. As a detection antibody, a biotin-labeled anti-human IgG antibody (Bethyl Laboratories) was added. Finally, a streptavidin labeled with SULFO-tag (Meso Scale Discovery) was added, and signals were detected by ECL (Meso Scale Discovery).

The concentrations of the antibodies in plasma at day 1 and day 7 were shown in FIG. 18(a). Antibody Sort1 showed lower concentration in plasma at day 1 and day 7 compared to KLH. For Sort1-TfR, significant reduction of the antibody concentrations in plasma was found at day 7, and similar antibody concentration in plasma was also observed in Sort1-TfR-MOG303(L) and Sort1-TfR-MOG303(H). These results suggest that binding to Sortilin1 and TfR, but not MOG have an influence on antibody PK (faster clearance) in plasma.

The concentrations of the antibodies in brain at day 1 and day 7 were shown in FIG. 18(b). Sort1 showed comparable antibody concentration in brain with KLH at day 1, and lower concentration in brain than KLH at day 7. For Sort1-TfR, Sort1-TfR-MOG303(L), and Sort1-TfR-MOG303(H), significantly higher concentrations were found than Sort1 at day 1. At day 7, the concentration of Sort1-TfR in brain was lower than that of Sort1. Interestingly, the concentrations of Sort1-TfR-MOG303(L) and Sort1-TfR-MOG303(H) in brain at day 7 were higher than that of antibodies Sort1 and Sort1-TfR. These data suggest that the combination of the brain transfer moiety and brain retention moiety has a contribution to high exposure of an anti-Sortilin1 antibody in brain.

### Example 12B: Pharmacokinetic Profiles and Pharmacodynamic Effect of an Antibody Conjugated with NEP in Plasma and in Brain

Pharmacokinetics of IgG antibodies of anti-KLH-IgG (KLH), anti-KLH-IgG conjugated with neprilysin (NEP) (IL6R/KLH-NEP), anti-KLH-IgG conjugated with anti-TfR domain and NEP (IL6R/KLH-TfR-NEP) and anti-MOG303-IgG conjugated with anti-TfR domain and NEP (IL6R/MOG303-TfR-NEP) described in Example 9B were evaluated on C57BL/6J mice (male, 7 weeks) to reveal their elimination from plasma, transferability into and retention in brain.

Antibodies KLH, IL6R/KLH-NEP, IL6R/KLH-TfR-NEP and IL6R/MOG303-TfR-NEP were administered intravenously in mice at a dose of 50 nmol/kg. Blood was collected at 1 day and 3 day after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. Hemispheres of mouse whole brain were dissolved by cell extraction buffer (Invitrogen) containing Complete Mini Protease Inhibitor Cocktail (Roche) in M tube (Miltenyi Biotec), and homogenized with gentleMACS Dissociator (Miltenyi Biotec). Homogenates were centrifuged (15,000 rpm, 4 degrees C., 20 min) and the supernatants were collected.

Concentrations of the antibodies in plasma and in brain were determined by the electro chemiluminescence-immunoassay (ECL). As a capture antibody, an anti-human IgG Fab′2 antibody (LifeSpan BioSciences) was applied in the plate. After PBS with Tween20 (PBST, Sigma-Aldrich) containing 1% bovine serum albumin (Sigma-Aldrich) was added as blocking solution, diluted plasma and brain homogenate samples were applied. As a detection antibody, a biotin-labeled anti-human IgG antibody (Bethyl Laboratories) was added. Finally, a streptavidin labeled with SULFO-tag (Meso Scale Discovery) was added, and signals were detected by ECL (Meso Scale Discovery).

The pharmacokinetic profiles of the antibodies in plasma were shown in FIG. 19(a). IL6R/KLH-TfR-NEP and IL6R/MOG303-TfR-NEP showed lower concentrations at day 1, 3 and were eliminated much faster than KLH and IL6R/KLH-NEP. The result suggests that antibodies with anti-TfR domain were eliminated from plasma rapidly due to the systemic expression of TfR.

The pharmacokinetic profiles of the antibodies in brain were shown in FIG. 19(b). IL6R/KLH-TfR-NEP and IL6R/MOG303-TfR-NEP showed higher concentrations at day 1 and day 3, compared with KLH and IL6R/KLH-NEP. In addition, IL6R/MOG303-TfR-NEP showed higher concentration compared with IL6R/KLH-TfR-NEP at day 1 and day 3. These results indicate that combination of the brain transfer moiety (anti-TfR) and the brain retention moiety (anti-MOG) have synergistic effect on the potency of the antibody transfer and retention into the brain.

In addition, pharmacodynamic effect of KLH, IL6R/KLH-NEP, IL6R/KLH-TfR-NEP and IL6R/MOG303-TfR-NEP antibodies were monitored to study the changes of amyloid-beta 1-40 peptide concentration in brain as a result of the degradation by NEP enzyme (an A beta-degrading enzymes) that is conjugated with the antibodies.

KLH, IL6R/KLH-NEP, IL6R/KLH-TfR-NEP and IL6R/MOG303-TfR-NEP were administered intravenously in mice at a dose of 50 nmol/kg. At 1 day and 3 days after antibody administration, PBS was perfused from the heart by inserting a catheter to remove blood, and then brain was collected. A piece of mouse whole brain was dissolved by 5 M Guanidine (Wako) and homogenized with stainless beads (Qiagen) by TissueLyser (Qiagen). Homogenates were diluted with 1% BSA-PBST containing Complete Mini Protease Inhibitor Cocktail (Roche) and the supernatants were collected after centrifugation (15,000 rpm, 4 degrees C., 20 min). Concentrations of amyloid-beta 1-40 peptide were determined by commercially available ECL kit (Meso Scale Discovery).

The concentrations of amyloid-beta 1-40 peptide were shown in FIG. 20. As a result, the concentrations of amyloid-beta 1-40 in brains of IL6R/KLH-TfR-NEP or IL6R/MOG303-TfR-NEP administrated mice are significantly lower at day 1 compared to that of KLH and IL6R/KLH-NEP administrated mice. On the other hand, at day 3 after antibody administration, amyloid-beta 1-40 concentration in brain of IL6R/MOG303-TfR-NEP administrated mouse is the lowest, and is significantly lower compared to that of KLH, IL6R/KLH-NEP or IL6R/KLH-TfR-NEP administrated mouse. The result suggests the longer-lasting of NEP-conjugated IgG in brain by retention moiety (anti-MOG antibody) can make a continuous pharmacodynamic effect in brain compared to normal IgG and IgG which have the brain transfer moiety (anti-TfR antibody) alone.

### Example 12C: Pharmacokinetic Profiles of GAA-Conjugated Antibody in Plasma and Brain in Mice

Pharmacokinetics of the following GAA-conjugated antibodies in plasma and brain were evaluated on C57BL/6J mice (male, 7-8 weeks) to reveal their elimination from plasma, and transferability into and retention of them in brain.

The negative control antibodies (KLH, IL6R/KLH-GAA, KLH-2GAA), the antibodies with the transfer moiety (IL6R/KLH-TfR-GAA, KLH-TfR-2GAA), and the antibodies with brain transfer and retention moieties (IL6R/MOG303-TfR-GAA, MOG303-TfR-2GAA) described in Example 9A were administered intravenously in mice at a dose of 20 nmol/kg. Blood was collected on 1 day and 7 days after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. A half of the brain was dissolved by Cell extraction buffer (Invitrogen) containing Complete Mini Protease Inhibitor Cocktail (Roche) in M tube (Miltenyi) and was homogenized with gentleMACS (Miltenyi). Homogenate was centrifuged (15,000 rpm, 4 degrees C., 10 min) and the supernatant was collected.

Concentrations of the antibodies in plasma and in brain were determined by electro chemiluminescence-immunoassay (ECL). As a capture antibody, an anti-human IgG Fab′2 antibody (LifeSpan BioSciences) was applied in the plate. After PBS with Tween 20 (PBST, Sigma-Aldrich) containing 1% bovine serum albumin (Sigma-Aldrich) was added as blocking solution, diluted plasma or brain homogenate samples were applied. As a detection antibody, a biotin-labeled anti-human IgG antibody (Bethyl Laboratories) was added. Finally, a streptavidin labeled with SULFO-tag (Meso Scale Discovery) was added, and signals were detected by ECL (Meso Scale Discovery).

The negative control antibodies (KLH, IL6R/KLH-shortGAA, KLH-2shortGAA), the antibodies with the transfer moiety (IL6R/KLH-TfR-shortGAA, KLH-TfR-2shortGAA), and the antibodies with brain transfer and retention moieties (IL6R/MOG303-TfR-shortGAA, MOG303-TfR-2shortGAA) described in Example 9A are administered intravenously in mice at a dose of 20 nmol/kg. Blood is collected on 1 day and 7 days after dosing, then plasma is obtained by centrifugation (12,000 rpm, 4 degrees C., 5 min). After blood sampling, phosphate-buffered saline (PBS) is perfused from the heart by inserting a catheter to remove blood, and then brain is collected. A half of the brain is dissolved by Cell extraction buffer (Invitrogen) containing Complete Mini Protease Inhibitor Cocktail (Roche) in M tube (Miltenyi) and is homogenized with gentleMACS (Miltenyi). Homogenate is centrifuged (15,000 rpm, 4 degrees C., 10 min) and the supernatant is collected.

Concentrations of the antibodies in plasma and in brain are determined by electro chemiluminescence-immunoassay (ECL). As a capture antibody, an anti-human IgG Fab′2 antibody (LifeSpan BioSciences) is applied in the plate. After PBS with Tween 20 (PBST, Sigma-Aldrich) containing 1% bovine serum albumin (Sigma-Aldrich) is added as blocking solution, diluted plasma or brain homogenate samples are applied. As a detection antibody, a biotin-labeled anti-human IgG antibody (Bethyl Laboratories) is added. Finally, a streptavidin labeled with SULFO-tag (Meso Scale Discovery) is added, and signals are detected by ECL (Meso Scale Discovery).

### Example 12D: Pharmacokinetic Profiles of Anti-BACE1 Antibody with Brain Transfer and Retention Moieties in Plasma and in Brain

Pharmacokinetics of IgG antibodies of anti-KLH-IgG (KLH), anti-BACE1/KLH-IgG (BACE1/KLH-TfR) and anti-BACE1/MOG303-IgG conjugated with anti-TfR domain (BACE1/MOG303-TfR) described in Example 9C were evaluated on C57BL/6J mice (male, 7-9 weeks) to reveal their elimination from plasma, transferability into, and retention of those antibodies in brain.

KLH, BACE1/KLH-TfR and BACE1/MOG303-TfR were administered intravenously in mice at a dose of 25 mg/kg. Blood was collected at day 2, 7 and 14 after dosing, then plasma was obtained by centrifugation (12,000 rpm, 4 degrees C., 5 min). After blood sampling, phosphate-buffered saline (PBS) was perfused from the heart by inserting a catheter to remove blood, and then brain was collected. A piece of mouse whole brain was dissolved by cell extraction buffer (Invitrogen) containing Complete Mini Protease Inhibitor Cocktail (Roche) in M tube (Miltenyi Biotec) and homogenized with gentleMACS Dissociator (Miltenyi Biotec). Homogenates were centrifuged (15,000 rpm, 4 degrees C., 20 min) and the supernatants were collected.

Concentrations of the antibodies in plasma and in brain were determined by the electro chemiluminescence-immunoassay (ECL) and enzyme-linked immuno sorbent assay (ELISA), respectively. As a capture antibody, an anti-human IgG Fab′2 antibody (LifeSpan BioSciences) was applied in the plate. After PBS with Tween20 (PBST, Sigma-Aldrich) containing 1% bovine serum albumin (Sigma-Aldrich) was added as blocking solution, diluted plasma and brain homogenate samples were applied. As a detection antibody, a biotin-labeled anti-human IgG antibody (Bethyl Laboratories) was added. For ECL, a streptavidin labeled with SULFO-tag (Meso Scale Discovery) was added, and signals were detected by ECL (Meso Scale Discovery). For ELISA, a streptavidin labeled with poly horseradish peroxidase (HRP, Stereospecific Detection Technologies) was added, and signals were detected in plate reader (BMG LABTECH) followed by addition of s 3,3′,5,5′-tetramethylbenzidine as a substrate for HRP (TMB, SURMODICS) and sulfuric acid as a reaction stop solution for HRP.

The concentrations of the antibodies in plasma were shown in FIG. 21(a). BACE1/KLH-TfR and BACE1/MOG303-TfR showed lower concentrations at day 2, 7 and 14, and were eliminated from the plasma much faster than KLH. The result suggests that antibodies with anti-TfR domain were eliminated rapidly from plasma due to the systemic expression of TfR.

The concentrations of the antibodies in brain were shown in FIG. 21(b). BACE1/KLH-TfR and BACE1/MOG303-TfR showed higher concentrations at day 2 and 7 compared with KLH. In addition, BACE1/MOG303-TfR showed higher concentration in brain compared with BACE1/KLH-TfR at day 2, 7 and 14. These results indicate that combination of the brain transfer moiety (anti-TfR antibody) and the brain retention moiety (anti-MOG antibody) have synergistic effect on the potency of the antibody transfer into and retention in brain for such anti-BACE1 antibodies.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Sequence Listing

1. An antigen-binding molecule comprising a first antigen-binding domain and a second antigen-binding domain,
wherein the first antigen-binding domain specifically binds a first target that facilitates transfer of the antigen-binding molecule into a mammalian brain, and
the second antigen-binding domain specifically binds a second target wherein the second target (i) is expressed on the cell membrane of brain cells, or (ii) is a brain extracellular matrix (ECM) protein or a brain ECM polysaccharide.

2. The antigen-binding molecule of claim 1, wherein the first target is a molecule that is expressed on vascular endothelial cells of the blood-brain barrier (BBB).

3. The antigen-binding molecule of claim 1 or 2, wherein the first target is selected from the group consisting of Transferrin receptor (TfR), Basigin (CD147), Glut1, Ldlrad3, CD320, Insulin receptor, insulin-like growth factor 1 receptor (IGF1R), Low density lipoprotein Receptor (LDLR), Low density lipoprotein receptor related protein (LRP), preferably LRP1, Diphtheria toxin Receptor, Glucose receptor, CD98hc, TMEM30A, Leptin receptor (LepR) and heparan sulfate chains branching from proteoglycan (HSPG).

4. The antigen-binding molecule of any one of claims 1 to 3, wherein the second target is selected from the group consisting of Myelin Oligodendrocyte glycoprotein (MOG), Neuroglycan C/Chondroitin sulfate proteoglycan 5 (CSPG5), IGSF4B/SynCAM3 (CADM3), CNPase (2′,3′-cyclic nucleotide 3′-phosphodiesterase), Myelin-associated Glycoprotein (MAG), Myelin Basic Protein (MBP), EAAT1 (Solute Carrier Family 1 member 3), EAAT2 (Solute Carrier Family 1 member 2), MAP2 (Microtubule-associated protein2), NEFL (Neurofilament light polypeptide), NEFM (Neurofilament medium polypeptide), NSE (Gamma-enolase), CD68 (Macrosialin), Allograft inflammatory factor 1 (IBA1 or AIF1), Purinergic receptor (P2RY12), Interleukin 1 receptor accessory protein like 1 (ILIRAPL1), Glutamate ionotropic receptor NMDA type subunit 2B (GRIN2B), Calcium voltage-gated channel auxiliary subunit gamma 8 (CACNG8), CD11b (Integrin subunit alpha M), SLC6A2 (Sodium-dependent noradrenaline transporter), DPP6 (Dipeptidyl peptidase like 6), SLC18A3 (Vesicular acetylcholine transporter), Sodium/potassium-transporting ATPase subunit alpha-2, Broad substrate specificity ATP-binding cassette transporter ABCG2, Solute carrier family 12 member 9, Electrogenic sodium bicarbonate cotransporter 1, Excitatory amino acid transporter 2, Chondroitin sulfate proteoglycan 4, Immunoglobulin superfamily DCC subclass member 4, Vang-like protein 2, Neural cell adhesion molecule 1 (N-CAM-1), Low-density lipoprotein receptor-related protein 4 (LRP-4), Phosphoprotein associated with glycosphingolipid-enriched microdomains 1 (Csk-binding protein), Plasma membrane calcium-transporting ATPase 1, Prominin-1, Somatostatin receptor type 1, Carnitine O-palmitoyltransferase 1 brain isoform (CPT1-B), Epidermal growth factor receptor, Protein MAL2, Syntaxin-1A, Sodium/calcium exchanger 1, Lysophosphatidylcholine acyltransferase 1 (LPC acyltransferase 1), Calsyntenin-3 (Alcadein-beta), Pituitary adenylate cyclase-activating polypeptide type I receptor (PACAP type I receptor), Neutral cholesterol ester hydrolase 1 (NCEH), CD166 antigen (Activated leukocyte cell adhesion molecule), Inactive tyrosine-protein kinase 7, Claudin-11, Ectonucleotide phosphatase (ENPP6), Tetraspanin-2 (Tspan-2), Myelin proteolipid protein (PLP), Glycolipid transfer protein (GLTP), Versican core protein (Chondroitin sulfate proteoglycan 2 or CSPG2), Tropoelastin (Elastin), Collagen alpha-2(IV) chain (Canstatin), Proteoglycan link protein 1 (Hyaluronan and proteoglycan link protein 1), Tenascin-R (TN-R), Proteoglycan link protein 2 (Hyaluronan and proteoglycan link protein 2), Collagen alpha-1(I) chain, Neurofilament-3 (NEF3), Immunoglobulin superfamily member 8 (IgSF8), Laminin subunit gamma-1 (LAMC1), Collagen alpha-1(VI) chain (Col6a1), and Collagen alpha-3(VI) chain (Col6a3).

5. The antigen-binding molecule of any one of claims 1 to 4, wherein the brain cells comprise one or more of the cells selected from the group consisting of oligodendrocytes, astrocytes, neurons and microglia.

6. The antigen-binding molecule of any one of claims 1 to 5, wherein the second target is a molecule that is predominantly expressed on brain-specific cell(s).

7. The antigen-binding molecule of any one of claims 1 to 6, wherein the first target is selected from the group consisting of Transferrin receptor (TfR), Basigin (CD147), Insulin receptor, insulin-like growth factor 1 receptor (IGF1R), Low density lipoprotein Receptor (LDLR), Low density lipoprotein receptor related protein (LRP), preferably LRP1, Diphtheria toxin Receptor, Glucose receptor, preferably Glut1, and CD98hc.

8. The antigen-binding molecule of any one of claims 1 to 7, wherein the second target is selected from the group consisting of Myelin Oligodendrocyte glycoprotein (MOG), Neuroglycan C/Chondroitin sulfate proteoglycan 5 (CSPG5), and Cell Adhesion Molecule 3 (CADM3).

9. The antigen-binding molecule of any one of claims 1 to 8, wherein:
(i) the first target is Transferrin receptor (TfR) and the second target is Myelin Oligodendrocyte glycoprotein (MOG); or
(ii) the first target is Transferrin receptor (TfR) and the second target is Neuroglycan C/Chondroitin sulfate proteoglycan 5 (CSPG5); or
(iii) the first target is Transferrin receptor (TfR) and the second target is Cell Adhesion Molecule 3 (CADM3); or
(iv) the first target is Basigin (CD147) and the second target is Myelin Oligodendrocyte glycoprotein (MOG); or
(v) the first target is Basigin (CD147) and the second target is Neuroglycan C/Chondroitin sulfate proteoglycan 5 (CSPG5); or
(vi) the first target is Basigin (CD147) and the second target is Cell Adhesion Molecule 3 (CADM3); or
(vii) the first target is Insulin receptor and the second target is Myelin Oligodendrocyte glycoprotein (MOG); or
(viii) the first target is Insulin receptor and the second target is Neuroglycan C/Chondroitin sulfate proteoglycan 5 (CSPG5); or
(ix) the first target is Insulin receptor and the second target is Cell Adhesion Molecule 3 (CADM3); or
(x) the first target is glucose receptor and the second target is Myelin Oligodendrocyte glycoprotein (MOG); or
(xi the first target is glucose receptor and the second target is Neuroglycan C/Chondroitin sulfate proteoglycan 5 (CSPG5); or
(xii) the first target is glucose receptor and the second target is Cell Adhesion Molecule 3 (CADM3); or
(xiii) the first target is Low density lipoprotein Receptor (LDLR) and the second target is Myelin Oligodendrocyte glycoprotein (MOG); or
(xiv) the first target is Low density lipoprotein Receptor (LDLR) and the second target is Neuroglycan C/Chondroitin sulfate proteoglycan 5 (CSPG5); or
(xv) the first target is Low density lipoprotein Receptor (LDLR) and the second target is Cell Adhesion Molecule 3 (CADM3); or
(xvi) the first target is Low density lipoprotein receptor related protein (LRP) and the second target is Myelin Oligodendrocyte glycoprotein (MOG); or
(xvii) the first target is Low density lipoprotein receptor related protein (LRP) and the second target is Neuroglycan C/Chondroitin sulfate proteoglycan 5 (CSPG5); or
(xviii) the first target is Low density lipoprotein receptor related protein (LRP) and the second target is Cell Adhesion Molecule 3 (CADM3); or
(xix) the first target is CD98hc and the second target is Myelin Oligodendrocyte glycoprotein (MOG); or
(xx) the first target is CD98hc and the second target is Neuroglycan C/Chondroitin sulfate proteoglycan 5 (CSPG5); or
(xxi) the first target is CD98hc and the second target is Cell Adhesion Molecule 3 (CADM3).

10. The antigen-binding molecule of any one of claims 1 to 4, wherein the second target is not a target selected from the group consisting of beta-secretase 1 (BACE1), Abeta, epidermal growth factor receptor (EGFR), human epidermal growth factor receptor 2 (HER2), tau, apolipoprotein E (ApoE), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, TRK A, TRK B, TRK C, an alpha synuclein, a beta synuclein, a gamma synuclein, vascular endothelial growth factor (VEGF), neuropilin, a Semaphorin, Semaphorin 3A, Semaphorin 4A, Semaphorin 6A, myelin basic protein (MBP), MOG, PLP, MAG, aquaporin 4, glutamate receptor, and EpCAM.

11. The antigen-binding molecule of any one of claims 1 to 10, further comprising
(i) at least one functional moiety comprising an enzyme, a therapeutic protein, an antibody or antigen-binding fragment thereof, a peptide, a DNA, an shRNA, an siRNA, a small molecule drug, or a cytotoxic agent, and/or
(ii) at least one in vivo half-life extension moiety, preferably wherein said at least one in vivo half-life extension moiety is selected from the group consisting of an Fc region, an albumin-binding domain, an FcRn-binding protein, an FcRn-binding peptide and a PEG moiety.

12. A method for increasing the concentration of an antigen-binding molecule in the brain of a subject in need thereof, the method comprising:
(a) providing a first antigen-binding molecule comprising:
(a1) a first antigen-binding domain that specifically binds a first target that facilitates transfer of the antigen-binding molecule into a mammalian brain; or
(a2) a second antigen-binding domain that specifically binds a second target wherein the second target (i) is expressed on the cell membrane of brain cells, or (ii) is a brain ECM protein or a brain ECM polysaccharide;
(b) introducing
in the case of (a1), to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule; or
in the case of (a2), to the first antigen-binding molecule at least one first antigen-binding domain that specifically binds a first target that facilitates transfer of the antigen-binding molecule into a mammalian brain, thereby obtaining a second antigen-binding molecule;
such that the concentration of said second antigen-binding molecule in the brain of said subject is increased upon administration to said subject as compared to the first antigen-binding molecule.

13. The method of claim 12, further comprising step (c):
(c) determining that the concentration of said second antigen-binding molecule in the brain of said subject is increased compared to a control antigen-binding molecule, wherein the control antigen-binding molecule differs from the second antigen-binding molecule according to (b) only in that it does not comprise:
in the case of (a1), said at least one second antigen-binding domain that specifically binds the second target wherein the second target (i) is expressed on the cell membrane of brain cells, or (ii) is a brain ECM protein or a brain ECM polysaccharide; or
in the case of (a2) said at least one first antigen-binding domain that specifically binds the first target that facilitates transfer of the antigen-binding molecule into a mammalian brain.

14. The method of claim 12 or 13, wherein the concentration of said second antigen-binding molecule in the brain of said subject is Cmax.

15. A method for increasing exposure of an antigen-binding molecule in the brain of a subject in need thereof, the method comprising:
(a) providing a first antigen-binding molecule comprising:
(a1) a first antigen-binding domain that specifically binds a first target that facilitates transfer of the antigen-binding molecule into a mammalian brain; or
(a2) a second antigen-binding domain that specifically binds a second target wherein the second target (i) is expressed on the cell membrane of brain cells, or (ii) is a brain ECM protein or a brain ECM polysaccharide;
(b) introducing
in the case of (a1), to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule; or
in the case of (a2), to the first antigen-binding molecule at least one first antigen-binding domain that specifically binds a first target that facilitates transfer of the antigen-binding molecule into a mammalian brain, thereby obtaining a second antigen-binding molecule;
such that the exposure of said second antigen-binding molecule in the brain of said subject is increased upon administration to said subject as compared to the first antigen-binding molecule.

16. The method of claim 15, further comprising step (c):
(c) determining that the exposure of said second antigen-binding molecule in the brain of said subject is increased compared to a control antigen-binding molecule, wherein the control antigen-binding molecule differs from the second antigen-binding molecule according to (b) only in that it does not comprise
in the case of (a1), said at least one second antigen-binding domain that specifically binds the second target wherein the second target (i) is expressed on the cell membrane of brain cells, or (ii) is a brain ECM protein or a brain ECM polysaccharide; or
in the case of (a2) said at least one first antigen-binding domain that specifically binds the first target that facilitates transfer of the antigen-binding molecule into a mammalian brain.

17. The method of claim 15 or 16, wherein the exposure of said second antigen-binding molecule in the brain of said subject is the AUC (Area Under Curve) of brain concentration-time profiles of the antigen-binding molecule.

18. A method for the retention of an antigen-binding molecule in the brain of a subject in need thereof, the method comprising:
(a) providing a first antigen-binding molecule comprising a first antigen-binding domain that specifically binds a first target that facilitates transfer of the antigen-binding molecule into a mammalian brain;
(b) introducing to the first antigen-binding molecule at least one second antigen-binding domain that specifically binds a second target wherein the second target (i) is expressed on the cell membrane of brain cells, or (ii) is a brain ECM protein or a brain ECM polysaccharide, thereby obtaining a second antigen-binding molecule,
such that the retention of said second antigen-binding molecule in the brain of said subject is increased upon administration to said subject as compared to the first antigen-binding molecule.

19. The method of claim 18, further comprising step (c):
(c) determining that the retention of said second antigen-binding molecule in the brain of said subject is increased compared to a control antigen-binding molecule, wherein the control antigen-binding molecule differs from the second antigen-binding molecule according to (b) only in that it does not comprise said at least one antigen-binding domain that specifically binds the second target.

20. The method of claim 18 or 19, wherein the retention of said second antigen-binding molecule in the brain of said subject is the half-life in vivo in the brain.

21. The antigen-binding molecule of any one of claims 1 to 11 or the method of any one of claims 12 to 20, wherein the mammalian brain is human brain.
